(19) United States
(12) Patent Application Publication
(10) Pub. No.: US 2024/0034775 A1
(43) Pub. Date: Feb. 1, 2024

(54) COMPOSITIONS AND METHODS FOR TREATING EPILEPSY

(71) Applicants: The J. David Gladstone Institutes, a testamentary trust established under the Will of J. David Glads, San Francisco, CA (US); Annexon, Inc., Brisbane, CA (US)

(72) Inventors: Jeanne T. Paz, San Francisco, CA (US); Sethu Sankaranarayanan, Fremont, CA (US); Ted Yednock, Forest Knolls, CA (US)

(21) Appl. No.: 17/923,456

(22) PCT Filed: May 5, 2021
(86) PCT No.: PCT/US21/30930

§ 371 Date: Nov. 7, 2022

Related U.S. Application Data
(60) Provisional application No. 63/020,245, filed on May 5, 2020.

(51) Int. Cl. C07K 16/18; G01N 33/68; A61P 25/08

(52) U.S. Cl. CPC C07K 16/18; G01N 33/6896; A61P 25/08; G01N 2800/2857; G01N 2333/4716; A61K 2039/505

(57) ABSTRACT

The present disclosure relates generally to methods of preventing, reducing risk of developing, or treating epilepsy, comprising administering to a subject an inhibitor of the classical complement pathway.

### RELATED APPLICATIONS

This patent application claims priority to U.S. Provisional Patent Application No. 63/020,245, filed May 5, 2020, which is hereby incorporated by reference in its entirety.

### STATEMENT REGARDING FEDERALLY SPONSORED RESEARCH

This invention was made with government support under W81XWH-16-1-0576 awarded by ARMY/MRMC. The government has certain rights in the invention.

### SEQUENCE LISTING

The instant application contains a Sequence Listing which has been submitted electronically in ASCII format and is hereby incorporated by reference in its entirety. Said ASCII copy, created on May 3, 2021, is named ANH-01025_SL.txt and is 41,770 bytes in size.

### BACKGROUND

Epilepsy is a common neurological disorder and has been identified as one of the most prevalent neurological disorders with neural cell damage or loss. According to estimates from the World Health Organization, approximately 50 million people are affected by epilepsy worldwide and close to 80% of affected individuals reside in developing nations. One of every ten people will have at least one epileptic seizure during a normal lifespan, and a third of these will develop epilepsy. While all age groups can be affected by epileptic seizures, the disorder is most prevalent among the young and elderly. Epilepsy is one of the most common serious neurological disorders in the United States and often requires long-term management. Each year, 150,000 people in the United States are newly diagnosed as having epilepsy.

Despite the availability of recent antiepileptic drugs (ezogabine, pregabalin, levetiracetam, lamotrigine, topiramate, valproate, rufinamide, gabapentin, carbamazepine, clonazepam, oxcarbazepine, phenobarbital and phenytoin), available treatment options are not efficacious enough to prevent or treat the disease and seizures remain difficult to eradicate completely; approximately one-third of patients still have uncontrolled seizures and an even larger percentage suffer from at least one anticonvulsant-related side-effect (e.g., mood changes, sleepiness, or unsteadiness in gait). Furthermore, although seizures represent the most dramatic hallmark of epilepsy, many epilepsy patients develop neurological or psychiatric disease (memory or cognitive impairment, depression . . . ). For example, mesial temporal lobe epilepsy is usually accompanied by memory deficits probably due to hippocampal system damages and/or brain inflammation.

Although the treatment for epilepsy has evolved in the last decade, available treatment options are focused on preventing seizures once they are underway, and the current medications may not cure or even improve the course of disease. Currently available antiepileptic drugs do not seem to be antiepileptogenic. This could be due to the fact that the current agents act in mechanistically inappropriate ways to prevent disease progression. Therefore, there is a need in the art for new therapies to prevent and treat epilepsy.

### SUMMARY

The present disclosure is generally directed to methods of preventing, reducing risk of developing, or treating epilepsy by inhibiting complement activation, e.g., by inhibiting the classical complement pathway.

The present disclosure is generally directed to methods of preventing, reducing risk of developing, or treating epilepsy, such as an idiopathic generalized epilepsy, idiopathic partial epilepsy, symptomatic generalized epilepsy or symptomatic partial epilepsy by inhibiting classical complement activation, e.g., by inhibiting complement factor C1q, C1r, or C1s, e.g., through the administration of antibodies, such as monoclonal, chimeric, humanized antibodies, antibody fragments, antibody derivatives, etc., which bind to one or more of these complement factors.

In some embodiments, the activity of complement factors such as C1q, C1r, or C1s are inhibited to block activation of the classical complement pathway, and slow or prevent epilepsy, such as an idiopathic generalized epilepsy, idiopathic partial epilepsy, symptomatic generalized epilepsy or symptomatic partial epilepsy. Inhibition of the classical complement pathway leaves the lectin and alternative complement pathways intact to perform their normal immune function. Methods related to neutralizing complement factors such as C1q, C1r, or C1s in epilepsy, such as an idiopathic generalized epilepsy, idiopathic partial epilepsy, symptomatic generalized epilepsy or symptomatic partial epilepsy are disclosed herein.

In one aspect, the disclosure provides a method of preventing, reducing risk of developing, or treating epilepsy, such as an idiopathic generalized epilepsy, idiopathic partial epilepsy, symptomatic generalized epilepsy or symptomatic partial epilepsy (e.g., temporal lobe epilepsy), comprising administering to a subject an inhibitor of the classical complement pathway is provided.

Numerous embodiments are further provided that can be applied to any aspect of the present invention described herein. For example, in some embodiments, the inhibitor is administered during or within the first 4 weeks after a seizure, during or within the first week after a seizure, during or within 24 hours after a seizure, or during or within 1, 2, 3, 4, 5, or 6 hours after a seizure. In some embodiments, the inhibitor inhibits synapse loss induced by the seizure. In some embodiments, the inhibitor is administered to a patient suffering from a traumatic brain injury, hypoxic brain injury, brain infection, stroke, or genetic syndrome. The brain infection may be encephalitis, meningitis, mesial temporal sclerosis, or a cerebral tumor. The epilepsy may be induced by the traumatic brain injury, hypoxic brain injury, brain infection, stroke, or genetic syndrome. Epilepsy may be a TBI-induced epilepsy. In some embodiments, the inhibitor inhibits synapse loss induced by the traumatic brain injury, hypoxic brain injury, brain infection, stroke, or genetic syndrome. In some embodiments, the inhibitor is administered during or within the first 4 weeks after a traumatic brain injury, hypoxic brain injury, brain infection, or stroke, during or within the first week after a traumatic brain injury, hypoxic brain injury, brain infection, or stroke, during or within 24 hours after a traumatic brain injury, hypoxic brain injury, brain infection, or stroke, or during or within 1, 2, 3, 4, 5, or 6 hours after a traumatic brain injury, hypoxic brain injury, brain infection, or stroke.

In some embodiments, the inhibitor of the classical complement pathway is a C1q inhibitor. For example, the C1q inhibitor may be an antibody, an aptamer, an antisense nucleic acid or a gene editing agent. In some embodiments, the antibody is an anti-C1q antibody. In some embodiments, the anti-C1q antibody inhibits the interaction between C1q and an autoantibody or between C1q and C1r, or between C1q and C1s. In some embodiments, the anti-C1q antibody promotes clearance of C1q from circulation or a tissue. In some embodiments, the antibody is an anti-C1q antibody having a dissociation constant (KD) that ranges from 100 nM to 0.005 nM or less than 0.005 nM. In some embodiments, the antibody is an anti-C1q antibody that binds C1q with a binding stoichiometry that ranges from 20:1 to 1.0:1 or less than 1.0:1, binds C1q with a binding stoichiometry that ranges from 6:1 to 1.0:1 or less than 1.0:1, or binds C1q with a binding stoichiometry that ranges from 2.5:1 to 1.0:1 or less than 1.0:1. In some embodiments, the antibody specifically binds to and neutralizes a biological activity of C1q, such as (1) C1q binding to an autoantibody, (2) C1q binding to C1r, (3) C1q binding to C1s, (4) C1q binding to IgM, (5) C1q binding to IgG, (6) C1q binding to phosphatidylserine, (7) C1q binding to pentraxin-3, (8) C1q binding to C-reactive protein (CRP), (9) C1q binding to globular C1q receptor (gC1qR), (10) C1q binding to complement receptor 1 (CR1), (11) C1q binding to beta-amyloid, (12) C1q binding to calreticulin, (13) C1q binding to apoptotic cells, or (14) C1q binding to B cells. Another example of the biological activity is (1) activation of the classical complement activation pathway, (2) activation of antibody and complement dependent cytotoxicity, (3) CH50 hemolysis, (4) synapse loss, (5) B-cell antibody production, (6) dendritic cell maturation, (7) T-cell proliferation, (8) cytokine production (9) microglia activation, (10) immune complex formation, (11) phagocytosis of synapses or nerve endings, (12) activation of complement receptor 3 (CR3/C3) expressing cells or (13) neuroinflammation. In some embodiments, CH50 hemolysis comprises human, mouse, rat, dog, rhesus, and/or cynomolgus monkey CH50 hemolysis. In some embodiments, the antibody is capable of neutralizing from at least about 50%, to at least about 90% of CH50 hemolysis, or neutralizing at least 50% of CH50 hemolysis at a dose of less than 150 ng/ml, less than 100 ng/ml, less than 50 ng/ml, or less than 20 ng/ml.

The antibody may be a monoclonal antibody, a polyclonal antibody, a recombinant antibody, a humanized antibody, a chimeric antibody, a multispecific antibody, antibody fragments, or an antibody derivative thereof, such as a Fab fragment, a Fab′ fragment, a F(ab′)2 fragment, a Fv fragment, a diabody, or a single chain antibody molecule. The antibody may be coupled to a labeling group, such as an optical label, radioisotope, radionuclide, an enzymatic group, biotinyl group, a nucleic acid, oligonucleotide, enzyme, or a fluorescent label.

In certain preferred embodiments, the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7. Similarly, in certain preferred embodiments, the antibody comprises a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7. In some embodiments, the light chain variable domain comprising an amino acid sequence selected from SEQ ID NO: 4 and 35-38. In some embodiments, the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the heavy chain variable domain comprising an amino acid sequence selected from SEQ ID NO: 8 and 31-34. In other preferred embodiments, the antibody fragment comprises heavy chain Fab fragment of SEQ ID NO: 39 and light chain Fab fragment of SEQ ID NO: 40.

In other embodiments, the inhibitor of the classical complement pathway is a C1r inhibitor. In some embodiments, the C1r inhibitor is an antibody, an aptamer, an antisense nucleic acid or a gene editing agent. In some embodiments, the antibody is an anti-C1r antibody. In some embodiments, the anti-C1r antibody inhibits the interaction between C1r and C1q or between C1r and C1s, or wherein the anti-C1r antibody inhibits the catalytic activity of C1r or inhibits the processing of pro-C1r to an active protease. In some embodiments, the antibody is an anti-C1r antibody having a dissociation constant (KD) that ranges from 100 nM to 0.005 nM or less than 0.005 nM. In some embodiments, the antibody is an anti-C1r antibody that binds C1r with a binding stoichiometry that ranges from 20:1 to 1.0:1 or less than 1.0:1, ranges from 6:1 to 1.0:1 or less than 1.0:1, or ranges from 2.5:1 to 1.0:1 or less than 1.0:1. In some embodiments, the anti-C1r antibody promotes clearance of C1r from circulation or a tissue.

In other embodiments, the inhibitor of the classical complement pathway is a C1s inhibitor. The C1s inhibitor may be an antibody, an aptamer, an antisense nucleic acid or a gene editing agent. In some embodiments, the antibody is an anti-C1s antibody. In some embodiments, the anti-C1s antibody inhibits the interaction between C1s and C1q or between C1s and C1r or between C1s and C2 or C4, or wherein the anti-C1s antibody inhibits the catalytic activity of C1s or inhibits the processing of pro-C1s to an active protease or binds to an activated form of C1s. In some embodiments, the antibody is an anti-C1s antibody having a dissociation constant (KD) that ranges from 100 nM to 0.005 nM or less than 0.005 nM. In some embodiments, the antibody is an anti-C1s antibody that binds C1s with a binding stoichiometry that ranges from 20:1 to 1.0:1 or less than 1.0:1, ranges from 6:1 to 1.0:1 or less than 1.0:1, or ranges from 2.5:1 to 1.0:1 or less than 1.0:1. In some embodiments, the anti-C1s antibody promotes clearance of C1s from circulation or a tissue.

In other embodiments, the inhibitor of the classical complement pathway is an anti-C1 complex antibody, optionally wherein the anti-C1 complex antibody inhibits C1r or C1s activation or prevents their ability to act on C2 or C4, e.g., the anti-C1 complex antibody binds to a combinatorial epitope within the C1 complex, wherein said combinatorial epitope comprises amino acids of both C1q and C1s; both C1q and C1r; both C1r and C1s; or each of C1q, C1r, and C1s. The antibody may be a monoclonal antibody. In some embodiments, the antibody inhibits cleavage of C4 and does not inhibit cleavage of C2, or inhibits cleavage of C2 and does not inhibit cleavage of C4.

In some embodiments, the antibody binds mammalian C1q, C1r, or C1s, or binds human C1q, C1r, or C1s. In some embodiments, the antibody binds mammalian C1 complex.

In some embodiments, the antibody is a mouse antibody, a human antibody, a humanized antibody, or a chimeric antibody. In some embodiments, the antibody is an antibody fragment selected from Fab, Fab′-SH, Fv, scFv, and F(ab′)2 fragments. In some embodiments, the antibody is a bispecific antibody recognizing a first antigen and a second antigen. For example, the first antigen may be selected from C1q, C1r, and C1s and the second antigen may be an antigen that facilitates transport across the blood-brain-barrier. The second antigen may be transferrin receptor (TR), insulin receptor (HIR), insulin growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, or ANG1005.

In some embodiments, the antibody inhibits the classical complement activation pathway by an amount that ranges from at least 30% to at least 99.9%. In some embodiments, the antibody inhibits the alternative complement activation pathway initiated by C1q binding.

In some embodiments, the antibody inhibits the alternative complement activation pathway by an amount that ranges from at least 30% to at least 99.9%. In some embodiments, the antibody inhibits complement-dependent cell-mediated cytotoxicity (CDCC), e.g., the antibody inhibits complement-dependent cell mediated cytotoxicity (CDCC) activation pathway by an amount that ranges from at least 30% to at least 99.9%. In some embodiments, the antibody inhibits autoantibody and complement-dependent cell-mediated cytotoxicity (CDCC).

In some embodiments, the method further comprises administering a second antibody selected from an anti-C1q antibody, an anti-C1r antibody, and an anti-C1s antibody. In some embodiments, the method further comprises administering to the subject a therapeutically effective amount of an inhibitor of antibody-dependent cellular cytotoxicity (ADCC). In some embodiments, the method further comprises administering to the subject a therapeutically effective amount of an inhibitor of the classical complement activation pathway. In some embodiments, the method further comprises administering to the subject a therapeutically effective amount of an inhibitor of the alternative complement activation pathway. In some embodiments, the method further comprises administering to the subject a therapeutically effective amount of an inhibitor of an interaction between the autoantibody and its correspond autoantigen.

In another aspect, a method of determining a subject's risk of developing epilepsy due to a traumatic brain injury, hypoxic brain injury, brain infection, stroke, or genetic syndrome, comprising: (a) administering an anti-C1q, anti-C1r, or anti-C1s antibody to the subject, wherein the anti-C1q, anti-C1r, or anti-C1s antibody is coupled to a detectable label; (b) detecting the detectable label to measure the amount or location of C1q, C1r, or C1s in the subject; and (c) comparing the amount or location of one or more of C1q, C1r, or C1s to a reference, wherein the risk of developing epilepsy is characterized based on the comparison of the amount or location of one or more of C1q, C1r, or C1s to the reference, is provided. In some embodiments, the brain infection is encephalitis, meningitis, mesial temporal sclerosis, or a cerebral tumor. The detectable label may comprise a nucleic acid, oligonucleotide, enzyme, radioactive isotope, biotin or a fluorescent label. In some embodiments, the antibody is an antibody fragment selected from Fab, Fab′-SH, Fv, scFv, and F(ab′)2 fragments.

In certain preferred embodiments, the anti-C1q antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7. In some embodiments, the anti-C1q antibody comprises a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the anti-C1q antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7. In some embodiments, the light chain variable domain comprising an amino acid sequence selected from SEQ ID NO: 4 and 35-38. In some embodiments, the anti-C1q antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the heavy chain variable domain comprising an amino acid sequence selected from SEQ ID NO: 8 and 31-34.

In some embodiments, the epilepsy is an idiopathic generalized epilepsy, idiopathic partial epilepsy, symptomatic generalized epilepsy or symptomatic partial epilepsy (e.g., temporal lobe epilepsy). In some embodiments, the symptomatic generalized epilepsy or the symptomatic partial epilepsy is induced by traumatic brain injury.

### DESCRIPTION OF THE FIGURES

FIGS. 1A-1E depicts that the injured cortex and functionally connected thalamus show chronic inflammation and neuron loss three weeks after mTBI. FIGS. 1A-1B show schematic of a mouse coronal brain section showing the site and depth of the controlled cortical impact (FIG. 1A) and the location of the S1 cortex and nRT and VB thalamic regions (FIG. 1B). The impactor has a diameter of 3 mm and the impact was delivered at a depth of 0.8 mm to the right somatosensory cortex. FIG. 1C shows representative coronal brain section from a mTBI mouse stained for C1q. Bilateral C1q expression in the hippocampus is typical of physiological conditions and is present in both sham and mTBI mice. FIG. 1D shows close-up images of S1 (top), VB and nRT (middle), and confocal images of nRT (bottom) stained for C1q, neuronal marker NeuN, astrocyte marker GFAP, and microglia/macrophage marker IBA1. Injury site in the right S1 cortex is marked by an asterisk. Arrow in nRT indicates location of confocal image. Scale bars, 300 μm (top/middle) and 20 μm (bottom). FIG. 1E shows quantification of fluorescence ratios between ipsilateral and contralateral regions in sham and mTBI mice. Data represent all points from min to max, with a Mann-Whitney test and α=0.05 (*p<0.05, **p<0.01). Analysis includes between five and seven mice per group (n=three sections per mouse, one image per region).

FIGS. 2A-2I depict that the nRT ipsilateral to the injured cortex shows neuron loss and altered IPSC and EPSC properties three weeks after mTBI. FIGS. 2A-2C show high-magnification coronal image of the nRT showing divisions into “head”, “body”, and “tail” (FIG. 2A), and quantification of neuron counts across the entire ipsilateral nRT (FIG. 2B) or per subdivision, normalized to the median value from the sham group (FIG. 2C). Neuron count data represent mean±SEM, with a Mann-Whitney test and α=0.05 (*p<0.05, **p<0.01). Analysis includes six mice per group (n=three sections per mouse, averaged). FIGS. 2D-2E show spontaneous IPSC recordings (FIG. 2D) from representative nRT neurons in sham and mTBI mice, and frequency and amplitude distributions (FIG. 2E) in 13 posterior nRT neurons from four sham mice and 22 posterior nRT neurons from six mTBI mice. IPSC data represent mean±SEM analyzed with a Mann-Whitney test and α=0.05 (*p<0.05). FIGS. 2F-2G show spontaneous EPSC recordings (FIG. 2F) from representative nRT neurons in sham and mTBI mice, and frequency and amplitude distributions (FIG. 2G) in 11 posterior nRT neurons from six sham mice and nine posterior nRT neurons from seven mTBI mice. Inset shows averaged EPSC traces from single nRT neurons from sham and mTBI mice, plotted on the same scale. EPSC data represent mean±SEM analyzed with a Mann-Whitney test and α=0.05 (*p<0.05). FIG. 2H shows representative images of coronal brain sections from Thy1-GCaMP6f mice with sham surgery (left) and mTBI (right) (injury site marked by asterisk). Bottom panels show projection terminals from the cortex to VB and nRT. Scale bars, 1 mm (top) and 500 μm (bottom). Reduction in projection terminals from the cortex to VB and nRT (marked by arrows) were observed in n=six mTBI mice. FIG. 2I shows quantification of Thy1-GCaMP fluorescence ratios between ipsilateral and contralateral regions in sham and mTBI mice. Data represent all points from min to max, with a Mann-Whitney test and α=0.05 (*p<0.05, **p<0.01). Analysis includes five sham mice and six mTBI mice (n=three sections per mouse, one image per region).

FIGS. 3A-3H depict that single-nucleus RNA sequencing shows that microglia are the source of C1q in the thalamus three weeks after mTBI. FIG. 3A depicts schematic of coronal brain sections showing the location of thalamic tissue dissection. FIGS. 3B, 3C, and 3E show uniform manifold approximation and projection (UMAP) projection of single nuclei (n=4,908 sham cells, n=4,338 mTBI cells, after data cleaning) colored by cell type lineage (FIG. 3B), nuclear C1qa expression (FIG. 3C), or C4b expression (FIG. 3E). Lineage markers described in FIG. 8A. Coloring is rendered using imputation. Normalized expression scale shown above, 0-max, with max value for each panel. FIGS. 3D and 3F show violin plots of C1qa expression in microglial nuclei (FIG. 3D) and of C4b expression in oligodendrocyte nuclei (FIG. 3F) from cluster 3 (Oligo 3, FIG. 9E) from sham and mTBI mice, analyzed with a Wilcoxon Rank Sum test (n.s.=not significant). Analysis combines both technical replicates, collectively representing nine sham mice and ten mTBI mice. Each dot represents a single nucleus. FIGS. 3G and 3H show RT-qPCR quantification of C1qa (FIG. 3E) and C4b (FIG. 3H) transcripts in bulk cytoplasmic RNA. Each dot represents bulk RNA extracted from one replicate (n=two biological pools, each point represents n=three technical replicates). The first replicate includes five sham mice and six mTBI mice, and the second replicate includes four sham mice and four mTBI mice.

FIGS. 4A-4C show that anti-C1q antibody reduces chronic inflammation and neuron loss three weeks after mTBI. FIGS. 4A and 4B show representative coronal brain sections (FIG. 4A) and close-ups (FIG. 4B) of S1 (top), VB and nRT (bottom) from mTBI mice treated with anti-C1q antibody and stained for C1q, NeuN, GFAP, and IBA1. Injury site in the right S1 cortex is marked by an asterisk. Scale bars, 1 mm (A), 500 μm (B). FIG. 4C shows quantification of nRT neuron counts and fluorescence ratios between ipsilateral and contralateral regions in control and antibody-treated sham and mTBI mice. Data represent all points from min to max, with a Mann-Whitney test and α=0.05 (*p<0.05, **p<0.01). Analysis includes between six and eight mice per group (n=three sections per mouse, one image per region).

FIGS. 5A-5H depict that chronically recorded mTBI mice show altered power across different ECoG frequency bands. FIG. 5A shows representative 10-minute spectrograms from a sham mouse (left) and mTBI mouse (right) taken at the same time point within the first 24 hours of mTBI, overlaid with ECoG traces from ipsilateral S1. The mTBI spectrogram shows an electrographic seizure, while the sham spectrogram shows normal ECoG activity. Color bar represents power (mV2/Hz). FIG. 5B shows representative seven-day spectrograms from a sham mouse (left) and mTBI mouse (right) showing power across different frequency bands two to three weeks post-mTBI. Power bands are sampled every 30 minutes. Color bar represents power (mV2/Hz). FIGS. 5C, 5E, and 5G show power spectral density of ECoG activity from sham and mTBI cohorts averaged across the first (FIG. 5C), third (FIG. 5E) and 11th (FIG. 5G) week post mTBI. Inset in C shows examples of power spectral density plots from a representative sham and mTBI mouse. See methods for details. FIGS. 5D, 5F, and 5H show two-way ANOVAs of average power across frequency bands for the first (FIG. 5D), third (FIG. 5F) and 11th (FIG. 5H) week post mTBI. Each dot represents power for one mouse. Data represent all mice recorded, analyzed with a two-way ANOVA (*p<0.05, **p<0.01), even if they died or if the battery ran out before the experimental endpoint. n=seven sham mice, 11 mTBI mice. One mouse died within two days post-mTBI. The remaining mice were recorded for the first week post-mTBI, then recorded for alternating weeks until eleven weeks post-mTBI. Delta=1-4 Hz, theta=5-8 Hz, alpha=9-12 Hz, sigma=13-15 Hz, beta=16-30 Hz, gamma=31-50 Hz.

FIGS. 6A-6E show that anti-C1q antibody has modest effects on ECoG spectral features in mice with mTBI. FIG. 6A shows example spectrograms (top) and histograms (bottom) from a control-treated mouse (left) and antibody-treated mouse (right) showing power across different frequency bands one month post-mTBI. Power bands are sampled every 30 minutes. Color bar represents power (mV2/Hz). FIGS. 6B and 6D show power spectral density of ECoG activity from control-treated and antibody-treated mTBI cohorts averaged across the first (FIG. 6B) or third (FIG. 6D) week post-mTBI. Inset shows an example of power spectral density plots from a representative control-treated mTBI mouse and an antibody-treated mTBI mouse. FIGS. 6C and 6E show two-way ANOVAs of average power across frequency bands for the first (FIG. 6C) and third (FIG. 6E) week post-mTBI. Each dot represents power for one mouse. Data represent all mice recorded, analyzed with a two-way ANOVA, even if they died before treatment ended. n=seven control-treated mice, seven antibody-treated mice. Delta=1-4 Hz, theta=5-8 Hz, alpha=9-12 Hz, sigma=13-15 Hz, beta=16-30 Hz, gamma=31-50 Hz.

FIGS. 7A-7I show postmortem brain tissue from a patient with TBI shows chronic inflammation eight days after TBI. FIG. 7A shows postmortem brain tissue from one control patient stained for HLA-DR, a marker for an MHC class II cell surface receptor that is expressed in microglia and macrophages. Case information: male, age 78. Scale bar, 1 cm. FIG. 7B shows postmortem brain tissue from one TBI patient stained for HLA-DR. Case information: male, age 79; fall accident, Injury Severity (GCS): moderate, CT: cerebral edema; no epilepsy (post-TBI: eight days); no history of neurological diseases and without evidence of cognitive decline, based on the last clinical evaluation; no evidence of primary neurodegenerative pathology, evidence of trauma-induced diffuse axonal damage. Scale bar, 1 cm. FIG. 7C shows same as (FIG. 7A) but stained for GFAP. Scale bar, 1 cm. FIG. 7D shows same as (FIG. 7B) but stained for GFAP. Scale bar, 1 cm. FIG. 7E shows same as (FIG. 7A) but stained for C1q. Scale bar, 1 cm. FIGS. 7F-7I show same as (FIG. 7B) but stained for C1q. Scale bars, 1 cm (FIGS. 7F-7G) and 40 μm (FIGS. 7H-7I).

FIGS. 8A-8C show that single-nucleus RNA sequencing does not show major differences in expression between sham and mTBI thalamic tissue three weeks after mTBI. FIG. 8A shows violin plots of key lineage genes used to define each cluster in FIG. 3B. FIG. 8B shows UMAP plot of sham and mTBI nuclei, colored by replicate (rep 1: n=five sham mice, n=six mTBI mice, rep 2: n=four sham mice, n=four mTBI mice). FIG. 8C shows percent of nuclei collected for sham (n=4,908) and mTBI (n=4,338) thalamic tissue in each of the lineages as defined in A). Recovery of each lineage is similar between replicates. Each dot represents a biological replicate.

FIGS. 9A-9H show that C1qa and C4b are the main complement markers found in thalamic tissue three weeks after mTBI. FIG. 9A shows expression levels of Apoe and Cst3 in microglia and Apoe and C1u in astrocytes in sham and mTBI, analyzed with a Wilcoxon Rank Sum test (adjusted p-values shown above each violin plot). FIG. 9B shows violin plots for components of the complement system in the main lineages of cells profiled from sham and mTBI thalamic tissue. FIG. 9C shows subclustering of oligodendrocyte lineages. FIG. 9D shows same UMAP as in (FIG. 9C) colored by expression of C4b, rendered with imputation. FIG. 9E shows violin plot of C4b expression in sham and mTBI oligodendrocyte subclusters analyzed with a Wilcoxon Rank Sum test. C4b is differentially expressed in oligodendrocyte cluster 3 (Oligo 3). Analysis combines both technical replicates, collectively representing nine sham mice and ten mTBI mice. Each dot represents a single nucleus. FIG. 9F shows same UMAP as in (FIG. 9C) colored by expression of Kirrel3 and Opalin, associated with mature oligodendrocytes, Enpp6, associated with differentiating oligodendrocytes, and 1133, an alarmin associated with oligodendrocyte maturation. FIG. 9G shows RT-qPCR of C2 expression from RNA extracted from bulk cytoplasmic RNA. Each dot represents bulk RNA extracted from one replicate (n=two biological pools, each point represents n=three technical replicates). The first replicate includes five sham mice and six mTBI mice, and the second replicate includes four sham mice and four mTBI mice. FIG. 9H shows table summarizing the expression of all complement proteins from the nuclear RNA-seq data and cytoplasmic RNA qPCR.

FIGS. 10A-10G depict that single-nucleus RNA sequencing shows expression gradients within thalamic GABAergic neurons but few differences between sham and mTBI mice. FIG. 10A shows UMAP of all profiled nuclei (sham and mTBI), colored by expression of Slc17a6 (left) and Slc17a7 (right), rendered with imputation. Nuclei within the dashed circle were selected to subcluster for GABAergic neurons. FIG. 10B shows UMAP projection of the GABAergic neurons from A) colored by new subclusters. FIG. 10C shows percentage of each subcluster in the total number of GABAergic neuron nuclei in nine sham mice and ten mTBI mice. Each point represents one of the two biological replicates (rep 1: n=five sham mice, n=six mTBI mice, rep 2: n=four sham mice, n=four mTBI mice). FIG. 10D shows (Top) UMAP projections colored by Ecel1 and Spp1 expression, with imputation. The “overlap” panel combines the two panels to the left, nuclei with strong overlap between the two genes. (Bottom) Same as top, with Sst and Pvalb. Grey circles represent nuclei with no expression. FIG. 10E shows violin plots of Slc17a7, Gad2, Pvalb and Sst from subclusters in (FIG. 10B). FIG. 10F shows heatmap of several genes related to neuronal function in the GABAergic neuronal subclusters. Broad categories for each gene are annotated on the right. Color represents scaled expression, normalized to the mean of all subclusters. Subclusters vary in their expression of adherence and guidance molecules, including genes in the cadherin (Cdh12, Cdh13, Cdh18) and protocadherin families, semaphorin genes (Sema5b, Sema3e) and the ephrin receptor family genes (Epha5, Epha6), related to axonal guidance and growth. Subclusters also varied in their expression of glutamate receptors (Gria1, Grin2a, Grik4), extracellular matrix proteins (Col12a1, Col25a1), and cell signaling molecules such as Nrg1, an epidermal growth factor family member which is thought to regulate Pvalb positive neurons. FIG. 10G shows volcano plot of genes differentially expressed between sham and mTBI mice across all GABAergic neurons. Grey lines designate significance cutoff criteria. A select number of mitochondrial genes are labeled.

FIGS. 11A-11B show that the injured cortex and functionally connected thalamus show chronic inflammation and neuron loss four months after mTBI. FIG. 11A shows close-up images of S1 (top), VB and nRT (middle), and confocal images of nRT (bottom), stained for C1q, NeuN, GFAP, and IBA1. Injury site in the right S1 cortex is marked by an asterisk. Arrow in nRT indicates location of confocal image. Scale bars, 300 μm (top/middle) and 20 μm (bottom). FIG. 11B shows quantification of fluorescence ratios between ipsilateral and contralateral regions in sham and TBI mice. Data represent all points from min to max, with a Mann-Whitney test and α=0.05 (*p<0.05, **p<0.01). Analysis includes between four and six mice per group (n=one to three sections per mouse, one image per region).

FIGS. 12A-12C depict that C1q−/− mice show reduced inflammation and neuron loss three weeks after mTBI. FIG. 12A shows representative coronal brain sections from TBI C1q−/− mice stained for GFAP, IBA1, and NeuN. Injury site in the right S1 cortex is marked by an asterisk. Scale bars, 1 mm. FIG. 12B shows close-up images of S1 (top), VB and nRT (bottom) stained for GFAP, IBA1, and NeuN. Injury site in the right S1 cortex is marked by an asterisk. Scale bars, 500 μm. FIG. 12C shows quantification of fluorescence ratios between ipsilateral and contralateral regions and nRT neuron counts in sham and TBI C1q−/− mice. Data represent all points from min to max, with a Mann-Whitney test and α=0.05 (*p<0.05, **p<0.01). Analysis includes between four and six mice per group (n=one to three sections per mouse, one image per region).

FIGS. 13A-13D shows that plasma and brain PK/PD show presence of free drug and reduced C1q in anti-C1q drug-treated sham and TBI mice. FIG. 13A shows plasma levels of free drug, C1q-free, and C1q-total were measured using sandwich ELISAs after TBI and sham mice were treated with two doses of 100 mg/kg anti-C1q or isotype control antibodies. Dotted line shows lower limit of quantification. FIGS. 13B-13D show levels of free drug (FIG. 13B), C1q-free (FIG. 13C), and C1q-total (FIG. 13D) were measured in brain lysates in the ipsilateral (top) and contralateral (bottom) sides using sandwich ELISAs. Naïve mice were negative controls. Dotted line shows lower limit of quantification. Data represent all points from min to max, with a Mann-Whitney test between TBI control and TBI anti-C1q, and α=0.05 (*p<0.05, **p<0.01, ***p<0.001, ****p<0.0001). Analysis includes between three and 15 mice per group.

FIGS. 14A-14D show that mice with mTBI have spontaneous seizure-like events in the theta to alpha frequency range that are time-locked with thalamic bursting four weeks after mTBI. FIG. 14A show diagram of recording locations for in vivo experiments. Left, ECoG recording sites and mTBI location are shown on the mouse skull. Right, approximate location of tungsten depth electrodes implanted unilaterally in the nRT. FIG. 14B show representative ECoG traces from cortical recording sites and multi-unit traces from nRT showing a spontaneous seizure-like event. FIG. 14C show power spectral analysis showing the average power across different frequency bands in the first 15 minutes of baseline ECoG signal from the ipsilateral S1 cortex in sham and TBI mice. FIG. 14D show periodogram showing the power across frequencies taken from the first 15 minutes of baseline ECoG signal from the ipsilateral S1 cortex in a representative sham and TBI mouse. Data represent mean±SEM analyzed with a Mann-Whitney test and α=0.05 (*p<0.05, **p<0.01). Analysis includes between 12 and 14 mice per group.

FIGS. 15A-15D show that anti-C1q antibody has chronic disease-modifying effects on ECoG power in mice with mTBI. FIG. 15A shows example spectrograms (top) and histograms (bottom) from a control-treated mouse (left) and antibody-treated mouse (right) showing power across different frequency bands 2.5 months post-TBI, which was four weeks after the treatment ended. Power bands are sampled every 30 minutes. FIG. 15B shows cumulative distribution functions for control-treated and antibody-treated cohorts sampled across different frequency bands in the first day post-TBI. We sampled 48 points from the first 24 hours within the start of each recording. FIG. 15C shows same as B, but at three weeks post-TBI. We sampled 232 points between 15.25-20.1 days from the start of each recording. FIG. 15D shows same as B, but at 9-15 weeks post-TBI. We sampled 296 points between 104.6 to 110 days from the start of each recording. Data represent all mice recorded, even if they died before treatment ended. One control-treated mouse and one antibody-treated mouse died within three weeks post-TBI, two control-treated mice died within six weeks post-TBI, and the remaining mice were recorded for at least nine weeks post-TBI. At 24 hours, n=seven control-treated mice, seven antibody-treated mice. At three weeks, n=seven control-treated mice, seven antibody-treated mice. At 9-15 weeks n=six control-treated mice, four antibody-treated mice. Delta=1-4 Hz, theta=5-8 Hz, alpha=9-12 Hz, sigma=13-15 Hz, beta=16-30 Hz, gamma=31-50 Hz. ns=p>0.05.

FIGS. 16A-16D show anti-C1q antibody restores sleep spindle reduction three weeks after mTBI. FIG. 16A shows representative ECoG recordings from a sham and mTBI mouse three weeks post-mTBI. Traces represent the band-pass (BP) filtered ECoG. Horizontal lines: show the detected spindles. Arrows indicate epileptic spikes. FIG. 16B shows same as FIG. 16A from mTBI mice treated with an isotype control or the anti-C1q antibody. FIG. 16C shows ratio of sleep spindles in ipsilateral ECoG to sleep spindles in contralateral ECoG detected within a 12 hour window. Data represent mean±SEM analyzed with a Mann-Whitney Rank Sum test with α=0.05 (*p<0.05, **p<0.01). Analysis includes n=six sham mice, n=nine mTBI mice (left); n=seven control-treated mTBI mice, n=seven antibody-treated mTBI mice (right). FIG. 16D shows frequency, normalized amplitude and duration of sleep spindles in contralateral and ipsilateral ECoG from the mice in (FIG. 16C). Data represent mean±SEM analyzed with a Kruskal-Wallis One Way Analysis of Variance on Ranks, all pairwise multiple comparison procedures (Holm-Sidak method), α=0.05 (*p<0.05, **p<0.01). Gray lines represent contralateral and ipsilateral data for each mouse.

FIGS. 17A-17E show anti-C1q antibody reduces focal epileptic spikes that develop three weeks after mTBI. FIG. 17A shows representative ECoG recordings from a sham and mTBI mouse three weeks post-mTBI. Horizontal dashed lines represent the spike detection threshold. Vertical red lines indicate detected spikes. FIG. 17BA shows same as FIG. 17A from mTBI mice treated with an isotype control or the anti-C1q antibody. Traces in A-B are from episodes of NREM sleep. FIG. 17C shows number of epileptic spikes detected within a 12 hour window. Data represent mean±SEM analyzed with a Mann-Whitney Rank sum test (*p<0.05, **p<0.01). Inset: an average epileptic spike from the mTBI mouse shown in (B) (n=592 spikes; mean (black)±SD (grey). Analysis includes n=six sham mice, n=nine mTBI mice (left); n=seven control-treated mTBI mice, n=six antibody-treated mTBI mice. FIGS. 17D-17E shows number of epileptic spikes versus the ratio of sleep spindles from the mice in FIG. 17C. Individual points represent each mouse and error bars represent mean±SEM across both axes.

FIG. 18 shows sleep spindle detection. Simultaneously recorded ECoG signals (black) from the peri-mTBI cortex and the contralateral cortex were band-pass filtered 8-15 Hz (gray traces), and a sleep spindle detection threshold (Thr.) was applied. The depicted recording is from an anti-C1q treated mTBI mouse three weeks post-mTBI (same mouse as in FIG. 17B).

### DETAILED DESCRIPTION

This description is not to be taken in a limiting sense, but is made merely for the purpose of illustrating the general principles of the invention. The section titles and overall organization of the present detailed description are for the purpose of convenience only and are not intended to limit the present invention.

It is estimated that the chance of having epilepsy during a lifetime of 80 years is about 3%. In about 30% of cases, there is an identifiable injury to the brain that triggered the development of epilepsy (symptomatic epilepsies). Another 30% of patients have presumed symptomatic epilepsy, in which the cause has not been identified. Brain insults such as traumatic brain injury (TBI), stroke, status epilepticus and infection/inflammation are some of the causes of acquired epilepsy, which usually occurs after a latent period and is often progressive (i.e., the seizures become more frequent and severe over time). Epileptic seizures may also occur in recovering patients as a consequence of brain surgery. In addition, for approximately 1% of the population, epileptic seizures are spontaneous without any obvious reason or any other neurological abnormalities. Such spontaneous epilepsies are named idiopathic epilepsies and are assumed to be of genetic origin.

Epilepsy is not a specific disease, or even a single syndrome, but rather a broad category of symptom complexes arising from any number of disordered brain functions that may be secondary to a variety of pathologic processes. The terms convulsive disorder, seizure disorder, and cerebral seizures are used synonymously with epilepsy, as they all refer to recurrent paroxysmal episodes of brain dysfunction manifested by stereotyped alterations in behavior. An epileptic seizure is known as a sudden change in behavior that is the consequence of electrical hypersynchronization of neuronal networks involving the cortex. An epileptic seizure can also be a natural response of the normal brain to transient disturbance in function, and therefore, not necessarily an indication of an epileptic disorder. Such seizures are often referred to as provoked, acute symptomatic, or reactive.

Some genes coding for protein subunits of voltage-gated and ligand-gated ion channels have been associated with different forms of epilepsy and infantile seizure syndromes. Patients with uncontrolled seizures experience significant morbidity and mortality.

In epilepsy, the brain has become permanently altered pathophysiologically or structurally leading to abnormal, hypersynchronous neuronal firing. Progressive brain damage can occur as a consequence of repeated seizures. For example, a progressive decrease in hippocampal volume over time as a function of seizure number has been reported. Several clinical and experimental data have implicated the failure of blood-brain barrier (BBB) function in triggering chronic or acute seizures.

Over forty types of epileptic seizures have been characterized and these are divided into generalized (seizure onset in both hemispheres of the brain) and partial (focal; seizure onset in one part of the brain). Generalized seizures are further divided into absence, myoclonic, atonic, and tonic seizures, while partial seizures are subdivided into simple and complex. Partial seizures account for approximately sixty percent of all adult cases and temporal lobe epilepsy (TLE) is the most common form of partial seizure. TLE patients often have a history of early risk factors such as febrile seizures, status epilepticus, and infection. A seizure-free period may be present before uncontrolled partial seizures begin. There are some progressive features such as increasing seizure frequency and cognitive decline.

The etiology of epilepsy remains enigmatic. However, there is evidence that suggests activation of immune pathways plays a role in human epilepsy and that this inflammatory response contributes both to the generation and recurrence of seizures and to seizure-related neuronal damage. Astrocytes are known to contribute to the inflammatory environment of the CNS by producing a wide range of immunologically relevant molecules. They can express class II major histocompatibility complex antigens, and produce a variety of chemokines and cytokines. Such immune factors may also activate microglia.

For example, in temporal lobe epilepsy (TLE) patients, there is evidence of microglial activation within the hippocampus, providing evidence of an activated immune response. Nuclear factor kappa B overexpression has been shown in reactive astrocytes and surviving neurons in human hippocampal sclerosis specimens. In addition, there is prominent and persistent activation of the IL-1B system involving both activated glial cells and neuronal cells. In contrast, only a few cells of adaptive immunity (CD3/CD8-positive T lymphocytes) have been detected in human mesial TLE specimens. Furthermore, the activation of inflammatory pathways in human TLE is also supported by gene expression profile analysis. A recent study demonstrated differential correlation of key inflammatory factor expression and seizure frequency in patients with pharmacoresistant mesial TLE. Toll-like receptor 4 (TLR4—a key trigger of inflammation previously shown to induce the transcription of several cytokines in a TLE animal model) gene expression correlated directly, whereas activating transcription factor 3 (a negative regulator of TLR4) and IL-8 expressions correlated inversely with seizure frequency.

Interactions between dysregulated persistent inflammation, blood-brain barrier damage, and uncontrolled seizures can create a self-perpetuating cycle causing uncontrolled inflammation that triggers progression of different epileptic disorders, including TLE. For example, one key inflammatory mediator is the complement system. The complement system is a protein cascade involved in the immune response consisting of around 30 fluid-phase and cell membrane associated proteins. The activation products of the cascade contribute to the production of other inflammatory mediators, and can therefore promote tissue injury at sites of inflammation. Even though the synthesis of components of the complement system occurs predominantly in the liver, both glia and neurons can express these inflammatory mediators in pathological conditions. C3a and C5a are the most potent proinflammatory molecules produced in response to complement activation. The initiating molecule of the classical complement cascade, C1q, recognizes synapses of neurons under stress. Activation of C1q leads to deposition of downstream complement components C4b and C3b on the synapse surface—leading to recognition by immune cells and physical elimination of the synapse. Activation of the complement system also leads to formation of the membrane attack complex, which damages or lyses target cells by forming a pore in the phospholipid bilayer. Neurons are particularly susceptible to complement-mediated damage. In addition, while complement factors might invade the brain via a leaky BBB, some of the increased expression is likely to originate from activated glial cells. Interestingly, sequential infusion of individual proteins of the membrane attack pathway (C5b6, C7, C8, and C9) into the hippocampus of awake, freely moving rats induces both behavioral and electrographic seizures as well as cytotoxicity, suggesting a role for the complement system in epileptogenesis.

In addition, traumatic brain injury (TBI) affects about 69 million people worldwide every year and can lead to cognitive dysfunction, difficulty with sensory processing, sleep disruption, and as mentioned above, the development of epilepsy. Most of these adverse health outcomes develop months or years after TBI and are caused by indirect secondary injuries that result in long-term consequences of the initial impact.

While TBI acutely disrupts the cortex, most TBI-related disabilities reflect secondary injuries that accrue over time. The thalamus is a likely site of secondary damage because of its reciprocal connections with the cortex. Using a mouse model of mild cortical injury that does not directly damage subcortical structures (mTBI), we found a chronic increase in C1q expression specifically in the corticothalamic circuit. Increased C1q expression co-localized with neuron loss and chronic inflammation, and correlated with disruption in sleep spindles and emergence of epileptic activities. Blocking C1q counteracted most of these outcomes, showing that C1q is a disease modifier in mTBI. Single-nucleus RNA sequencing demonstrated that microglia are the source of thalamic C1q, which likely acts on a subset of oligodendrocytes and astrocytes. The cortex is often the site of primary injury because it sits directly beneath the skull, and is an integrated part of many larger circuits, including the cortico-thalamo-cortical loop. This circuit is important for sensory processing, attention, cognition, and sleep, all of which can be impaired by TBI. The thalamus itself, though not acutely injured in TBI, experiences secondary injury, presumably because of its long-range reciprocal connections with the cerebral cortex. Structural changes in the thalamus have been implicated in a number of long-term TBI-related health outcomes, including fatigue and cognitive dysfunction, and patients with TBI display secondary and chronic neurodegeneration and inflammation in thalamic nuclei.

Chronic neuroinflammation is a common feature of secondary injury sites. But most attempts to improve post-TBI cognitive outcomes with broad anti-inflammatory agents have failed, likely because there are many inflammatory pathways that play both protective and pathogenic roles at different times. A potential mediator of post-TBI inflammation and injury is the complement pathway, which is activated in the peri-injury area of brain lesions in both humans and rodents. Complement activation contributes to inflammation and neurotoxicity in central nervous system injury and is increased in human brains afflicted with injury, epilepsy, and Alzheimer's disease. Aberrant activation of C1q, the initiating molecule of the classical complement cascade, can trigger elimination of functioning synapses and contribute to the progression of neurodegenerative disease. On the other hand, C1q is involved in normal synapse pruning during development and the complement system plays an important part in brain homeostasis by clearing cellular debris and protecting the central nervous system from infection.

Provided herein is the discovery of the role of the C1q pathway in post-TBI secondary injury to the corticothalamic circuit in a mechanistically tractable and highly reproducible mouse model of mild cortical injury. This model identifies factors such as therapeutic windows, inflammatory phenotypes, and degree of secondary damage, which support targeted approaches in the treatment of post-TBI outcomes.

One powerful tool we employ to study the entire somatosensory corticothalamic circuit after TBI is chronic ECoG recordings which are used specifically to study the progression of post-traumatic epileptogenesis and changes in cortical rhythms up to four months post TBI. Using such electrophysiological approaches at the cellular and circuit levels, we show that TBI alters the synaptic properties of thalamic reticular nucleus (nRT) neurons and is associated with increased C1q accumulation that might mediate pathological states in the corticothalamic circuit, including increased broadband activity.

### The nRT as a Locus of Long-Term, Secondary Impairments Post-TBI

Previous observations of severe head injury show neurodegeneration in the human nRT (42). Our studies show that even mild cortical injury can lead to neuronal loss in the nRT three weeks after the injury. A cause for this neurodegeneration could be the loss of cortical inputs causing excitotoxicity in the nRT, which may be a vulnerable brain region due to the high density of axonal afferents from the cortex (42). Our RNA sequencing results also identify increased expression of genes related to mitochondrial function in mTBI thalamic tissue across all GABAergic neuron subclusters. This observation points to mitochondria-mediated cell death as another potential mechanism of post-TBI neurodegeneration.

The loss of neurons in the nRT could explain some of the synaptic changes in this area. In particular, three weeks post-TBI, the frequency of IPSCs was reduced in nRT neurons. In many microcircuits, reduced inhibition on GABAergic neurons results in a net increase in inhibition. By contrast, loss of GABAergic inhibition in the nRT results in corticothalamic circuit hyperexcitability, and can even elicit epileptiform activity. Indeed, intra-nRT GABAergic connections are important for coordinating inhibitory output to the excitatory thalamic nuclei and controlling oscillatory thalamic activity, and their loss is deleterious to the corticothalamic circuit. The death of GABAergic neurons in the nRT may contribute to reduced intra-nRT inhibition. This reduced inhibition could cause a loss of feed-forward GABAergic inhibition, which may contribute to increased seizure susceptibility, and increased likelihood of developing post-traumatic epileptic activities.

Deficits were also observed in nRT EPSCs, in particular lower frequency and amplitude and slower kinetics. These alterations are similar to the findings from a mouse model of epilepsy that lacks GluA4 AMPA receptors at the cortico-nRT glutamatergic synapse. This defect results in loss of feed-forward inhibition in the thalamus, and epileptic activities. Alterations to the nRT EPSCs thus appear to contribute to corticothalamic circuit hypersynchrony and seizures, but likely result from a loss of cortical glutamatergic inputs to the nRT after TBI.

Given that the changes we found in the corticothalamic circuit, and the nRT in particular, have been implicated in epileptic activities and cognitive deficits, these results pinpoint this circuit as a novel potential target for treating long-term TBI outcomes. Of particular interest, sleep spindles play a major role in cognitive functions. Our finding pinpoints C1q as a target for treating sleep spindles and preventing epileptic spikes after mTBI.

Unlike nRT neurons, cortical neurons, such as layer-5 pyramidal neurons and GABAergic fast-spiking interneurons, were not altered by mild TBI (mTBI) at chronic time points. These observations suggest the presence of homeostatic mechanisms that restore or reduce chronic hyperexcitability after TBI in the cortex. They also confirm that at least certain long-term outcomes of TBI must result from nRT dysfunction rather than simply from damage to the cortex. In this regard, it is interesting to see that while cortical neurons appear to have normal excitability and synaptic function at the chronic phase, the ECoG shows ‘local’ deficits in sleep spindles and epileptic spikes. This observation is in agreement with previous magnetoencephalography studies in humans with mild TBI (mTBI), EEG studies in humans with severe TBI, and EEG studies from rats with severe TBI, which observed increased delta activity at early time points post-TBI. In normal conditions, delta activity is associated with slow wave sleep, quiet wakefulness, and higher cognitive function. In cases of injury, delta waves are associated with a white matter lesion). Therefore, the major long-term impact of mild TBI (mTBI) is in the thalamic end of the cortico-thalamo-cortical loop. Given the emerging role of the nRT in generating local sleep spindles in the sensory cortex, the secondary damage to the nRT may be responsible, at least in part, for the ‘local’ loss of sleep spindles in the cortex.

### Chronic C1q as a Disease Modifier

C1q has a well-documented role in normal brain function such as synaptic pruning during development, as well as its involvement in several neurological diseases, including severe TBI. In addition, C1q expression was highly increased in the corticothalamic circuit for up to four months after TBI. The study disclosed herein focuses on the corticothalamic circuit, and provides the first functional characterization of the corticothalamic circuit after mTBI using electrophysiological recordings and the first demonstration that the loss of sleep spindles and development of epileptic spikes after mTBI involve the C1q pathway.

Although the mild TBI (mTBI) mice did not develop chronic GTCSs to determine if blocking C1q had an anti-seizure effect, many other protective effects of the anti-C1q antibody were observed, including reduced inflammation and neurodegeneration, and restoration of altered cortical states post-TBI, such as protection against sleep spindle disruption and epileptic spikes. Based on these observations and previous literature implicating differences between protective and harmful inflammatory cell types, C1q plays different roles but at different stages of pathology. At the time of the injury, C1q aids with the formation of the glial scar that limits the size of the injury within the primary site of the cortex. However, at the chronic phase, C1q increase promotes chronic inflammation and secondary neurodegeneration in the nRT.

The cortex also exhibits an increase in C1q, but it does not appear to have a damaging role at this site or in this time, or may play a counterbalancing initial protective role since, unlike in the thalamus, the neuronal physiology is similar in the cortex of sham and TBI mice at chronic time points. These findings suggest the existence of a time window during which the anti-C1q treatment can prevent secondary damage to the thalamus without impairing homeostatic recovery at the cortex.

Our RNA sequencing results suggest that microglia are the main source of C1q, and astrocytes and oligodendrocytes of C4b, findings which could point to additional cell-specific therapeutic targets both upstream (microglia) and downstream (astrocytes and oligodendrocytes) of the C1q molecule itself. C4 also appears to mediate injury after severe TBI, as shown by reduced motor deficits in C4−/− mice. The lack of Hc expression in our sequencing data suggests that the mechanism of nRT neuron death is not membrane attack complex-mediated lysis, although the mechanism cannot be determined by sequencing approaches alone.

The study disclosed herein pinpoints C1q as a disease modifier that could be targeted for treating devastating outcomes of TBI within a certain time window (in this study, beginning treatment 24 hours post-injury). Thalamic C1q might also serve as a biomarker to help identify those individuals likely to develop long-term, secondary injuries. This study is the first to perform electrophysiological recordings in both the cortex and the thalamus at a chronic time point after mTBI, and to identify neuronal death and IPSC reduction in the nRT as chronic outcomes of cortical injury. In addition, by showing the physiological chronic outcomes of mTBI in the nRT, we identify the nRT as a novel target for treatments of post-TBI outcomes such as altered sensory processing, sleep disruption, and epilepsy.

Despite these studies regarding the roles of different immune pathways, TLE, and post-TBI secondary injury (e.g. TBI-induced epilepsy, etc.), there is a need in the art for new compositions and methods for preventing and treating epilepsy and its associated progression. Accordingly, inhibition of early complement activation pathways may be a promising therapeutic strategy for epilepsy, e.g., using anti-C1q, anti-C1r, and anti-C1s antibodies that inhibit the early stages of complement activation, including the complement activation pathway. The antibodies may be monoclonal antibodies, chimeric antibodies, humanized antibodies, antibody fragments, and/or antibody derivatives.

Neutralizing the activity of complement factors such as C1q, C1r, or C1s inhibits classical complement activity, and slow or prevent epilepsy, such as an idiopathic generalized epilepsy, idiopathic partial epilepsy, symptomatic generalized epilepsy or symptomatic partial epilepsy. Methods related to neutralizing complement factors such as C1q, C1r, or C1s in epilepsy, such as an idiopathic generalized epilepsy, idiopathic partial epilepsy, symptomatic generalized epilepsy or symptomatic partial epilepsy are disclosed herein.

All sequences mentioned in the present disclosure are incorporated by reference from U.S. Pat. No. 10,316,081, U.S. patent application Ser. No. 14/890,811, U.S. Pat. Nos. 8,877,197, 9,708,394, U.S. patent application Ser. No. 15/360,549, U.S. Pat. Nos. 9,562,106, 10,450,382, 10,457,745, International Patent Application No. PCT/US2018/022462 each of which is hereby incorporated by reference for the antibodies and related compositions that it discloses.

In certain aspects, disclosed herein is a method of preventing, reducing risk of developing, or treating epilepsy, such as an idiopathic generalized epilepsy, idiopathic partial epilepsy, symptomatic generalized epilepsy or symptomatic partial epilepsy, comprising administering to a subject an inhibitor of the complement pathway.

Disclosed herein is a method of inhibiting epilepsy, such as an idiopathic generalized epilepsy, idiopathic partial epilepsy, symptomatic generalized epilepsy or symptomatic partial epilepsy, comprising administering to a patient an antibody, such as an anti-C1q antibody, an anti-C1r antibody, or an anti-C1s antibody. In certain preferred embodiments, the antibody binds to C1q, C1r, or C1s and inhibits complement activation. The antibody may be a monoclonal antibody, a chimeric antibody, a humanized antibody, an antibody fragment thereof, and/or an antibody derivative thereof.

Full-length antibodies may be prepared by the use of recombinant DNA engineering techniques. Such engineered versions include those created, for example, from natural antibody variable regions by insertions, deletions or changes in or to the amino acid sequences of the natural antibodies. Particular examples of this type include those engineered variable region domains containing at least one CDR and optionally one or more framework amino acids from one antibody and the remainder of the variable region domain from a second antibody. The DNA encoding the antibody may be prepared by deleting all but the desired portion of the DNA that encodes the full-length antibody. DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the complementarity determining regions (CDRs) derived substantially or exclusively from the corresponding human antibody regions and DNA encoding CDRs derived substantially or exclusively from a mammal other than a human.

Suitable sources of DNA molecules that encode antibodies include cells, such as hybridomas, that express the full-length antibody. For example, the antibody may be isolated from a host cell that expresses an expression vector that encodes the heavy and/or light chain of the antibody.

Antibody fragments and/or antibody derivatives may also be prepared by the use of recombinant DNA engineering techniques involving the manipulation and re-expression of DNA encoding antibody variable and constant regions. Standard molecular biology techniques may be used to modify, add or delete further amino acids or domains as desired. Any alterations to the variable or constant regions are still encompassed by the terms ‘variable’ and ‘constant’ regions as used herein. In some instances, PCR is used to generate an antibody fragment by introducing a stop codon immediately following the codon encoding the interchain cysteine of C_{H}1, such that translation of the C_{H}1 domain stops at the interchain cysteine. Methods for designing suitable PCR primers are well known in the art and the sequences of antibody C_{H}1 domains are readily available. In some embodiments, stop codons may be introduced using site-directed mutagenesis techniques.

An antibody of the present disclosure may be derived from any antibody isotype (“class”) including for example IgG, IgM, IgA, IgD and IgE and subclasses thereof, including for example IgG1, IgG2, IgG3 and IgG4. In certain preferred embodiments, the heavy and light chains of the antibody are from IgG. The heavy and/or light chains of the antibody may be from murine IgG or human IgG. In certain other preferred embodiments, the heavy and/or light chains of the antibody are from human IgG1. In still other preferred embodiments, the heavy and/or light chains of the antibody are from human IgG4.

In some embodiments, the inhibitor is an antibody, such as an anti-C1q antibody, an anti-C1r antibody, or an anti-C1s antibody. The anti-C1q antibody may inhibit the interaction between C1q and an autoantibody, or between C1q and C1r, or between C1q and C1s. The anti-C1r antibody may inhibit the interaction between C1r and C1q, or between C1r and C1s. The anti-C1r antibody may inhibit the catalytic activity of C1r, or the anti-C1r antibody may inhibit the processing of pro-C1r to an active protease. The anti-C1s antibody may inhibit the interaction between C1s and C1q, or between C1s and C1r, or between C1s and C2 or C4, or the anti-C1s antibody may inhibit the catalytic activity of C1s, or it may inhibit the processing of pro-C1s to an active protease. In some instances, the anti-C1q, anti-C1r, or anti-C1s antibody causes clearance of C1q, C1r or C1s from the circulation or a tissue.

The antibody disclosed herein may be a monoclonal antibody, e.g., that binds mammalian C1q, C1r, or C1s, preferably human C1q, C1r, or C1s. The antibody may be a mouse antibody, a human antibody, a humanized antibody, a chimeric antibody, an antibody fragment, or an antibody derivative thereof. The antibodies disclosed herein may also cross the blood brain barrier (BBB). The antibody may activate a BBB receptor-mediated transport system, such as a system that utilizes the insulin receptor, transferrin receptor, leptin receptor, LDL receptor, or IGF receptor. The antibody can be a chimeric antibody with sufficient human sequence that is suitable for administration to a human. The antibody can be glycosylated or nonglycosylated; in some embodiments, the antibody is glycosylated, e.g., in a glycosylation pattern produced by post-translational modification in a CHO cell. In some embodiments, the antibodies are produced in E. coli.

The antibody may be a bispecific antibody, recognizing a first and a second antigen, e.g., the first antigen is selected from C1q, C1r, and C1s and/or the second antigen is an antigen that allows the antibody to cross the blood-brain-barrier, such as an antigen selected from transferrin receptor (TR), insulin receptor (HIR), Insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, or ANG1005.

An antibody of the present disclosure may bind to and inhibit a biological activity of C1q, C1r, C1s, or C1. For example, (1) C1q binding to an autoantibody, (2) C1q binding to C1r, (3) C1q binding to C1s, (4) C1q binding to phosphatidylserine, (5) C1q binding to pentraxin-3, (6) C1q binding to C-reactive protein (CRP), (7) C1q binding to globular C1q receptor (gC1qR), (8) C1q binding to complement receptor 1 (CR1), (9) C1q binding to B-amyloid, or (10) C1q binding to calreticulin. In other embodiments, the biological activity of C1q is (1) activation of the classical complement activation pathway, (2) activation of antibody and complement dependent cytotoxicity, (3) CH50 hemolysis, (4) synapse loss, (5) B-cell antibody production, (6) dendritic cell maturation, (7) T-cell proliferation, (8) cytokine production (9) microglia activation, (10) Arthus reaction, (11) phagocytosis of synapses or nerve endings or (12) activation of complement receptor 3 (CR3/C3) expressing cells.

In some embodiments, CH50 hemolysis comprises human, mouse, and/or rat CH50 hemolysis. In some embodiments, the antibody is capable of neutralizing from at least about 50%, to at least about 95% of CH50 hemolysis. The antibody may also be capable of neutralizing at least 50% of CH50 hemolysis at a dose of less than 150 ng/ml, less than 100 ng/ml, less than 50 ng/ml, or less than 20 ng/ml.

Other in vitro assays to measure complement activity include ELISA assays for the measurement of split products of complement components or complexes that form during complement activation. Complement activation via the classical pathway can be measured by following the levels of C4d and C4 in the serum. Activation of the alternative pathway can be measured in an ELISA by assessing the levels of Bb or C3bBbP complexes in circulation. An in vitro antibody-mediated complement activation assay may also be used to evaluate inhibition of C3a production.

An antibody of the present disclosure may be a monoclonal antibody, a polyclonal antibody, a recombinant antibody, a humanized antibody, a chimeric antibody, a multispecific antibody, an antibody fragment thereof, or a derivative thereof.

The antibodies of the present disclosure may also be an antibody fragment, such as a Fab fragment, a Fab′ fragment, a F(ab′)₂ fragment, a Fv fragment, a diabody, or a single chain antibody molecule.

Disclosed herein are methods of administering to the subject a second agent, such as a second inhibitor. In some embodiments, the second inhibitor may be an antibody (e.g., an anti-C1q antibody, an anti-C1r antibody, or an anti-C1s antibody). In other embodiments, the second inhibitor may be an inhibitor of antibody-dependent cellular cytotoxicity, alternative complement activation pathway; and/or an inhibitor of the interaction between the autoantibody and an autoantigen.

In some embodiments, a method is provided of determining a subject's risk of developing epilepsy, such as an idiopathic generalized epilepsy, idiopathic partial epilepsy, symptomatic generalized epilepsy or symptomatic partial epilepsy, comprising: (a) administering an antibody to the subject (i.e. an anti-C1q, anti-C1r, or anti-C1s antibody), wherein the antibody is coupled to a detectable label; (b) detecting the detectable label to measure the amount or location of C1q, C1r, or C1s in the subject; and (c) comparing the amount or location of one or more of C1q, C1r, or C1s to a reference, wherein the risk of developing epilepsy, such as an idiopathic generalized epilepsy, idiopathic partial epilepsy, symptomatic generalized epilepsy or symptomatic partial epilepsy is characterized based on a the comparison of the amount or location of one or more of C1q, C1r, or C1s to the reference. The detectable label may comprise a nucleic acid, oligonucleotide, enzyme, radioactive isotope, biotin or a fluorescent label. In some instances, the antibody may be labeled with a coenzyme such as biotin using the process of biotinylation. When biotin is used as a label, the detection of the antibody is accomplished by addition of a protein such as avidin or its bacterial counterpart streptavidin, either of which can be bound to a detectable marker such as the aforementioned dye, a fluorescent marker such as fluorescein, a radioactive isotope or an enzyme such as peroxidase. In some embodiments, the antibody is an antibody fragment (e.g., Fab, Fab′-SH, Fv, scFv, or F(ab′)₂ fragments).

The antibodies disclosed herein may also be coupled to a labeling group, e.g., an radioisotope, radionuclide, an enzymatic group, biotinyl group, a nucleic acid, oligonucleotide, enzyme, or a fluorescent label. A labeling group may be coupled to the antibody via a spacer arm of any suitable length to reduce potential steric hindrance. Various methods for labeling proteins are known in the art and can be used to prepare such labeled antibodies.

Various routes of administration are contemplated. Such methods of administration include but are not limited to, topical, parenteral, subcutaneous, intraperitoneal, intrapulmonary, intrathecal, intranasal, and intralesional administration. Parenteral infusions include intramuscular, intravenous, intraarterial, intraperitoneal, or subcutaneous administration. For treatment of central nervous system conditions, the antibody may be adapted to cross the blood-brain barrier following a non-invasive peripheral route of administration such as intravenous intramuscular, subcutaneous, intraperitoneal, or even oral administration.

Suitable antibodies include antibodies that bind to complement component C1q, C1r, or C1s. Such antibodies include monoclonal antibodies chimeric antibodies, humanized antibodies, antibody fragments, and/or an antibody derivative thereof.

Preferred antibodies are monoclonal antibodies, which can be raised by immunizing rodents (e.g., mice, rats, hamsters and guinea pigs) with either (1) the native complement component (e.g., C1q, C1r, or C1s) derived from enzymatic digestion of a purified complement component from human plasma or serum, or (2) a recombinant complement component, or its derived fragment, expressed by either eukaryotic or prokaryotic systems. Other animals can be used for immunization, e.g., non-human primates, transgenic mice expressing human immunoglobulins, and severe combined immunodeficient (SCID) mice transplanted with human B-lymphocytes.

Polyclonal and monoclonal antibodies are naturally generated as immunoglobulin (Ig) molecules in the immune system's response to a pathogen. A dominating format with a concentration of 8 mg/ml in human serum, the ˜150-kDa IgG1 molecule is composed of two identical ˜50-kDa heavy chains and two identical ˜25-kDa light chains.

Hybridomas can be generated by conventional procedures by fusing B-lymphocytes from the immunized animals with myeloma cells. In addition, anti-C1q, -C1r, or -C1s antibodies can be generated by screening recombinant single-chain Fv or Fab libraries from human B-lymphocytes in a phage-display system. The specificity of the MAbs to human C1q, C1r, or C1s can be tested by enzyme linked immunosorbent assay (ELISA), Western immunoblotting, or other immunochemical techniques.

The inhibitory activity on complement activation of antibodies identified in the screening process can be assessed by hemolytic assays using either unsensitized rabbit or guinea pig RBCs for the alternative complement pathway, or sensitized chicken or sheep RBCs for the classical complement pathway. Those hybridomas that exhibit an inhibitory activity specific for the classical complement pathway are cloned by limiting dilution. The antibodies are purified for characterization for specificity to human C1q, C1r, or C1s by the assays described above.

Based on the molecular structures of the variable regions of the anti-C1q, -C1r, or -C1s antibodies, molecular modeling and rational molecular design may be used to generate and screen small molecules that mimic the molecular structures of the binding region of the antibodies and inhibit the activities of C1q, C1r, or C1s. These small molecules can be peptides, peptidomimetics, oligonucleotides, or organic compounds. The mimicking molecules can be used as inhibitors of complement activation in inflammatory indications and autoimmune diseases. Alternatively, one can use large-scale screening procedures commonly used in the field to isolate suitable small molecules from libraries of combinatorial compounds.

A suitable dosage of an antibody as disclosed herein may be between 10 and 500 μg/ml of serum. The actual dosage can be determined in clinical trials following the conventional methodology for determining optimal dosages, i.e., administering various dosages and determining which doses provide suitable efficacy without undesirable side-effects.

Before the advent of recombinant DNA technology, proteolytic enzymes (proteases) that cleave polypeptide sequences were used to dissect the structure of antibody molecules and to determine which parts of the molecule are responsible for its various functions. Limited digestion with the protease papain cleaves antibody molecules into three fragments. Two fragments, known as Fab fragments, are identical and contain the antigen-binding activity. The Fab fragments correspond to the two identical arms of the antibody molecule, each of which consists of a complete light chain paired with the V_{H} and C_{H}1 domains of a heavy chain. The other fragment contains no antigen binding activity but was originally observed to crystallize readily, and for this reason was named the Fc fragment (Fragment crystallizable).

A Fab molecule is an artificial ˜50-kDa fragment of the Ig molecule with a heavy chain lacking constant domains C_{H}2 and C_{H}3. Two heterophilic (V_{L}-V_{H} and C_{L}-C_{H}1) domain interactions underlie the two-chain structure of the Fab molecule, which is further stabilized by a disulfide bridge between C_{L} and C_{H}1. Fab and IgG have identical antigen binding sites formed by six complementarity-determining regions (CDRs), three each from V_{L} and V_{H} (LCDR1, LCDR2, LCDR3 and HCDR1, HCDR2, HCDR3). The CDRs define the hypervariable antigen binding site of antibodies. The highest sequence variation is found in LCDR3 and HCDR3, which in natural immune systems are generated by the rearrangement of V_{L} and J_{L} genes or V_{H}, D_{H} and J_{H} genes, respectively. LCDR3 and HCDR3 typically form the core of the antigen binding site. The conserved regions that connect and display the six CDRs are referred to as framework regions. In the three-dimensional structure of the variable domain, the framework regions form a sandwich of two opposing antiparallel β-sheets that are linked by hypervariable CDR loops on the outside and by a conserved disulfide bridge on the inside.

### Definitions

As used herein the specification, “a” or “an” may mean one or more. As used herein in the claim(s), when used in conjunction with the word “comprising”, the words “a” or “an” may mean one or more than one. For example, reference to an “antibody” is a reference from one to many antibodies. As used herein “another” may mean at least a second or more.

As used herein, administration “conjointly” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, antibody fragments so long as they exhibit the desired biological activity, and antibody derivatives.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

The term “agent” as used herein describes any molecule, e.g. protein or pharmaceutical, with the capability of modulating synapse loss, particularly through the complement pathway. Candidate agents also include genetic elements, e.g., anti-sense and RNAi molecules to inhibit C1q expression, and constructs encoding complement inhibitors, e.g., CD 59, and the like. Candidate agents encompass numerous chemical classes, though typically they are organic molecules, including small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally, a plurality of assay mixtures are run in parallel with different agent concentrations to obtain a differential response to the various concentrations. Typically one of these concentrations serves as a negative control, i.e., at zero concentration or below the level of detection.

“Full-length antibodies” are usually heterotetrameric glycoproteins of about 150,000 daltons, comprising two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” molecule or cell is a molecule or a cell that is identified and separated from at least one contaminant molecule or cell with which it is ordinarily associated in the environment in which it was produced. Preferably, the isolated molecule or cell is free of association with all components associated with the production environment. The isolated molecule or cell is in a form other than in the form or setting in which it is found in nature. Isolated molecules therefore are distinguished from molecules existing naturally in cells; isolated cells are distinguished from cells existing naturally in tissues, organs, or individuals. In some embodiments, the isolated molecule is an anti-C1s, anti-C1q, or anti-C1r antibody of the present disclosure. In other embodiments, the isolated cell is a host cell or hybridoma cell producing an anti-C1s, anti-C1q, or anti-C1r antibody of the present disclosure.

An “isolated” antibody is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In certain preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. An isolated antibody includes the antibody in situ within recombinant T-cells since at least one component of the antibody's natural environment will not be present. Ordinarily, however, an isolated polypeptide or antibody will be prepared by a process including at least one purification step.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

As used herein, the term “CDR” or “complementarity determining region” is intended to mean the non-contiguous antigen binding sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

The term “monoclonal antibody” as used herein refers to an antibody obtained from a population of substantially homogeneous antibodies, i.e., the individual antibodies of the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous since they are typically synthesized by hybridoma culture, uncontaminated by other immunoglobulins. The modifier “monoclonal” indicates the character of the antibody as being obtained as a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” and “whole antibody” are used interchangeably to refer to an antibody in its substantially intact form, as opposed to an antibody fragment or antibody derivative. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” or “functional fragments” of antibodies comprises a portion of an intact antibody, preferably the antigen binding and/or the variable region of the intact antibody or the F region of an antibody which retains or has modified FcR binding capability. Examples of antibody fragments include Fab, Fab′, F(ab′)₂ and Fv fragments; diabodies; and linear antibodies (see U.S. Pat. No. 5,641,870, Example 2; Zapata et al., Protein Eng. 8(10):1057-1062 (1995)). Additional examples of antibody fragments include antibody derivatives such as single-chain antibody molecules, monovalent antibodies and multispecific antibodies formed from antibody fragments

An “antibody derivative” is any construct that comprises the antigen binding region of an antibody. Examples of antibody derivatives include single-chain antibody molecules, monovalent antibodies and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments with hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (See, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the VH and VL domains of the two antibodies are present on different polypeptide chains. Diabodies are described in greater detail in, for example, EP 404,097; WO 1993/011161; WO/2009/121948; WO/2014/191493; Hollinger et al., Proc. Nat'l Acad. Sci. USA 90:6444-48 (1993).

As used herein, a “chimeric antibody” refers to an antibody (immunoglobulin) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, NJ, 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or fewer, 9 or fewer, 8 or fewer, 7 or fewer, 6 or fewer, 5 or fewer, 4 or fewer, 3 or fewer, or 2 or fewer. Where pre-existing amino acid changes are present in a VH, preferable those changes occur at only three, two, or one of positions 71H, 73H and 78H; for instance, the amino acid residues at those positions may by 71A, 73T and/or 78A. In some embodiments, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 108 M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated. (See e.g., Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991)

As used herein, an “interaction” between a complement protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

A “blocking” antibody, an “antagonist” antibody, an “inhibitory” antibody, or a “neutralizing” antibody is an antibody that inhibits or reduces one or more biological activities of the antigen it binds, such as interactions with one or more proteins. In some embodiments, blocking antibodies, antagonist antibodies, inhibitory antibodies, or “neutralizing” antibodies substantially or completely inhibit one or more biological activities or interactions of the antigen.

The term “inhibitor” refers to a compound having the ability to inhibit a biological function of a target biomolecule, for example, an mRNA or a protein, whether by decreasing the activity or expression of the target biomolecule. An inhibitor may be an antibody, a small molecule, or a nucleic acid molecule. The term “antagonist” refers to a compound that binds to a receptor, and blocks or dampens the receptor's biological response. The term “inhibitor” may also refer to an “antagonist.”

Antibody “effector functions” refer to those biological activities attributable to the Fc region (a native sequence Fc region or amino acid sequence variant Fc region) of an antibody, and vary with the antibody isotype.

As used herein, the term “affinity” refers to the equilibrium constant for the reversible binding of two agents (e.g., an antibody and an antigen) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. For example, a subject anti-C1s antibody binds specifically to an epitope within a complement C1s protein. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The term “kₒₙ”, as used herein, is intended to refer to the rate constant for association of an antibody to an antigen.

The term “k_{off}”, as used herein, is intended to refer to the rate constant for dissociation of an antibody from the antibody/antigen complex.

The term “K_{D}”, as used herein, is intended to refer to the equilibrium dissociation constant of an antibody-antigen interaction.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

An “isolated” nucleic acid molecule is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acids encoding any polypeptides and antibodies herein that exist naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors useful in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or aralkyl. Not all linkages in a polynucleotide need be identical. The preceding description applies to all polynucleotides referred to herein, including RNA and DNA.

A “gene editing agent” as used herein, is defined as an gene editing agent, representative examples of which include CRISPR-associated nucleases such as Cas9 and Cpfl gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- and/or exo-nucleases. See Schiffer, 2012, J Virol 88(17):8920-8936, hereby incorporated by reference.

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs, shRNAs, or other RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest, such as a complement pathway component like C1q, C1r, and/or C1s. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

“Piwi-interacting RNA (piRNA)” is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

“Aptamers” are oligonucleotide or peptide molecules that bind to a specific target molecule. “Nucleic acid aptamers” are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand.

Preferably, the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™

The term “preventing” is art-recognized, and when used in relation to a condition, such as epilepsy, such as an idiopathic generalized epilepsy, idiopathic partial epilepsy, symptomatic generalized epilepsy or symptomatic partial epilepsy, is well understood in the art, and includes administration of a composition which reduces the frequency or severity, or delays the onset, of one or more symptoms of the medical condition in a subject relative to a subject who does not receive the composition. Similarly, the prevention of epilepsy, such as an idiopathic generalized epilepsy, idiopathic partial epilepsy, symptomatic generalized epilepsy or symptomatic partial epilepsy includes reducing the likelihood that a patient receiving a therapy will develop epilepsy, such as an idiopathic generalized epilepsy, idiopathic partial epilepsy, symptomatic generalized epilepsy or symptomatic partial epilepsy or related symptoms, relative to a patient who does not receive the therapy.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

“Chronic” administration refers to administration of the medicament(s) in a continuous as opposed to acute mode, so as to maintain the initial therapeutic effect (activity) for an extended period of time. “Intermittent” administration refers to treatment that is not administered consecutively without interruption, but rather is cyclic/periodic in nature.

As used herein, administration “conjointly” with another compound or composition includes simultaneous administration and/or administration at different times. Conjoint administration also encompasses administration as a co-formulation or administration as separate compositions, including at different dosing frequencies or intervals, and using the same route of administration or different routes of administration.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### Anti-Complement C1q Antibodies

The anti-C1q antibodies disclosed herein are potent inhibitors of C1q and can be dosed for continuous inhibition in both the periphery and CNS over any period, and then optionally withdrawn to allow for return of normal C1q function at times when its activity may be important for CNS repair. Results obtained with anti-C1q antibodies disclosed herein in animal studies can be readily carried forward into the clinic with a humanized version of the same antibody (disclosed antibodies herein cross react with mouse and human C1q), as well as with fragments and/or derivatives thereof.

C1q is a large multimeric protein of 460 kDa consisting of 18 polypeptide chains (6 C1q A chains, 6 C1q B chains, and 6 C1q C chains). C1r and C1s complement proteins bind to the C1q tail region to form the C1 complex (C1qr₂s₂).

Suitable inhibitors include an antibody that binds complement factor C1q and/or C1q in the C1 complex of the classical complement activation pathway. The bound complement factor may be derived, without limitation, from any organism having a complement system, including any mammalian organism such as human, mouse, rat, rabbit, monkey, dog, cat, cow, horse, camel, sheep, goat, or pig.

As used herein “C1 complex” refers to a protein complex that may include, without limitation, one C1q protein, two C1r proteins, and two C1s proteins (e.g., C1qr²s²).

As used herein “complement factor C1q” refers to both wild type sequences and naturally occurring variant sequences.

A non-limiting example of a complement factor C1q recognized by antibodies of this disclosure is human C1q, including the three polypeptide chains A, B, and C:

C1q, chain A (homo sapiens),
Accession No. Protein
Data Base: NP_057075.1; GenBank No.: NM_015991:
>gi|7705753|ref|NP_057075.1|complement C1q
subcomponent subunit A precursor
[Homo sapiens]
(SEQ ID NO: 1)
MEGPRGWLVLCVLAISLASMVTEDLCRAPDGKKGEAGRPG
RRGRPGLKGEQGEPGAPGIRTGIQGLKGDQGEPGPSGNPG
KVGYPGPSGPLGARGIPGIKGTKGSPGNIKDQPRPAFSAI
RRNPPMGGNVVIFDTVITNQEEPYQNHSGRFVCTVPGYYY
FTFQVLSQWEICLSIVSSSRGQVRRSLGFCDTTNKGLFQV
VSGGMVLQLQQGDQVWVEKDPKKGHIYQGSEADSVFSGFL
IFPSA.
C1q, chain B (homo sapiens),
Accession No. Protein
Data Base: NP_000482.3;
GenBank No.: NM_000491.3:
>gi|87298828|ref|NP_000482.3|complement C1q
subcomponent subunit B precursor
[Homo sapiens]
(SEQ ID NO: 2)
MMMKIPWGSIPVLMLLLLLGLIDISQAQLSCTGPPAIPGI
PGIPGTPGPDGQPGTPGIKGEKGLPGLAGDHGEFGEKGDP
GIPGNPGKVGPKGPMGPKGGPGAPGAPGPKGESGDYKATQ
KIAFSATRTINVPLRRDQTIRFDHVITNMNNNYEPRSGKF
TCKVPGLYYFTYHASSRGNLCVNLMRGRERAQKVVTFCDY
AYNTFQVTTGGMVLKLEQGENVFLQATDKNSLLGMEGANS
IFSGFLLFPDMEA.
C1q, chain C (homo sapiens),
Accession No. Protein
Data Base: NP_001107573.1;
GenBank No.: NM 001114101.1:
>gi|166235903|ref|NP_001107573.1|complement
C1q
subcomponent subunit C precursor
[Homo sapiens]
(SEQ ID NO: 3)
MDVGPSSLPHLGLKLLLLLLLLPLRGQANTGCYGIPGMPG
LPGAPGKDGYDGLPGPKGEPGIPAIPGIRGPKGQKGEPGL
PGHPGKNGPMGPPGMPGVPGPMGIPGEPGEEGRYKQKFQS
VFTVTRQTHQPPAPNSLIRFNAVLTNPQGDYDTSTGKFTC
KVPGLYYFVYHASHTANLCVLLYRSGVKVVTFCGHTSKTN
QVNSGGVLLRLQVGEEVWLAVNDYYDMVGIQGSDSVFSGF
LLFPD.

Accordingly, an anti-C1q antibody of the present disclosure may bind to polypeptide chain A, polypeptide chain B, and/or polypeptide chain C of a C1q protein. In some embodiments, an anti-C1q antibody of the present disclosure binds to polypeptide chain A, polypeptide chain B, and/or polypeptide chain C of human C1q or a homolog thereof, such as mouse, rat, rabbit, monkey, dog, cat, cow, horse, camel, sheep, goat, or pig C1q. In some embodiments, the anti-C1q antibody is a human antibody, a humanized antibody, or a chimeric antibody.

Suitable antibodies include an antibody that binds complement C1q protein (i.e., an anti-complement C1q antibody, also referred to herein as an anti-C1q antibody and a C1q antibody) and a nucleic acid molecule that encodes such an antibody for a method of preventing, reducing risk of developing, or treating epilepsy, such as an idiopathic generalized epilepsy, idiopathic partial epilepsy, symptomatic generalized epilepsy or symptomatic partial epilepsy.

Other anti-C1q antibodies suitable for binding to C1q protein are well-known in the art and include, for example, antibodies Cat #: AF2379, AF1696, MAB1696, and MAB23791 (R&D System), NBP1-87492, NB100-64420, H00000712-BO1P, H00000712-D01P, and H00000712-D01 (Novus Biologicals), MA1-83963, MA1-40311, PA5-14208, PA5-29586, and PA1-36177 (ThermoFisher Scientific), ab71940, ab11861, ab4223, ab72355, ab182451, ab46191, ab227072, ab182940, ab216979, and ab235454 (abcam), etc. Moreover, multiple siRNA, shRNA, CRISPR constructs for reducing C1q expression can be found in the commercial product lists of the above-referenced companies, such as SiRNA product #sc-43651, sc-44962, sc-105153, sc-141842, ShRNA product #sc-43651-SH, sc-43651-V, sc-44962-SH, sc-44962-V, sc-105153-SH, sc-105153-V, sc-141842-SH, sc-141842-V, CRISPR product #sc-419385, sc-419385-HDR, sc-419385-NIC, sc-419385-NIC-2, sc-402156, sc-402156-KO-2, sc-404309, sc-404309-HDR, sc-404309-NIC, sc-404309-NIC-2, sc-419386, sc-419386-HDR, sc-419386-NIC, sc-419386-NIC-2 (Santa Cruz Biotechnology, etc).

All sequences mentioned in the following twenty paragraphs are incorporated by reference from U.S. Pat. No. 9,708,394, which is hereby incorporated by reference for the antibodies and related compositions that it discloses.

### Light Chain and Heavy Chain Variable Domain Sequences of Antibody M1

Using standard techniques, the nucleic acid and amino acid sequences encoding the light chain variable and the heavy chain variable domain of antibody M1 were determined. The amino acid sequence of the light chain variable domain of antibody M1 is:

(SEQ ID NO: 4)
DVQITQSPSYLAASPGETITINCRASKSINKYLAWYQEKPG
KTNKLLIYSGSTLQSGIPSRFSGSGSGTDFTLTISSLEPED
FAMYYCQQHNEYPLTFGAGTKLELK.

The hyper variable regions (HVRs) of the light chain variable domain are depicted in bolded and underlined text. In some embodiments, the HVR-L1 of the M1 light chain variable domain has the sequence RASKSINKYLA (SEQ ID NO:5), the HVR-L2 of the M1 light chain variable domain has the sequence SGSTLQS (SEQ ID NO:6), and the HVR-L3 of the M1 light chain variable domain has the sequence QQHNEYPLT (SEQ ID NO:7).

The amino acid sequence of the heavy chain variable domain of antibody M1 is:

(SEQ ID NO: 8)
QVQLQQPGAELVKPGASVKLSCKSSGYHFTSYWMHWVKQRPG
QGLEWIGVIHPNSGSINYNEKFESKATLTVDKSSSTAYMQLS
SLTSEDSAVYYCAGERDSTEVLPMDYWGQGTSVTVSS

The hyper variable regions (HVRs) of the heavy chain variable domain are depicted in bolded and underlined text. In some embodiments, the HVR-H1 of the M1 heavy chain variable domain has the sequence GYHFTSYWMH (SEQ ID NO:9), the HVR-H2 of the M1 heavy chain variable domain has the sequence VIHPNSGSINYNEKFES (SEQ ID NO:10), and the HVR-H3 of the M1 heavy chain variable domain has the sequence ERDSTEVLPMDY (SEQ ID NO:11).

The nucleic acid sequence encoding the light chain variable domain was determined to be:

(SEQ ID NO: 12)
GATGTCCAGATAACCCAGTCTCCATCTTATCTTGCTGCAT
CTCCTGGAGAAACCATTACTATTAATTGCAGGGCAAGTAA
GAGCATTAACAAATATTTAGCCTGGTATCAAGAGAAACCT
GGGAAAACTAATAAGCTTCTTATCTACTCTGGATCCACTT
TGCAATCTGGAATTCCATCAAGGTTCAGTGGCAGTGGATC
TGGTACAGATTTCACTCTCACCATCAGTAGCCTGGAGCCT
GAAGATTTTGCAATGTATTACTGTCAACAACATAATGAAT
ACCCGCTCACGTTCGGTGCTGGGACCAAGCTGGAGCTGAA
A.

The nucleic acid sequence encoding the heavy chain variable domain was determined to be:

(SEQ ID NO: 13)
CAGGTCCAACTGCAGCAGCCTGGGGCTGAGCTGGTAAAGC
CTGGGGCTTCAGTGAAGTTGTCCTGCAAGTCTTCTGGCTA
CCATTTCACCAGCTACTGGATGCACTGGGTGAAGCAGAGG
CCTGGACAAGGCCTTGAGTGGATTGGAGTGATTCATCCTA
ATAGTGGTAGTATTAACTACAATGAGAAGTTCGAGAGCAA
GGCCACACTGACTGTAGACAAATCCTCCAGCACAGCCTAC
ATGCAACTCAGCAGCCTGACATCTGAGGACTCGGCGGTCT
ATTATTGTGCAGGAGAGAGAGATTCTACGGAGGTTCTCCC
TATGGACTACTGGGGTCAAGGAACCTCAGTCACCGTCTCC
TCA

### Deposit of Material

The following materials have been deposited according to the Budapest Treaty in the American Type Culture Collection, ATCC Patent Depository, 10801 University Blvd., Manassas, Va. 20110-2209, USA (ATCC):

| | | Deposit | ATCC |
|---|---|---|---|
| Sample ID | Isotype | Date | Accession No. |
| Mouse hybridoma C1qM1 | IgG1, | Jun. 6, | PTA-120399 |
| 7788-1(M) 051613 producing | kappa | 2013 | |
| anti-C1q antibody M1 | | | |

The hybridoma cell line producing the M1 antibody (mouse hybridoma C1qM1 7788-1(M) 051613) has been deposited with ATCC under conditions that assure that access to the culture will be available during pendency of the patent application and for a period of 30 years, or 5 years after the most recent request, or for the effective life of the patent, whichever is longer. A deposit will be replaced if the deposit becomes nonviable during that period. The deposit is available as required by foreign patent laws in countries wherein counterparts of the subject application, or its progeny are filed. However, it should be understood that the availability of the deposit does not constitute a license to practice the subject invention in derogation of patent rights granted by governmental action.

Disclosed herein are methods of administering an anti-C1q antibody comprising a light chain variable domain and a heavy chain variable domain. The antibody may bind to at least human C1q, mouse C1q, or rat C1q. The antibody may be a humanized antibody, a chimeric antibody, or a human antibody. The antibody may be a monoclonal antibody, an antibody fragment thereof, and/or an antibody derivative thereof. The light chain variable domain comprises the HVR-L1, HVR-L2, and HVR-L3 of the monoclonal antibody M1 produced by a hybridoma cell line deposited with Accession Number PTA-120399. The heavy chain variable domain comprises the HVR-H1, HVR-H2, and HVR-H3 of the monoclonal antibody M1 produced by a hybridoma cell line deposited with ATCC Accession Number PTA-120399.

In some embodiments, the amino acid sequence of the light chain variable domain and heavy chain variable domain comprise one or more of SEQ ID NO:5 of HVR-L1, SEQ ID NO:6 of HVR-L2, SEQ ID NO:7 of HVR-L3, SEQ ID NO:9 of HVR-H1, SEQ ID NO:10 of HVR-H2, and SEQ ID NO:11 of HVR-H3.

The antibody may comprise a light chain variable domain amino acid sequence that is at least 85%, 90%, or 95% identical to SEQ ID NO:4, preferably while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:8, preferably while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO:11).

Disclosed herein are methods of administering an anti-C1q antibody, which inhibits the interaction between C1q and an autoantibody. In preferred embodiments, the anti-C1q antibody causes clearance of C1q from the circulation or tissue.

The anti-C1q antibody may bind to a C1q protein, and binds to one or more amino acids of the C1q protein within amino acid residues selected from (a) amino acid residues 196-226 of SEQ ID NO:1 (SEQ ID NO:16), or amino acid residues of a C1q protein chain A (C1qA) corresponding to amino acid residues 196-226 (GLFQVVSGGMVLQLQQGDQVWVEKDPKKGHI) of SEQ ID NO:1 (SEQ ID NO:16); (b) amino acid residues 196-221 of SEQ ID NO:1 (SEQ ID NO:17), or amino acid residues of a C1qA corresponding to amino acid residues 196-221 (GLFQVVSGGMVLQLQQGDQVWVEKDP) of SEQ ID. NO:1 (SEQ ID NO:17); (c) amino acid residues 202-221 of SEQ ID NO:1 (SEQ ID NO:18), or amino acid residues of a C1qA corresponding to amino acid residues 202-221 (SGGMVLQLQQGDQVWVEKDP) of SEQ ID NO:1 (SEQ ID NO:18); (d) amino acid residues 202-219 of SEQ ID NO:1 (SEQ ID NO:19), or amino acid residues of a C1qA corresponding to amino acid residues 202-219 (SGGMVLQLQQGDQVWVEK) of SEQ ID NO:1 (SEQ ID NO:19); and (e) amino acid residues Lys 219 and/or Ser 202 of SEQ ID NO:1, or amino acid residues of a C1qA corresponding Lys 219 and/or Ser 202 of SEQ ID NO:1.

In some embodiments, the antibody further binds to one or more amino acids of the C1q protein within amino acid residues selected from: (a) amino acid residues 218-240 of SEQ ID NO:3 (SEQ ID NO:20) or amino acid residues of a C1q protein chain C (C1qC) corresponding to amino acid residues 218-240 (WLAVNDYYDMVGI QGSDSVFSGF) of SEQ ID NO:3 (SEQ ID NO:20); (b) amino acid residues 225-240 of SEQ ID NO:3 (SEQ ID NO:21) or amino acid residues of a C1qC corresponding to amino acid residues 225-240 (YDMVGI QGSDSVFSGF) of SEQ ID NO:3 (SEQ ID NO:21); (c) amino acid residues 225-232 of SEQ ID NO:3 (SEQ ID NO:22) or amino acid residues of a C1qC corresponding to amino acid residues 225-232 (YDMVGIQG) of SEQ ID NO:3 (SEQ ID NO:22); (d) amino acid residue Tyr 225 of SEQ ID NO:3 or an amino acid residue of a C1qC corresponding to amino acid residue Tyr 225 of SEQ ID NO:3; (e) amino acid residues 174-196 of SEQ ID NO:3 (SEQ ID NO:23) or amino acid residues of a C1qC corresponding to amino acid residues 174-196 (HTANLCVLLYRSGVKVVTFCGHT) of SEQ ID NO:3 (SEQ ID NO:23); (f) amino acid residues 184-192 of SEQ ID NO:3 (SEQ ID NO:24) or amino acid residues of a C1qC corresponding to amino acid residues 184-192 (RSGVKVVTF) of SEQ ID NO:3 (SEQ ID NO:24); (g) amino acid residues 185-187 of SEQ ID NO:3 or amino acid residues of a C1qC corresponding to amino acid residues 185-187 (SGV) of SEQ ID NO:3; (h) amino acid residue Ser 185 of SEQ ID NO:3 or an amino acid residue of a C1qC corresponding to amino acid residue Ser 185 of SEQ ID NO:3.

In certain embodiments, the anti-C1q antibody binds to amino acid residue Lys 219 and Ser 202 of the human C1qA as shown in SEQ ID NO:1 or amino acids of a human C1qA corresponding to Lys 219 and Ser 202 as shown in SEQ ID NO:1, and amino acid residue Tyr 225 of the human C1qC as shown in SEQ ID NO:3 or an amino acid residue of a human C1qC corresponding to Tyr 225 as shown in SEQ ID NO:3. In certain embodiments, the anti-C1q antibody binds to amino acid residue Lys 219 of the human C1qA as shown in SEQ ID NO:1 or an amino acid residue of a human C1qA corresponding to Lys 219 as shown in SEQ ID NO:1, and amino acid residue Ser 185 of the human C1qC as shown in SEQ ID NO:3 or an amino acid residue of a human C1qC corresponding to Ser 185 as shown in SEQ ID NO:3.

In some embodiments, the anti-C1q antibody binds to a C1q protein and binds to one or more amino acids of the C1q protein within amino acid residues selected from: (a) amino acid residues 218-240 of SEQ ID NO:3 (SEQ ID NO:20) or amino acid residues of a C1qC corresponding to amino acid residues 218-240 (WLAVNDYYDMVGI QGSDSVFSGF) of SEQ ID NO:3 (SEQ ID NO:20); (b) amino acid residues 225-240 of SEQ ID NO:3 (SEQ ID NO:21) or amino acid residues of a C1qC corresponding to amino acid residues 225-240 (YDMVGI QGSDSVFSGF) of SEQ ID NO:3 (SEQ ID NO:21); (c) amino acid residues 225-232 of SEQ ID NO:3 (SEQ ID NO:22) or amino acid residues of a C1qC corresponding to amino acid residues 225-232 (YDMVGIQG) of SEQ ID NO:3 (SEQ ID NO:22); (d) amino acid residue Tyr 225 of SEQ ID NO:3 or an amino acid residue of a C1qC corresponding to amino acid residue Tyr 225 of SEQ ID NO:3; (e) amino acid residues 174-196 of SEQ ID NO:3 (SEQ ID NO:23) or amino acid residues of a C1qC corresponding to amino acid residues 174-196 (HTANLCVLLYRSGVKVVTFCGHT) of SEQ ID NO:3 (SEQ ID NO:23); (f) amino acid residues 184-192 of SEQ ID NO:3 (SEQ ID NO:24) or amino acid residues of a C1qC corresponding to amino acid residues 184-192 (RSGVKVVTF) of SEQ ID NO:3 (SEQ ID NO:24); (g) amino acid residues 185-187 of SEQ ID NO:3 or amino acid residues of a C1qC corresponding to amino acid residues 185-187 (SGV) of SEQ ID NO:3; (h) amino acid residue Ser 185 of SEQ ID NO:3 or an amino acid residue of a C1qC corresponding to amino acid residue Ser 185 of SEQ ID NO:3.

In some embodiments, the anti-C1q antibody of this disclosure inhibits the interaction between C1q and C1s. In some embodiments, the anti-C1q antibody inhibits the interaction between C1q and C1r. In some embodiments, the anti-C1q antibody inhibits the interaction between C1q and C1s and between C1q and C1r. In some embodiments, the anti-C1q antibody inhibits the interaction between C1q and another antibody, such as an autoantibody. In preferred embodiments, the anti-C1q antibody causes clearance of C1q from the circulation or tissue. In some embodiments, the anti-C1q antibody inhibits the respective interactions, at a stoichiometry of less than 2.5:1; 2.0:1; 1.5:1; or 1.0:1. In some embodiments, the C1q antibody inhibits an interaction, such as the C1q-C1s interaction, at approximately equimolar concentrations of C1q and the anti-C1q antibody. In other embodiments, the anti-C1q antibody binds to C1q with a stoichiometry of less than 20:1; less than 19.5:1; less than 19:1; less than 18.5:1; less than 18:1; less than 17.5:1; less than 17:1; less than 16.5:1; less than 16:1; less than 15.5:1; less than 15:1; less than 14.5:1; less than 14:1; less than 13.5:1; less than 13:1; less than 12.5:1; less than 12:1; less than 11.5:1; less than 11:1; less than 10.5:1; less than 10:1; less than 9.5:1; less than 9:1; less than 8.5:1; less than 8:1; less than 7.5:1; less than 7:1; less than 6.5:1; less than 6:1; less than 5.5:1; less than 5:1; less than 4.5:1; less than 4:1; less than 3.5:1; less than 3:1; less than 2.5:1; less than 2.0:1; less than 1.5:1; or less than 1.0:1. In certain embodiments, the anti-C1q antibody binds C1q with a binding stoichiometry that ranges from 20:1 to 1.0:1 or less than 1.0:1. In certain embodiments, the anti-C1q antibody binds C1q with a binding stoichiometry that ranges from 6:1 to 1.0:1 or less than 1.0:1. In certain embodiments, the anti-C1q antibody binds C1q with a binding stoichiometry that ranges from 2.5:1 to 1.0:1 or less than 1.0:1. In some embodiments, the anti-C1q antibody inhibits the interaction between C1q and C1r, or between C1q and C1s, or between C1q and both C1r and C1s. In some embodiments, the anti-C1q antibody inhibits the interaction between C1q and C1r, between C1q and C1s, and/or between C1q and both C1r and C1s. In some embodiments, the anti-C1q antibody binds to the C1q A-chain. In other embodiments, the anti-C1q antibody binds to the C1q B-chain. In other embodiments, the anti-C1q antibody binds to the C1q C-chain. In some embodiments, the anti-C1q antibody binds to the C1q A-chain, the C1q B-chain and/or the C1q C-chain. In some embodiments, the anti-C1q antibody binds to the globular domain of the C1q A-chain, B-chain, and/or C-chain. In other embodiments, the anti-C1q antibody binds to the collagen-like domain of the C1q A-chain, the C1q B-chain, and/or the C1q C-chain.

Where antibodies of this disclosure inhibit the interaction between two or more complement factors, such as the interaction of C1q and C1s, or the interaction between C1q and C1r, the interaction occurring in the presence of the antibody may be reduced by at least 10%, at least 20%, at least 30%, at least 40%, at least 50%, at least 60%, at least 70%, at least 80%, at least 90%, at least 95%, or at least 99% relative to a control wherein the antibodies of this disclosure are absent. In certain embodiments, the interaction occurring in the presence of the antibody is reduced by an amount that ranges from at least 30% to at least 99% relative to a control wherein the antibodies of this disclosure are absent.

In some embodiments, the antibodies of this disclosure inhibit C2 or C4-cleavage by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. Methods for measuring C2 or C4-cleavage are well known in the art. The EC₅₀ values for antibodies of this disclosure with respect C2 or C4-cleavage may be less than 3 μg/ml; 2.5 μg/ml; 2.0 μg/ml; 1.5 μg/ml; 1.0 μg/ml; 0.5 μg/ml; 0.25 μg/ml; 0.1 μg/ml; 0.05 μg/ml. In some embodiments, the antibodies of this disclosure inhibit C2 or C4-cleavage at approximately equimolar concentrations of C1q and the respective anti-C1q antibody.

In some embodiments, the antibodies of this disclosure inhibit autoantibody-dependent and complement-dependent cytotoxicity (CDC) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. The EC₅₀ values for antibodies of this disclosure with respect to inhibition of autoantibody-dependent and complement-dependent cytotoxicity may be less than 3 μg/ml; 2.5 μg/ml; 2.0 μg/ml; 1.5 μg/ml; 1.0 μg/ml; 0.5 μg/ml; 0.25 μg/ml; 0.1 μg/ml; 0.05 μg/ml.

In some embodiments, the antibodies of this disclosure inhibit complement-dependent cell-mediated cytotoxicity (CDCC) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. Methods for measuring CDCC are well known in the art. The EC₅₀ values for antibodies of this disclosure with respect CDCC inhibition may be 1 less than 3 μg/ml; 2.5 μg/ml; 2.0 μg/ml; 1.5 μg/ml; 1.0 μg/ml; 0.5 μg/ml; 0.25 μg/ml; 0.1 μg/ml; 0.05 μg/ml. In some embodiments, the antibodies of this disclosure inhibit CDCC but not antibody-dependent cellular cytotoxicity (ADCC).

### Humanized Anti-Complement C1q Antibodies

Humanized antibodies of the present disclosure specifically bind to a complement factor C1q and/or C1q protein in the C1 complex of the classical complement pathway. The humanized anti-C1q antibody may specifically bind to human C1q, human and mouse C1q, to rat C1q, or human C1q, mouse C1q, and rat C1q.

All sequences mentioned in the following sixteen paragraphs are incorporated by reference from U.S. Pat. No. 10,316,081, which is hereby incorporated by reference for the antibodies and related compositions that it discloses.

In some embodiments, the human heavy chain constant region is a human IgG4 heavy chain constant region comprising the amino acid sequence of SEQ ID NO:47, or with at least 70%, at least about 75%, at least about 80%, at least about 85%, at least about 90% homology to SEQ ID NO: 47. The human IgG4 heavy chain constant region may comprise an Fc region with one or more modifications and/or amino acid substitutions according to Kabat numbering. In such cases, the Fc region comprises a leucine to glutamate amino acid substitution at position 248, wherein such a substitution inhibits the Fc region from interacting with an Fc receptor. In some embodiments, the Fc region comprises a serine to proline amino acid substitution at position 241, wherein such a substitution prevents arm switching in the antibody.

The amino acid sequence of human IgG4 (S241P L248E) heavy chain constant domain is:

(SEQ ID NO: 47)
ASTKGPSVFPLAPCSRSTSESTAALGCLVKDYFPEPVTVSW
NSGALTSGVHTFPAVLQSSGLYSLSSVVTVPSSSLGTKTY
TCNVDHKPSNTKVDKRVESKYGPPCPPCPAPEFEGGPSVF
LFPPKPKDTLMISRTPEVTCVVVDVSQEDPEVQFNWYVDG
VEVHNAKTKPREEQFNSTYRVVSVLTVLHQDWLNGKEYKC
KVSNKGLPSSIEKTISKAKGQPREPQVYTLPPSQEEMTKN
QVSLTCLVKGFYPSDIAVEWESNGQPENNYKTTPPVLDSD
GSFFLYSRLTVDKSRWQEGNVFSCSVMHEALHNHYTQKSL
SLSLGK.

The antibody may comprise a heavy chain variable domain and a light chain variable domain, wherein the heavy chain variable domain comprises an amino acid sequence selected from any one of SEQ ID NOs: 31-34, or an amino acid sequence with at least about 90% homology to the amino acid sequence selected from any one of SEQ ID NOs: 31-34. In certain such embodiments, the light chain variable domain comprises an amino acid sequence selected from any one of SEQ ID NOs: 35-38, or an amino acid sequence with at least about 90% homology to the amino acid sequence selected from any one of SEQ ID NOs: 35-38.

The amino acid sequence of heavy chain variable domain variant 1 (VH1) is:

(SEQ ID NO: 31)
QVQLVQSGAELKKPGASVKVSCKSSGYHFTSYWMHWVKQA
PGQGLEWIGVIHPNSGSINYNEKFESKATITVDKSTSTAY
MQLSSLTSEDSAVYYCAGERDSTEVLPMDYWGQGTSVTVS
S.
The hyper variable regions (HVRs) of VH1 are
depicted in bolded and underlined text.

The amino acid sequence of heavy chain variable domain variant 2 (VH2) is:

(SEQ ID NO: 32)
QVQLVQSGAELKKPGASVKVSCKSSGYHFTSYWMHWVKQAPG
QGLEWIGVIHPNSGSINYNEKFESRATITVDKSTSTAYMELS
SLRSEDTAVYYCAGERDSTEVLPMDYWGQGTTVTVSS.
The hyper variable regions (HVRs) of VH2 are
depicted in bolded and underlined text.

The amino acid sequence of heavy chain variable domain variant 3 (VH3) is:

(SEQ ID NO: 33)
QVQLVQSGAELKKPGASVKVSCKSSGYHFTSYWMHWVKQAPGQ
GLEWIGVIHPNSGSINYNEKFESRVTITVDKSTSTAYMELSSL
RSEDTAVYYCAGERDSTEVLPMDYWGQGTTVTVSS.
The hyper variable regions (HVRs) of VH3 are
depicted in bolded and underlined text.

The amino acid sequence of heavy chain variable domain variant 4 (VH4) is:

(SEQ ID NO: 34)
QVQLVQSGAELKKPGASVKVSCKSSGYHFTSYWMHWVRQAPGQG
LEWIGVIHPNSGSINYNEKFESRVTITVDKSTSTAYMELSSLRS
EDTAVYYCAGERDSTEVLPMDYWGQGTTVTVSS.
The hyper variable regions (HVRs) of VH4 are
depicted in bolded and underlined text.

The amino acid sequence of kappa light chain variable domain variant 1 (Vκ1) is:

(SEQ ID NO: 35)
DVQITQSPSYLAASLGERATINCRASKSINKYLAWYQQKPGKT
NKLLIYSGSTLQSGIPARFSGSGSGTDFTLTISSLEPEDFAMY
YCQQHNEYPLTFGQGTKLEIK.
The hyper variable regions (HVRs) of Vκ1
are depicted in bolded and underlined text.

The amino acid sequence of kappa light chain variable domain variant 2 (Vκ2) is:

(SEQ ID NO: 36)
DVQITQSPSSLSASLGERATINCRASKSINKYLAWYQQKPGKA
NKLLIYSGSTLQSGIPARFSGSGSGTDFTLTISSLEPEDFAMY
YCQQHNEYPLTFGQGTKLEIK.
The hyper variable regions (HVRs) of Vκ2
are depicted in bolded and underlined text.

The amino acid sequence of kappa light chain variable domain variant 3 (Vκ3) is:

(SEQ ID NO: 37)
DVQITQSPSSLSASLGERATINCRASKSINKYLAWYQQKPGKA
PKLLIYSGSTLQSGIPARFSGSGSGTDFTLTISSLEPEDFAMY
YCQQHNEYPLTFGQGTKLEIK.
The hyper variable regions (HVRs) of Vκ3
are depicted in bolded and underlined text.

The amino acid sequence of kappa light chain variable domain variant 4 (Vκ4) is:

(SEQ ID NO: 38)
DIQLTQSPSSLSASLGERATINCRASKSINKYLAWYQQKPGKA
PKLLIYSGSTLQSGIPARFSGSGSGTDFTLTISSLEPEDFAMY
YCQQHNEYPLTFGQGTKLEIK.
The hyper variable regions (HVRs) of Vκ4 are
depicted in bolded and underlined text.

The antibody may comprise a light chain variable domain amino acid sequence that is at least 85%, 90%, or 95% identical to SEQ ID NO:35-38 while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:31-34 while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO:11).

In some embodiments, humanized anti-C1q antibodies of the present disclosure include a heavy chain variable region that contains an Fab region and a heavy chain constant regions that contains an Fc region, where the Fab region specifically binds to a C1q protein of the present disclosure, but the Fc region is incapable of binding the C1q protein. In some embodiments, the Fc region is from a human IgG1, IgG2, IgG3, or IgG4 isotype. In some embodiments, the Fc region is incapable of inducing complement activity and/or incapable of inducing antibody-dependent cellular cytotoxicity (ADCC). In some embodiments, the Fc region comprises one or more modifications, including, without limitation, amino acid substitutions. In certain embodiments, the Fc region of humanized anti-C1q antibodies of the present disclosure comprise an amino acid substitution at position 248 according to Kabat numbering convention or a position corresponding to position 248 according to Kabat numbering convention, and/or at position 241 according to Kabat numbering convention or a position corresponding to position 241 according to Kabat numbering convention. In some embodiments, the amino acid substitution at position 248 or a positions corresponding to position 248 inhibits the Fc region from interacting with an Fc receptor. In some embodiments, the amino acid substitution at position 248 or a positions corresponding to position 248 is a leucine to glutamate amino acid substitution. In some embodiments, the amino acid substitution at position 241 or a positions corresponding to position 241 prevents arm switching in the antibody. In some embodiments, the amino acid substitution at position 241 or a positions corresponding to position 241 is a serine to proline amino acid substitution. In certain embodiments, the Fc region of humanized anti-C1q antibodies of the present disclosure comprises the amino acid sequence of SEQ ID NO: 47, or an amino acid sequence with at least about 70%, at least about 75%, at least about 80% at least about 85% at least about 90%, or at least about 95% homology to the amino acid sequence of SEQ ID NO: 47.

In some embodiments, humanized anti-C1q antibodies of the present disclosure may bind to a C1q protein and binds to one or more amino acids of the C1q protein within amino acid residues selected from (a) amino acid residues 196-226 of SEQ ID NO: 1 (SEQ ID NO:16), or amino acid residues of a C1q protein chain A (C1qA) corresponding to amino acid residues 196-226 (GLFQVVSGGMVLQLQQGDQVWVEKDPKKGHI) of SEQ ID NO: 1 (SEQ ID NO:16); (b) amino acid residues 196-221 of SEQ ID NO: 1 (SEQ ID NO:17), or amino acid residues of a C1qA corresponding to amino acid residues 196-221 (GLFQVVSGGMVLQLQQGDQVWVEKDP) of SEQ ID. NO: 1 (SEQ ID NO:17); (c) amino acid residues 202-221 of SEQ ID NO:1 (SEQ ID NO:18), or amino acid residues of a C1qA corresponding to amino acid residues 202-221 (SGGMVLQLQQGDQVWVEKDP) of SEQ ID NO: 1 (SEQ ID NO:18); (d) amino acid residues 202-219 of SEQ ID NO: 1 (SEQ ID NO:19), or amino acid residues of a C1qA corresponding to amino acid residues 202-219 (SGGMVLQLQQGDQVWVEK) of SEQ ID NO: 1 (SEQ ID NO:19); and (e) amino acid residues Lys 219 and/or Ser 202 of SEQ ID NO: 1, or amino acid residues of a C1qA corresponding Lys 219 and/or Ser 202 of SEQ ID NO: 1.

In some embodiments, the humanized anti-C1q antibodies may further binds to one or more amino acids of the C1q protein within amino acid residues selected from: (a) amino acid residues 218-240 of SEQ ID NO: 3 (SEQ ID NO:20) or amino acid residues of a C1q protein chain C (C1qC) corresponding to amino acid residues 218-240 (WLAVNDYYDMVGI QGSDSVFSGF) of SEQ ID NO: 3 (SEQ ID NO:20); (b) amino acid residues 225-240 of SEQ ID NO: 3 (SEQ ID NO:21) or amino acid residues of a C1qC corresponding to amino acid residues 225-240 (YDMVGI QGSDSVFSGF) of SEQ ID NO: 3 (SEQ ID NO:21); (c) amino acid residues 225-232 of SEQ ID NO: 3 (SEQ ID NO:22) or amino acid residues of a C1qC corresponding to amino acid residues 225-232 (YDMVGIQG) of SEQ ID NO: 3 (SEQ ID NO:22); (d) amino acid residue Tyr 225 of SEQ ID NO: 3 or an amino acid residue of a C1qC corresponding to amino acid residue Tyr 225 of SEQ ID NO: 3; (e) amino acid residues 174-196 of SEQ ID NO: 3 (SEQ ID NO:23) or amino acid residues of a C1qC corresponding to amino acid residues 174-196 (HTANLCVLLYRSGVKVVTFCGHT) of SEQ ID NO: 3 (SEQ ID NO:23); (f) amino acid residues 184-192 of SEQ ID NO: 3 (SEQ ID NO:24) or amino acid residues of a C1qC corresponding to amino acid residues 184-192 (RSGVKVVTF) of SEQ ID NO: 3 (SEQ ID NO:24); (g) amino acid residues 185-187 of SEQ ID NO: 3 or amino acid residues of a C1qC corresponding to amino acid residues 185-187 (SGV) of SEQ ID NO: 3; (h) amino acid residue Ser 185 of SEQ ID NO: 3 or an amino acid residue of a C1qC corresponding to amino acid residue Ser 185 of SEQ ID NO: 3.

In certain embodiments, humanized anti-C1q antibodies of the present disclosure may bind to amino acid residue Lys 219 and Ser 202 of the human C1qA as shown in SEQ ID NO: 1 or amino acids of a human C1qA corresponding to Lys 219 and Ser 202 as shown in SEQ ID NO: 1, and amino acid residue Tyr 225 of the human C1qC as shown in SEQ ID NO: 3 or an amino acid residue of a human C1qC corresponding to Tyr 225 as shown in SEQ ID NO: 3. In certain embodiments, the anti-C1q antibody binds to amino acid residue Lys 219 of the human C1qA as shown in SEQ ID NO: 1 or an amino acid residue of a human C1qA corresponding to Lys 219 as shown in SEQ ID NO: 1, and amino acid residue Ser 185 of the human C1qC as shown in SEQ ID NO: 3 or an amino acid residue of a human C1qC corresponding to Ser 185 as shown in SEQ ID NO: 3.

In some embodiments, humanized anti-C1q antibodies of the present disclosure may bind to a C1q protein and binds to one or more amino acids of the C1q protein within amino acid residues selected from: (a) amino acid residues 218-240 of SEQ ID NO: 3 (SEQ ID NO:20) or amino acid residues of a C1qC corresponding to amino acid residues 218-240 (WLAVNDYYDMVGI QGSDSVFSGF) of SEQ ID NO: 3 (SEQ ID NO:20); (b) amino acid residues 225-240 of SEQ ID NO: 3 (SEQ ID NO:21) or amino acid residues of a C1qC corresponding to amino acid residues 225-240 (YDMVGI QGSDSVFSGF) of SEQ ID NO: 3 (SEQ ID NO:21); (c) amino acid residues 225-232 of SEQ ID NO: 3 (SEQ ID NO:22) or amino acid residues of a C1qC corresponding to amino acid residues 225-232 (YDMVGIQG) of SEQ ID NO: 3 (SEQ ID NO:22); (d) amino acid residue Tyr 225 of SEQ ID NO: 3 or an amino acid residue of a C1qC corresponding to amino acid residue Tyr 225 of SEQ ID NO: 3; (e) amino acid residues 174-196 of SEQ ID NO: 3 (SEQ ID NO:23) or amino acid residues of a C1qC corresponding to amino acid residues 174-196 (HTANLCVLLYRSGVKVVTFCGHT) of SEQ ID NO: 3 (SEQ ID NO:23); (f) amino acid residues 184-192 of SEQ ID NO: 3 (SEQ ID NO:24) or amino acid residues of a C1qC corresponding to amino acid residues 184-192 (RSGVKVVTF) of SEQ ID NO: 3 (SEQ ID NO:24); (g) amino acid residues 185-187 of SEQ ID NO: 3 or amino acid residues of a C1qC corresponding to amino acid residues 185-187 (SGV) of SEQ ID NO: 3; (h) amino acid residue Ser 185 of SEQ ID NO: 3 or an amino acid residue of a C1qC corresponding to amino acid residue Ser 185 of SEQ ID NO: 3.

### Anti-C1q Fab Fragment

Before the advent of recombinant DNA technology, proteolytic enzymes (proteases) that cleave polypeptide sequences have been used to dissect the structure of antibody molecules and to determine which parts of the molecule are responsible for its various functions. Limited digestion with the protease papain cleaves antibody molecules into three fragments. Two fragments, known as Fab fragments, are identical and contain the antigen-binding activity. The Fab fragments correspond to the two identical arms of the antibody molecule, each of which consists of a complete light chain paired with the VH and C_{H}1 domains of a heavy chain. The other fragment contains no antigen binding activity but was originally observed to crystallize readily, and for this reason was named the Fc fragment (Fragment crystallizable). When Fab molecules were compared to IgG molecules, it was found that Fab are superior to IgG for certain in vivo applications due to their higher mobility and tissue penetration capability, their reduced circulatory half-life, their ability to bind antigen monovalently without mediating antibody effector functions, and their lower immunogenicity.

The Fab molecule is an artificial ˜50-kDa fragment of the Ig molecule with a heavy chain shortened by constant domains C_{H}2 and C_{H}3. Two heterophilic (V_{L}-V_{H} and C_{L}-C_{H}1) domain interactions underlie the two-chain structure of the Fab molecule, which is further stabilized by a disulfide bridge between C_{L} and C_{H}1. Fab and IgG have identical antigen binding sites formed by six complementarity-determining regions (CDRs), three each from V_{L} and V_{H} (LCDR1, LCDR2, LCDR3 and HCDR1, HCDR2, HCDR3). The CDRs define the hypervariable antigen binding site of antibodies. The highest sequence variation is found in LCDR3 and HCDR3, which in natural immune systems are generated by the rearrangement of V_{L} and J_{L} genes or V_{H}, D_{H} and J_{H} genes, respectively. LCDR3 and HCDR3 typically form the core of the antigen binding site. The conserved regions that connect and display the six CDRs are referred to as framework regions. In the three-dimensional structure of the variable domain, the framework regions form a sandwich of two opposing antiparallel pi-sheets that are linked by hypervariable CDR loops on the outside and by a conserved disulfide bridge on the inside. This unique combination of stability and versatility of the antigen binding site of Fab and IgG underlie its success in clinical practice for the diagnosis, monitoring, prevention, and treatment of disease.

All anti-C1q antibody Fab fragment sequences are incorporated by reference from U.S. patent application Ser. No. 15/360,549, which is hereby incorporated by reference for the antibodies and related compositions that it discloses.

In certain embodiments, the present disclosure provides an anti-C1q antibody Fab fragment that binds to a C1q protein comprising a heavy (V_{H}/C_{H}1) and light chain (V_{L}/C_{L}), wherein the anti-C1q antibody Fab fragment has six complementarity determining regions (CDRs), three each from V_{L} and V_{H} (HCDR1, HCDR2, HCDR3, and LCDR1, LCDR2, LCDR3). The heavy chain of the antibody Fab fragment is truncated after the first heavy chain domain of IgG1 (SEQ ID NO: 39), and comprises the following amino acid sequence:

(SEQ ID NO: 39)
QVQLVQSGAELKKPGASVKVSCKSSGYHFTSYWMHWVKQA
PGQGLEWIGVIHPNSGSINYNEKFESRVTITVDKSTSTAY
MELSSLRSEDTAVYYCAGERDSTEVLPMDYWGQGTTVTVS
SASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDKTHT

The complementarity determining regions (CDRs) of SEQ ID NO:1 are depicted in bolded and underlined text.

The light chain domain of the antibody Fab fragment comprises the following amino acid sequence (SEQ ID NO: 40):

(SEQ ID NO: 40)
DVQITQSPSSLSASLGERATINCRASKSINKYLAWYQQKP
GKAPKLLIYSGSTLQSGIPARFSGSGSGTDFTLTISSLEP
EDFAMYYCQQHNEYPLTFGQGTKLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC

The complementarity determining regions (CDRs) of SEQ ID NO:2 are depicted in bolded and underlined text.

### Anti-Complement C1s Antibodies

Suitable inhibitors include an antibody that binds complement C1s protein (i.e., an anti-complement C1s antibody, also referred to herein as an anti-C1s antibody and a C1s antibody) and a nucleic acid molecule that encodes such an antibody. Complement C1s is an attractive target as it is upstream in the complement cascade and has a narrow range of substrate specificity. Furthermore it is possible to obtain antibodies (for example, but not limited to, monoclonal antibodies) that specifically bind the activated form of C1s.

All sequences mentioned in the following two paragraphs are incorporated by reference from U.S. patent application Ser. No. 14/890,811, which is hereby incorporated by reference for the antibodies and related compositions that it discloses.

In certain aspects, disclosed herein are methods of administering an anti-C1s antibody. The antibody may be a murine, humanized, or chimeric antibody. In some embodiments, the light chain variable domain comprises HVR-L1, HVR-L2, and HVR-L3, and the heavy chain comprises HVR-H1, HVR-H2, and HVR-H3 of a murine anti-human C1s monoclonal antibody 5A1 produced by a hybridoma cell line deposited with ATCC on May 15, 2013 or progeny thereof (ATCC Accession No. PTA-120351). In other embodiments, the light chain variable domain comprises the HVR-L1, HVR-L2, and HVR-L3 and the heavy chain variable domain comprises the HVR-H1, HVR-H2, and HVR-H3 of a murine anti-human C1s monoclonal antibody 5C12 produced by a hybridoma cell line deposited with ATCC on May 15, 2013, or progeny thereof (ATCC Accession No. PTA-120352).

In some embodiments, antibodies specifically bind to and inhibit a biological activity of C1s or the C1s proenzyme, such as C1s binding to C1q, C1s binding to C1r, or C1s binding to C2 or C4. The biological activity may be a proteolytic enzyme activity of C1s, the conversion of the C1s proenzyme to an active protease, or proteolytic cleavage of C2 or C4. In certain embodiments, the biological activity is activation of the classical complement activation pathway, activation of antibody and complement dependent cytotoxicity, or C1F hemolysis.

All sequences in the following sixty-two paragraphs are incorporated by reference from Van Vlasselaer, U.S. Pat. No. 8,877,197, which is hereby incorporated by reference for the antibodies and related compositions that it discloses.

Disclosed herein are methods of administering a humanized monoclonal antibody that specifically binds an epitope within a region encompassing domains IV and V of complement component C1s. In some cases, the antibody inhibits binding of C1s to complement component 4 (C4) and/or does not inhibit protease activity of C1s. In some embodiments, the method comprises administering a humanized monoclonal antibody that binds complement component C1s in a C1 complex with high avidity.

Disclosed herein are methods of administering an anti-C1s antibody with one or more of the complementarity determining regions (CDRs) of an antibody light chain variable region comprising amino acid sequence SEQ ID NO:57 and/or one or more of the CDRs of an antibody heavy chain variable region comprising amino acid sequence SEQ ID NO:58. The anti-C1s antibody may bind a human or rat complement C1s protein. In some embodiments, an anti-C1s antibody inhibits cleavage of at least one substrate cleaved by complement C1s protein.

In certain embodiments, the antibody comprises: a) a complementarity determining region (CDR) having an amino acid sequence selected from SEQ ID NO:51, SEQ ID NO:52, SEQ ID NO:53, SEQ ID NO:54, SEQ ID NO:55, and SEQ ID NO:56; and/or b) a CDR having an amino acid sequence selected from SEQ ID NO:62, SEQ ID NO:63, SEQ ID NO:53, SEQ ID NO:64, SEQ ID NO:65: and SEQ ID NO:66.

The antibody may comprise a CDR-L1 having amino acid sequence SEQ ID NO:51, a CDR-L2 having amino acid sequence SEQ ID NO:52, a CDR-L3 having amino acid sequence SEQ ID NO:53, a CDR-H1 having amino acid sequence SEQ ID NO:54, a CDR-H2 having amino acid sequence SEQ ID NO:55, and a CDR-H3 having amino acid sequence SEQ ID NO:56.

In other embodiments, the antibody may comprise light chain CDRs of a variable region with an amino acid sequence of SEQ ID NO:67, and/or heavy chain CDRs of a variable region with an amino acid sequence of SEQ ID NO:68.

The antibody can be a humanized antibody that specifically binds complement component C1s, wherein the antibody competes for binding the epitope with an antibody that comprises one or more of the CDRs of an antibody light chain variable region comprising amino acid sequence SEQ ID NO:57 or SEQ ID NO:67, and/or one or more of the CDRs of an antibody heavy chain variable region comprising amino acid sequence SEQ ID NO:58 or SEQ ID NO:68.

In other instances, the antibody can be a humanized antibody that specifically binds complement C1s, wherein the antibody is selected from: a) a humanized antibody that specifically binds an epitope within the complement C1s protein, wherein the antibody competes for binding the epitope with an antibody that comprises a CDR having an amino acid sequence selected from SEQ ID NO:51, SEQ ID NO:52, SEQ ID NO:53, SEQ ID NO:54, SEQ ID NO:55, and SEQ ID NO:56; and b) a humanized antibody that specifically binds an epitope within the complement C1s protein, wherein the antibody competes for binding the epitope with an antibody that comprises a CDR having an amino acid sequence selected from SEQ ID NO:62, SEQ ID NO:63, SEQ ID NO:53, SEQ ID NO:64, SEQ ID NO:65, and SEQ ID NO:66. In some cases, the antibody competes for binding the epitope with an antibody that comprises heavy and light chain CDRs comprising: a) SEQ ID NO:51, SEQ ID NO:52, SEQ ID NO:53, SEQ ID NO:69, SEQ ID NO:55, and SEQ ID NO:56; or b) SEQ ID NO:62, SEQ ID NO:63, SEQ ID NO:53, SEQ ID NO:64, SEQ ID NO:65, and SEQ ID NO:66.

The antibody may comprise a light chain region and a heavy chain region that are present in separate polypeptides. The antibody may comprise an Fc region.

Disclosed herein is an anti-C1s antibody comprising a light chain variable region of an amino acid sequence that is 90% identical to amino acid sequence SEQ ID NO:57, and a heavy chain variable region comprising an amino acid sequence that is 90% identical to amino acid sequence SEQ ID NO:58.

The anti-C1s antibody may be selected from an antigen binding fragment, Ig monomer, a Fab fragment, a F(ab′)2 fragment, a Fd fragment, a scFv, a scAb, a dAb, a Fv, a single domain heavy chain antibody, a single domain light chain antibody, a mono-specific antibody, a bi-specific antibody, or a multi-specific antibody.

Disclosed herein are methods of administering an antibody that competes for binding the epitope bound by antibody IPN003 (also referred to herein as “IPN-M34” or “M34” or “TNT003”), e.g., an antibody comprising a variable domain of antibody IPN003, such as antibody IPN003.

In some embodiments, the method comprises administering an antibody that specifically binds an epitope within a complement C1s protein. In some embodiments, the isolated anti-C1s antibody binds an activated C1s protein. In some embodiments, the isolated anti-C1s antibody binds an inactive form of C1s. In other instances, the isolated anti-C1s antibody binds both an activated C1s protein and an inactive form of C1s.

In some embodiments, the method comprises administering a monoclonal antibody that inhibits cleavage of C4, where the isolated monoclonal antibody does not inhibit cleavage of C2. In some embodiments, the method comprises administering a monoclonal antibody that inhibits cleavage of C2, where the isolated monoclonal antibody does not inhibit cleavage of C4. In some cases, the isolated monoclonal antibody is humanized. In some cases, the antibody inhibits a component of the classical complement pathway. In some cases, the component of the classical complement pathway that is inhibited by the antibody is C1s. The present disclosure also provides methods of treating a complement-mediated disease or disorder, by administering to an individual in need thereof an isolated monoclonal antibody that inhibits cleavage of C4, or a pharmaceutical composition comprising the isolated monoclonal antibody, where the isolated monoclonal antibody does not inhibit cleavage of C2.

In some embodiments, the method comprises administering a monoclonal antibody that inhibits cleavage of C2 or C4 by C1s, i.e., inhibits C1s-mediated proteolytic cleavage of C2 or C4. In some cases, the monoclonal antibody is humanized. In some cases, the antibody inhibits cleavage of C2 or C4 by C1s by inhibiting binding of C2 or C4 to C1s; for example, in some cases, the antibody inhibits C1s-mediated cleavage of C2 or C4 by inhibiting binding of C2 or C4 to a C2 or C4 binding site of C1s. Thus, in some cases, the antibody functions as a competitive inhibitor. The present disclosure also provides methods of treating epilepsy, such as an idiopathic generalized epilepsy, idiopathic partial epilepsy, symptomatic generalized epilepsy or symptomatic partial epilepsy, by administering to an individual in need thereof an isolated monoclonal antibody that inhibits cleavage of C2 or C4 by C1s, i.e., inhibits C1s-mediated proteolytic cleavage of C2 or C4.

In some embodiments, the method comprises administering a monoclonal antibody that inhibits cleavage of C4 by C1s, where the antibody does not inhibit cleavage of complement component C2 by C1s; i.e., the antibody inhibits C1s-mediated cleavage of C4, but does not inhibit C1s-mediated cleavage of C2. In some cases, the monoclonal antibody is humanized. In some cases, the monoclonal antibody inhibits binding of C4 to C1s, but does not inhibit binding of C2 to C1s. In some embodiments, the method comprises treating a complement-mediated disease or disorder, by administering to an individual in need thereof an isolated monoclonal antibody that inhibits cleavage of C4 by C1s, where the antibody does not inhibit cleavage of complement component C2 by C1s; i.e., the antibody inhibits C1s-mediated cleavage of C4, but does not inhibit C1s-mediated cleavage of C2. In some embodiments of the method, the antibody is humanized.

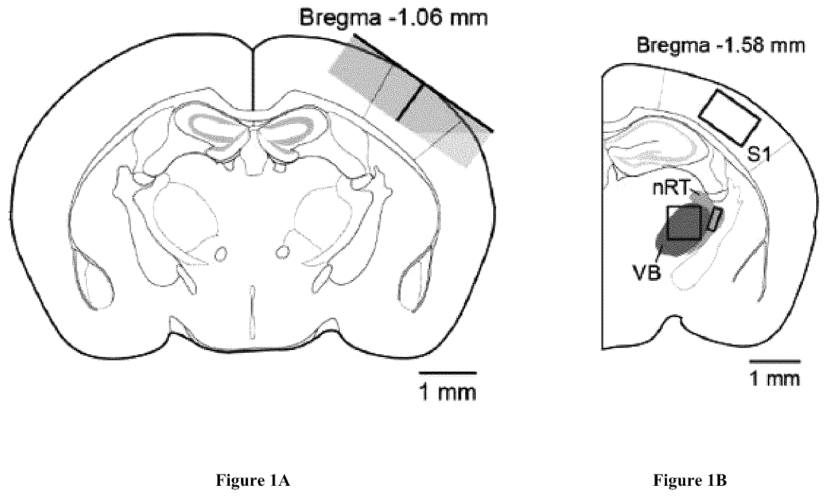

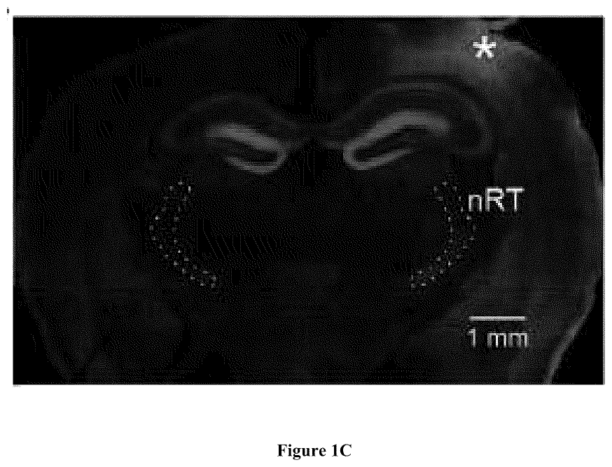

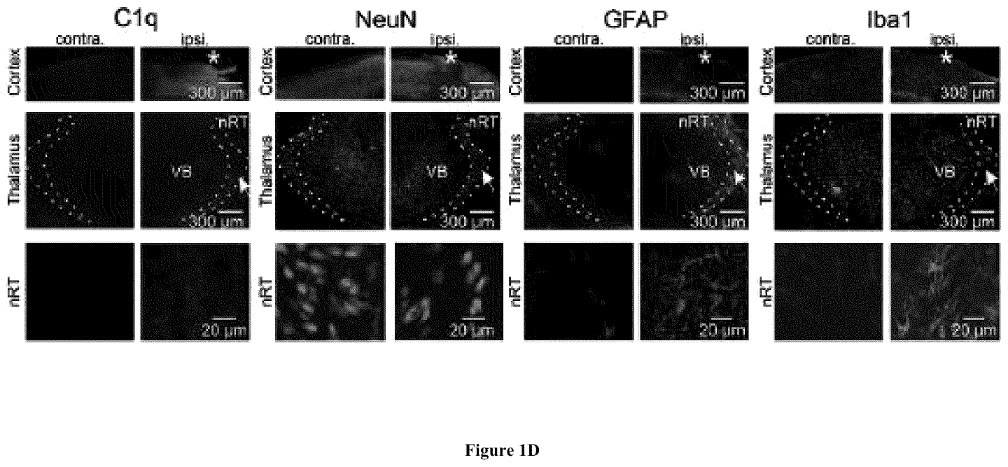

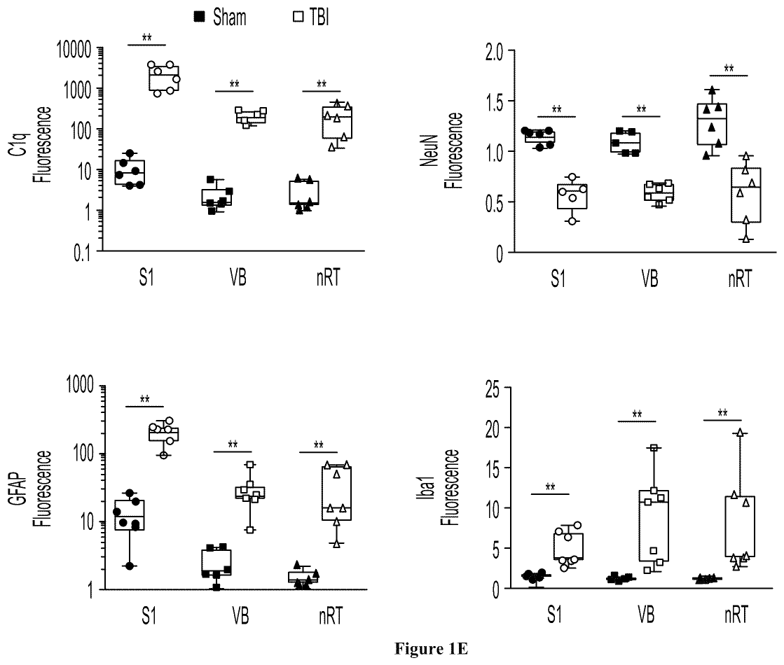

In some embodiments, the method comprises administering a humanized monoclonal antibody that specifically binds an epitope within a region encompassing domains IV and V of C1s. For example, the humanized monoclonal antibody specifically binds an epitope within amino acids 272-422 of the amino acid sequence depicted in FIG. 1 and set forth in SEQ ID NO:70. In some cases, the humanized monoclonal antibody specifically binds an epitope within amino acids 272-422 of the amino acid sequence depicted in FIG. 1 and set forth in SEQ ID NO:70, and inhibits binding of C4 to C1s. In some embodiments, the method comprises treating a complement-mediated disease or disorder, by administering to an individual in need thereof a humanized monoclonal antibody that specifically binds an epitope within amino acids 272-422 of the amino acid sequence depicted in FIG. 1 and set forth in SEQ ID NO:70, and inhibits binding of C4 to C1s.

In some embodiments, the method comprises administering a humanized monoclonal antibody that specifically binds a conformational epitope within a region encompassing domains IV and V of C1s. For example, the humanized monoclonal antibody that specifically binds a conformational epitope within amino acids 272-422 of the amino acid sequence depicted in FIG. 1 and set forth in SEQ ID NO:70. In some cases, the humanized monoclonal antibody specifically binds a conformational epitope within amino acids 272-422 of the amino acid sequence depicted in FIG. 1 and set forth in SEQ ID NO:70, and inhibits binding of C4 to C1s. In some embodiments, the method comprises epilepsy, such as an idiopathic generalized epilepsy, idiopathic partial epilepsy, symptomatic generalized epilepsy or symptomatic partial epilepsy, the method comprising administering to an individual in need thereof a humanized monoclonal antibody that specifically binds a conformational epitope within amino acids 272-422 of the amino acid sequence depicted in FIG. 1 and set forth in SEQ ID NO:70, and inhibits binding of C4 to C1s.

In some embodiments, the method comprises administering a monoclonal antibody that binds complement component C1s in a C1 complex. The C1 complex is composed of 6 molecules of C1q, 2 molecules of C1r, and 2 molecules of C1s. In some cases, the monoclonal antibody is humanized. Thus, in some cases, the humanized monoclonal antibody that binds complement component C1s in a C1 complex. In some cases, the antibody binds C1s present in a C1 complex with high avidity.

In some embodiments, the anti-C1s antibody (e.g., a subject antibody that specifically binds an epitope in a complement C1s protein) comprises: a) a light chain region comprising one, two, or three VL CDRs of an IPN003 antibody; and b) a heavy chain region comprising one, two, or three VH CDRs of an IPN003 antibody; where the VH and VL CDRs are as defined by Kabat (see, e.g., Table 1; and Kabat 1991).

In other embodiments, the anti-C1s antibody (e.g., a subject antibody that specifically binds an epitope in a complement C1s protein) comprises: a) a light chain region comprising one, two, or three VL CDRs of an IPN003 antibody; and b) a heavy chain region comprising one, two, or three VH CDRs of an IPN003 antibody; where the VH and VL CDRs are as defined by Chothia (see, e.g., Table 1, and Chothia 1987).

CDR amino acid sequences, and VL and VH amino acid sequences, of IPN003 antibody are provided in Table 2. Table 2 also provides the SEQ ID NOs assigned to each of the amino acid sequences.

In some embodiments, the anti-C1s antibody (e.g., a subject antibody that specifically binds an epitope in a complement C1s protein) comprises: a) a light chain region comprising one, two, or three CDRs selected from SEQ ID NO:51, SEQ ID NO:52, and SEQ ID NO:53; and b) a heavy chain region comprising one, two, or three CDRs selected from SEQ ID NO:54, SEQ ID NO:55, and SEQ ID NO:56. In some of these embodiments, the anti-C1s antibody includes a humanized VH and/or VL framework region.

SEQ ID NO. 51:
SSVSSSYLHWYQ;
SEQ ID NO. 52:
STSNLASGVP;
SEQ ID NO. 53:
HQYYRLPPIT;
SEQ ID NO. 54:
GFTFSNYAMSWV;
SEQ ID NO. 55:
ISSGGSHTYY;
SEQ ID NO. 56:
ARLFTGYAMDY.

In some embodiments, the anti-C1s antibody comprises a CDR having an amino acid sequence selected from SEQ ID NO:51, SEQ ID NO:52, SEQ ID NO:53, SEQ ID NO:54, SEQ ID NO:55, and SEQ ID NO:56.

In some embodiments, the anti-C1s antibody comprises a light chain variable region comprising amino acid sequences SEQ ID NO:51, SEQ ID NO:52, and SEQ ID NO:53.

In some embodiments, the anti-C1s antibody comprises a heavy chain variable region comprising amino acid sequences SEQ ID NO:54, SEQ ID NO:55, and SEQ ID NO:56.

In some embodiments, the anti-C1s antibody comprises a CDR-L1 having amino acid sequence SEQ ID NO:51, a CDR-L2 having amino acid sequence SEQ ID NO:52, a CDR-L3 having amino acid sequence SEQ ID NO:53, a CDR-H1 having amino acid sequence SEQ ID NO:54, a CDR-H2 having amino acid sequence SEQ ID NO:55, and a CDR-H3 having amino acid sequence SEQ ID NO:56.

In some embodiments, the anti-C1s antibody comprises a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:57.

SEQ ID NO. 57:
DIVMTQTTAIMSASLGERVTMTCTASSSVSSSYLHWYQQKPGSSP
KLWIYSTSNLASGVPARFSGSGSGTFYSLTISSMEAEDDATYYCH
QYYRLPPITFGAGTKLELK.

In some embodiments, the anti-C1s antibody comprises a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO. 58.

SEQ ID NO. 58:
QVKLEESGGALVKPGGSLKLSCAASGFTFSNYAMSWVRQIPEKRL
EWVATISSGGSHTYYLDSVKGRFTISRDNARDTLYLQMSSLRSED
TALYYCARLFTGYAMDYWGQGTSVT.

In some embodiments, the anti-C1s antibody comprises a light chain variable region comprising an amino acid sequence that is 90% identical to amino acid sequence SEQ ID NO:57.

In some embodiments, the anti-C1s antibody comprises a heavy chain variable region comprising an amino acid sequence that is 90% identical to amino acid sequence SEQ ID NO:58.

In some embodiments, the anti-C1s antibody comprises a light chain variable region comprising amino acid sequence SEQ ID NO:57.

In some embodiments, the anti-C1s antibody comprises a heavy chain variable region comprising amino acid sequence SEQ ID NO:58.

In some embodiments, the anti-C1s antibody comprises a light chain variable region comprising an amino acid sequence that is 90% identical to amino acid sequence SEQ ID NO:57 and a heavy chain variable region comprising an amino acid sequence that is 90% identical to amino acid sequence SEQ ID NO:58.

In some embodiments, the anti-C1s antibody comprises a light chain variable region comprising amino acid sequence SEQ ID NO:57 and a heavy chain variable region comprising amino acid sequence SEQ ID NO:58.

In some embodiments, the anti-C1s antibody specifically binds an epitope within the complement C1s protein, wherein the antibody competes for binding the epitope with an antibody that comprises light chain CDRs of an antibody light chain variable region comprising amino acid sequence SEQ ID NO:57 and heavy chain CDRs of an antibody heavy chain variable region comprising amino acid sequence SEQ ID NO:58.

In some embodiments, the anti-C1s antibody comprises light chain CDRs of an antibody light chain variable region comprising amino acid sequence SEQ ID NO:57 and heavy chain CDRs of an antibody heavy chain variable region comprising amino acid sequence SEQ ID NO:58.

In some embodiments, the anti-C1s antibody (e.g., a subject antibody that specifically binds an epitope in a complement C1s protein) comprises: a) a light chain region comprising one, two, or three CDRs selected from SEQ ID NO:62, SEQ ID NO:63, and SEQ ID NO:53; and b) a heavy chain region comprising one, two, or three CDRs selected from SEQ ID NO:64, SEQ ID NO:65, and SEQ ID NO:66.

SEQ ID NO. 62:
TASSSVSSSYLH;
SEQ ID NO. 63:
STSNLAS;
SEQ ID NO. 53:
HQYYRLPPIT;
SEQ ID NO. 64:
NYAMS;
SEQ ID NO. 65:
TISSGGSHTYYLDSVKG;
SEQ ID NO. 66:
LFTGYAMDY

In some embodiments, the anti-C1s antibody comprises a CDR having an amino acid sequence selected from SEQ ID NO:62, SEQ ID NO:63, SEQ ID NO:53, SEQ ID NO:64, SEQ ID NO:65, and SEQ ID NO:66.

In some embodiments, the anti-C1s antibody comprises a light chain variable region comprising amino acid sequences SEQ ID NO:62, SEQ ID NO:63, and SEQ ID NO:53.

In some embodiments, the anti-C1s antibody comprises a heavy chain variable region comprising amino acid sequences SEQ ID NO:64, SEQ ID NO:65, and SEQ ID NO:66.

In some embodiments, the anti-C1s antibody comprises a CDR-L1 having amino acid sequence SEQ ID NO:62, a CDR-L2 having amino acid sequence SEQ ID NO:63, a CDR-L3 having amino acid sequence SEQ ID NO:53, a CDR-H1 having amino acid sequence SEQ ID NO:64, a CDR-H2 having amino acid sequence SEQ ID NO:65, and a CDR-H3 having amino acid sequence SEQ ID NO:66.

In some embodiments, the anti-C1s antibody comprises a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:67.

SEQ ID NO. 67:
QIVLTQSPAIMSASLGERVTMTCTASSSVSSSYLHWYQQKP
GSSPKLWIYSTSNLASGVPARFSGSGSGTFYSLTISSMEAE
DDATYYCHQYYRLPPITFGAGTKLELK.

In some embodiments, the anti-C1s antibody comprises a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:68.

SEQ ID NO. 68:
EVMLVESGGALVKPGGSLKLSCAASGFTFSNYAMSWVRQIPE
KRLEWVATISSGGSHTYYLDSVKGRFTISRDNARDTLYLQMS
SLRSEDTALYYCARLFTGYAMDYWGQGTSVTVSS.

In some embodiments, the anti-C1s antibody comprises a light chain variable region comprising an amino acid sequence that is 90% identical to amino acid sequence SEQ ID NO:67.

In some embodiments, the anti-C1s antibody comprises a heavy chain variable region comprising an amino acid sequence that is 90% identical to amino acid sequence SEQ ID NO:68.

In some embodiments, the anti-C1s antibody comprises a light chain variable region comprising amino acid sequence SEQ ID NO:67.

In some embodiments, the anti-C1s antibody comprises a heavy chain variable region comprising amino acid sequence SEQ ID NO:68.

In some embodiments, the anti-C1s antibody comprises a light chain variable region comprising an amino acid sequence that is 90% identical to amino acid sequence SEQ ID NO:67 and a heavy chain variable region comprising an amino acid sequence that is 90% identical to amino acid sequence SEQ ID NO:68.

In some embodiments, the anti-C1s antibody comprises a light chain variable region comprising an amino acid sequence that is 95% identical to amino acid sequence SEQ ID NO:67 and a heavy chain variable region comprising an amino acid sequence that is 95% identical to amino acid sequence SEQ ID NO:68.

In some embodiments, the anti-C1s antibody comprises a light chain variable region comprising amino acid sequence SEQ ID NO:67 and a heavy chain variable region comprising amino acid sequence SEQ ID NO:68.

In some embodiments, the anti-C1s antibody specifically binds an epitope within the complement C1s protein, wherein the antibody competes for binding the epitope with an antibody that comprises light chain CDRs of an antibody light chain variable region comprising amino acid sequence SEQ ID NO:67 and heavy chain CDRs of an antibody heavy chain variable region comprising amino acid sequence SEQ ID NO:68.

In some embodiments, the anti-C1s antibody comprises light chain CDRs of an antibody light chain variable region comprising amino acid sequence SEQ ID NO:67 and heavy chain CDRs of an antibody heavy chain variable region comprising amino acid sequence SEQ ID NO:68.

In some embodiments, the anti-C1s antibody comprises a light chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:67.

In some embodiments, the anti-C1s antibody comprises a heavy chain variable region comprising an amino acid sequence that is 85%, 86%, 87%, 88%, 89%, 90%, 91%, 92%, 93%, 94%, 95%, 96%, 97%, 98% or 99% identical to the amino acid sequence set forth in SEQ ID NO:68.

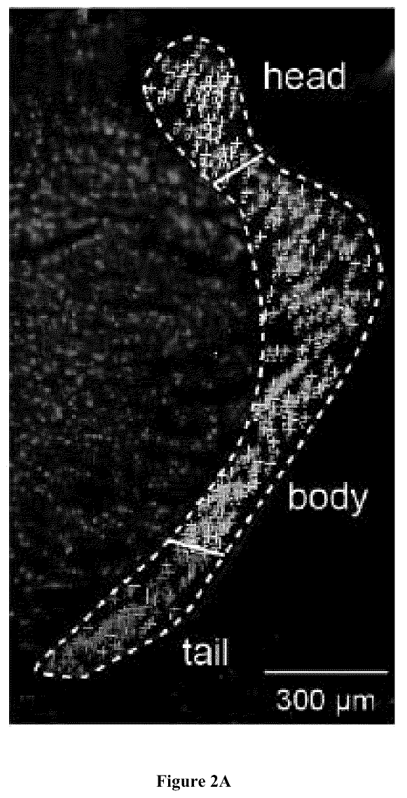

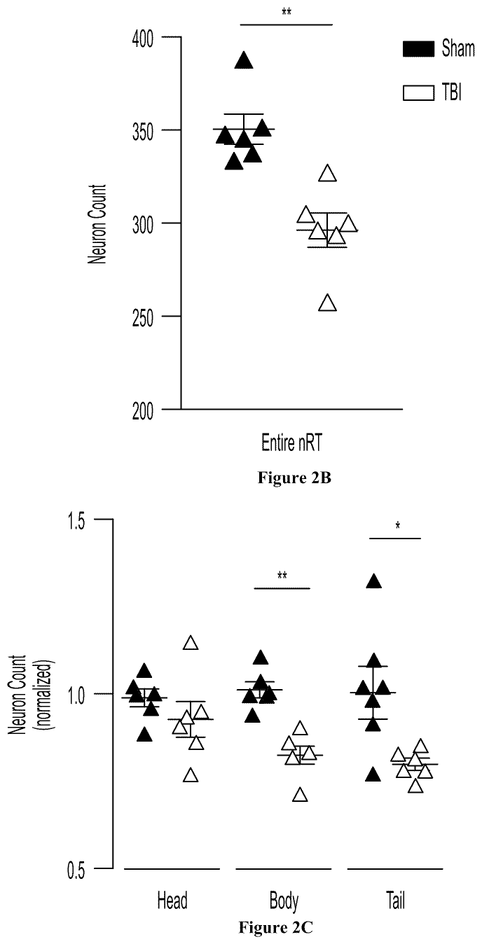

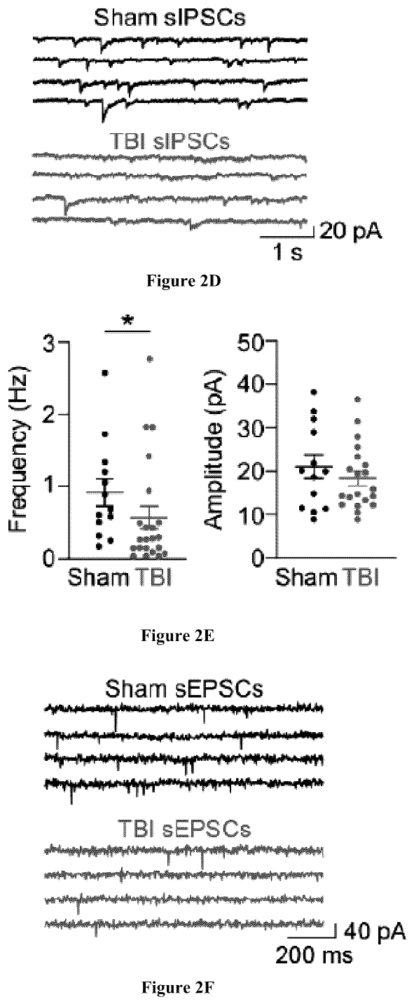

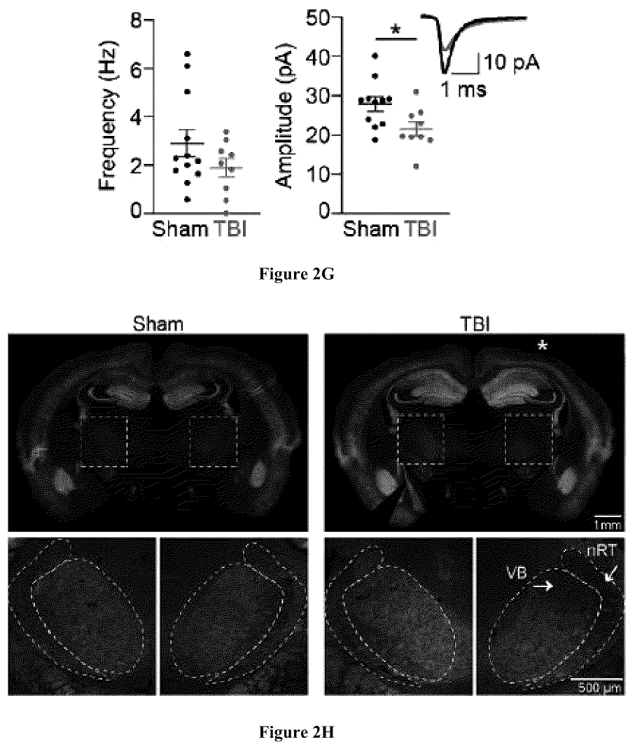

An anti-C1s antibody can comprise a heavy chain variable region comprising an amino acid sequence that is 85%, 86%, 87%, 88%, 89%, 90%, 91%, 92%, 93%, 94%, 95%, 96%, 97%, 98%, 99%, or 100% identical to the amino acid sequence set forth in SEQ ID NO:79 and depicted in FIG. 2 (VH variant 1).

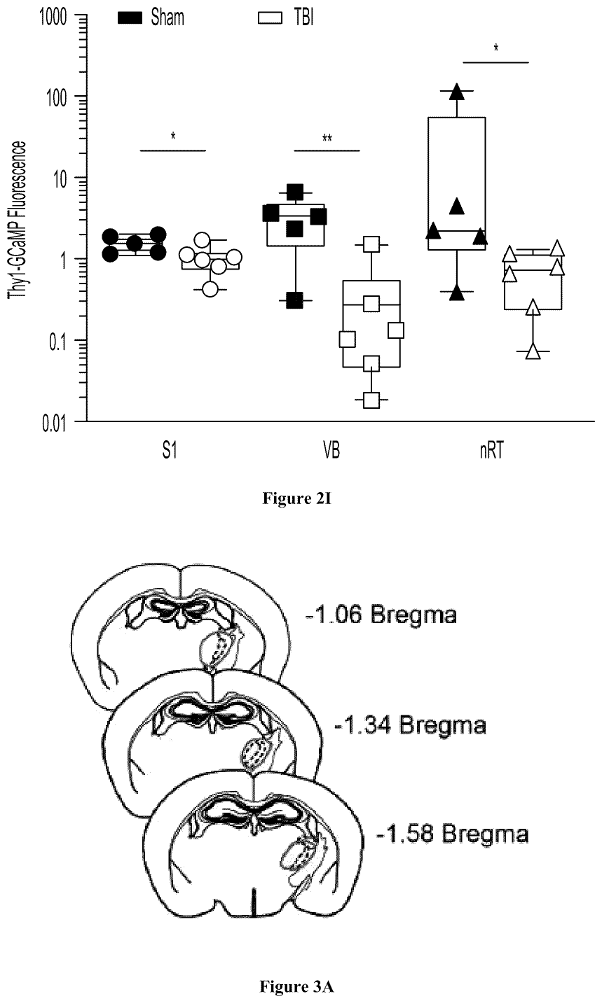

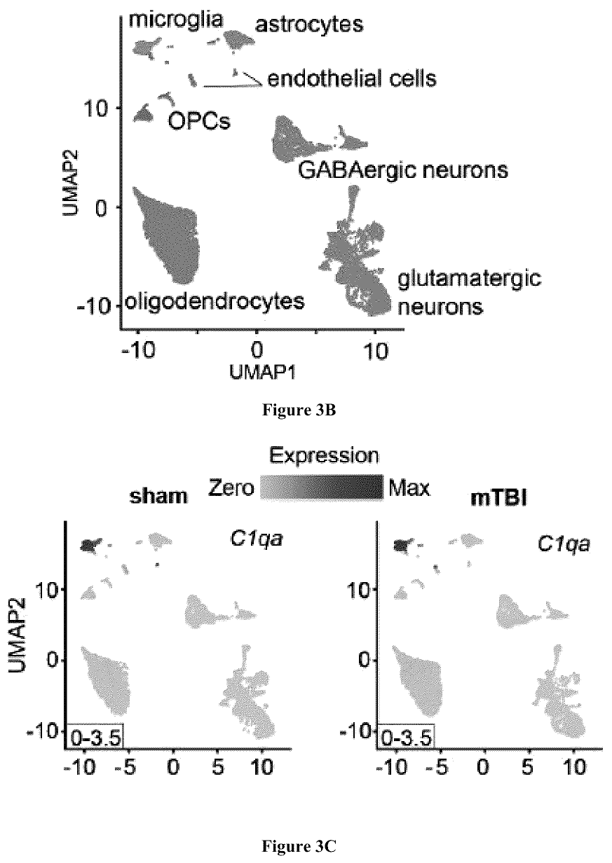

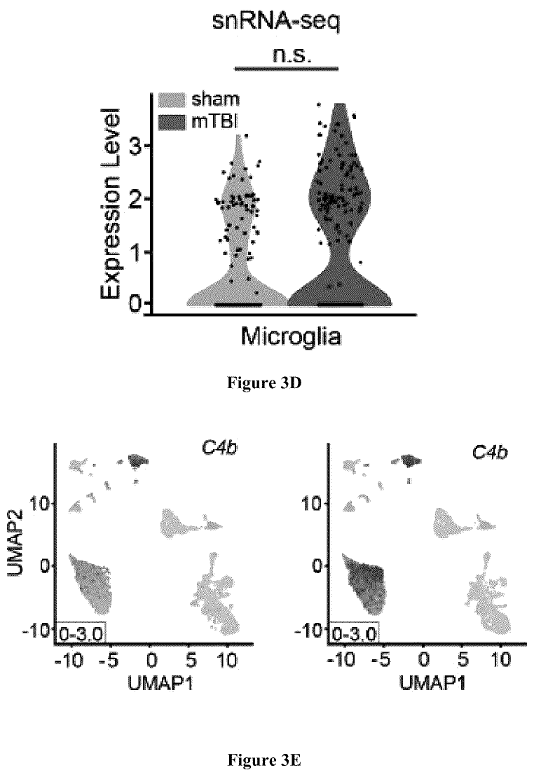

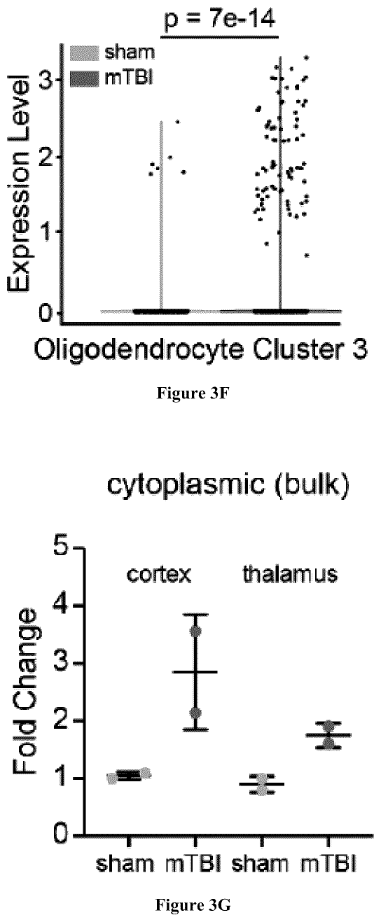

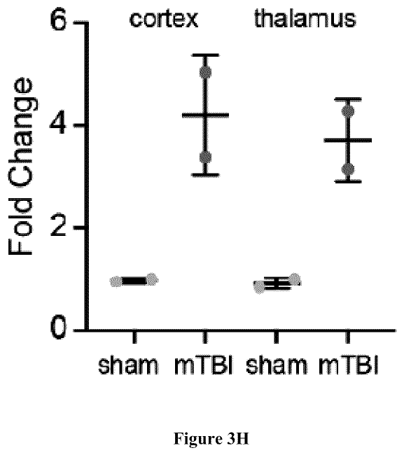

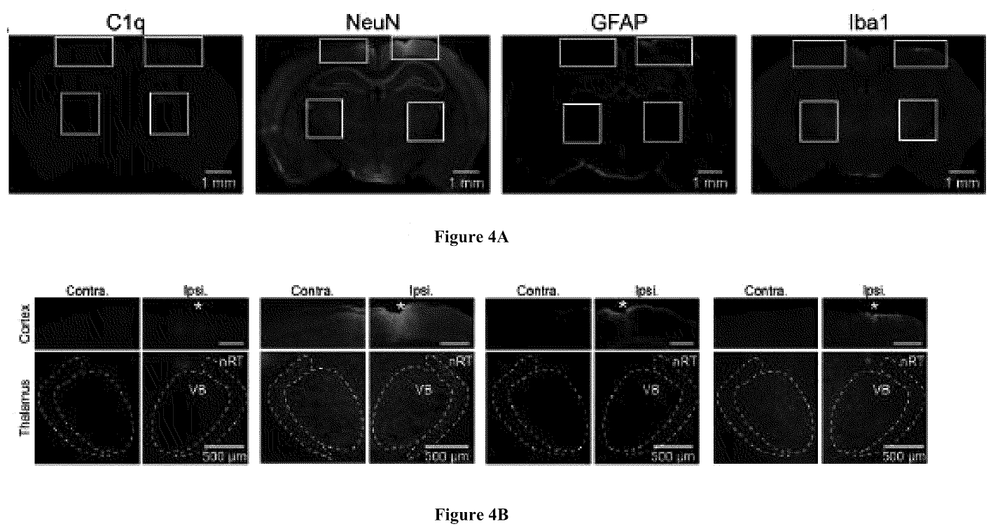

An anti-C1s antibody can comprise a heavy chain variable region comprising an amino acid sequence that is 85%, 86%, 87%, 88%, 89%, 90%, 91%, 92%, 93%, 94%, 95%, 96%, 97%, 98%, 99%, or 100% identical to the amino acid sequence set forth in SEQ ID NO:80 and depicted in FIG. 3 (VH variant 2).

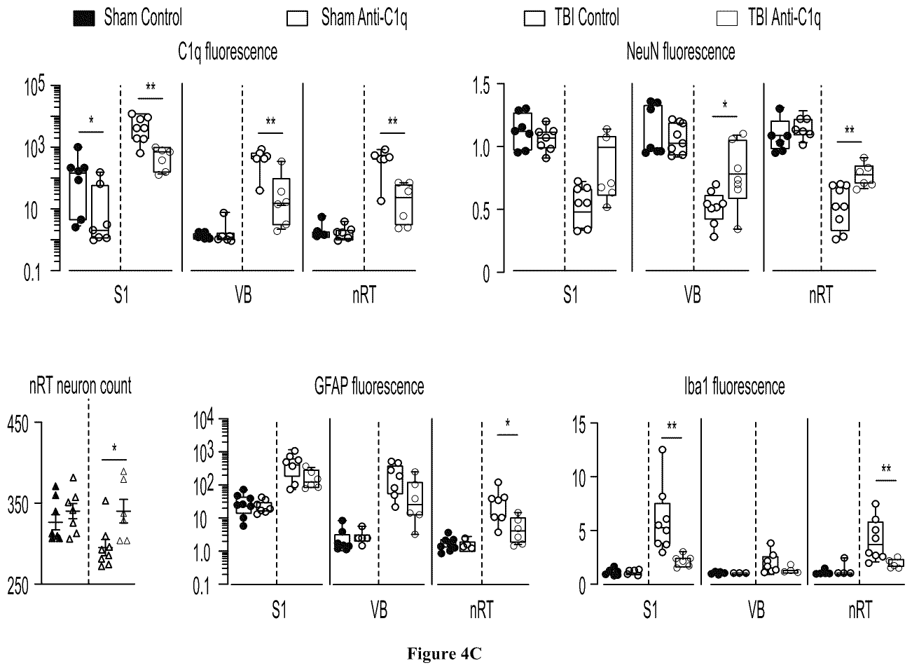

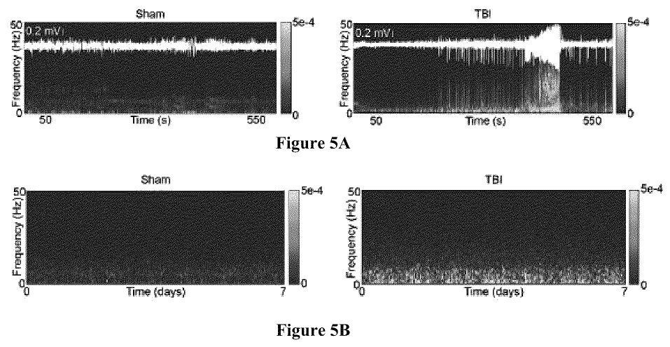

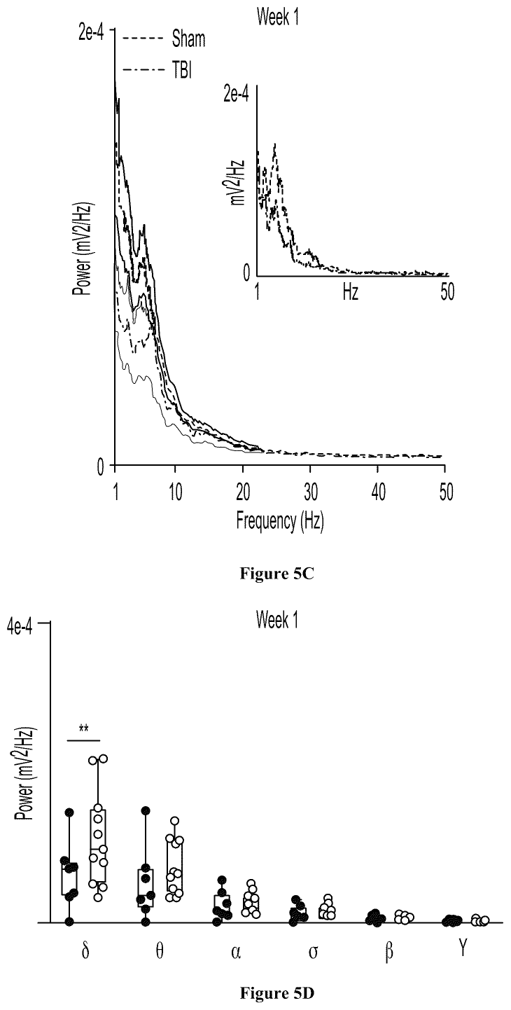

An anti-C1s antibody can comprise a heavy chain variable region comprising an amino acid sequence that is 85%, 86%, 87%, 88%, 89%, 90%, 91%, 92%, 93%, 94%, 95%, 96%, 97%, 98%, 99%, or 100% identical to the amino acid sequence set forth in SEQ ID NO:81 and depicted in FIG. 4 (VH variant 3).

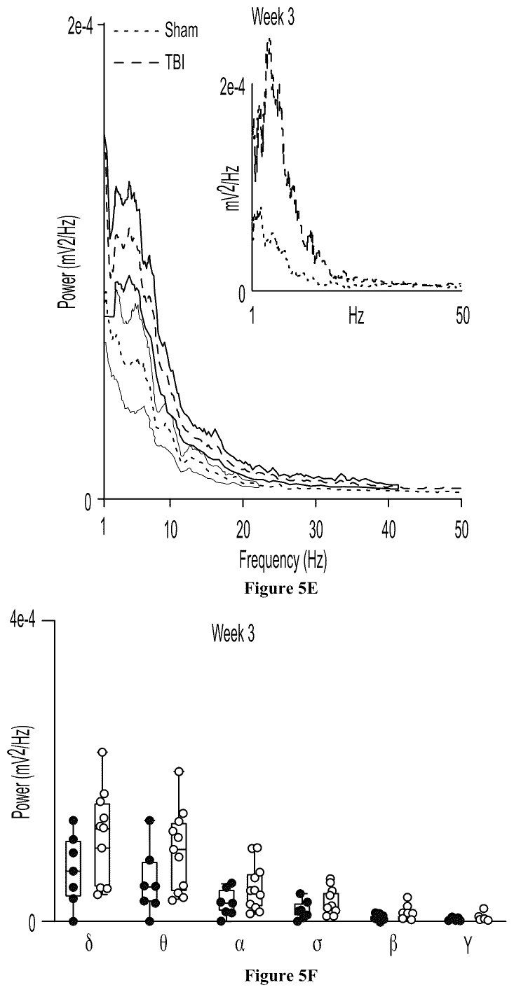

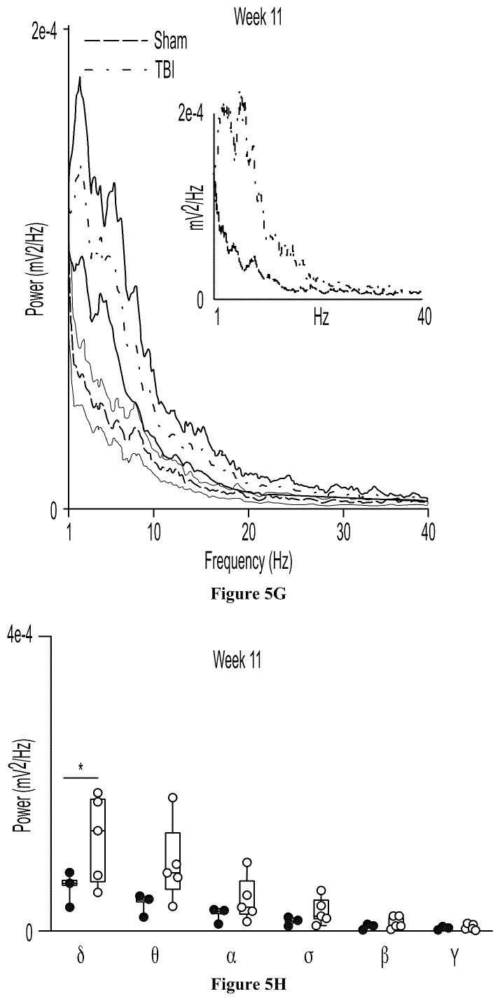

An anti-C1s antibody can comprise a heavy chain variable region comprising an amino acid sequence that is 85%, 86%, 87%, 88%, 89%, 90%, 91%, 92%, 93%, 94%, 95%, 96%, 97%, 98%, 99%, or 100% identical to the amino acid sequence set forth in SEQ ID NO:82 and depicted in FIG. 5 (VH variant 4).

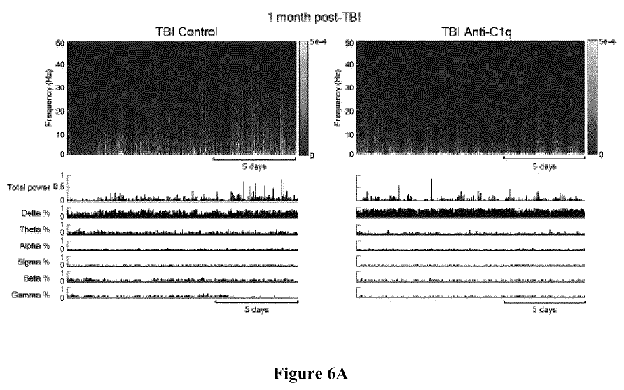

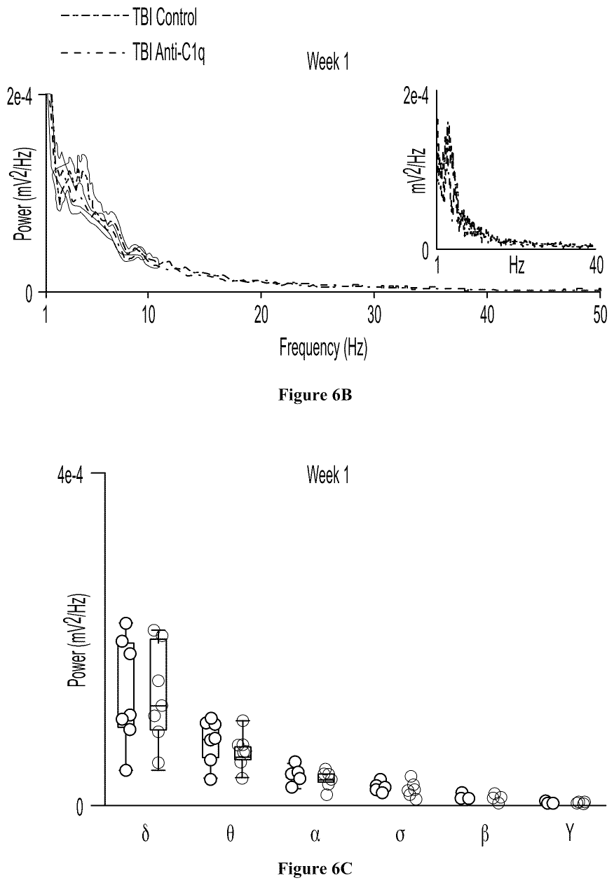

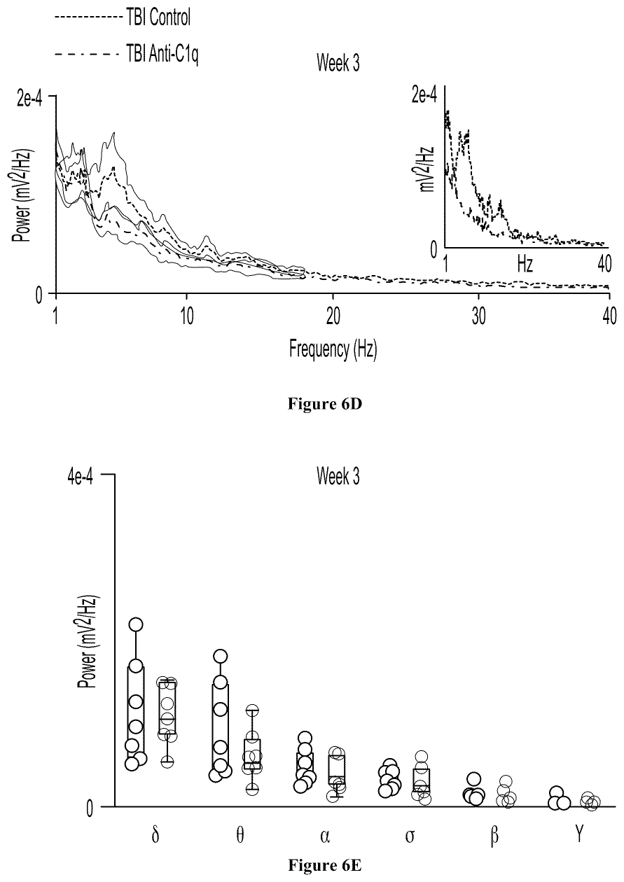

An anti-C1s antibody can comprise a light chain variable region comprising an amino acid sequence that is 85%, 86%, 87%, 88%, 89%, 90%, 91%, 92%, 93%, 94%, 95%, 96%, 97%, 98%, 99%, or 100% identical to the amino acid sequence set forth in SEQ ID NO:83 and depicted in FIG. 6 (VK variant 1).

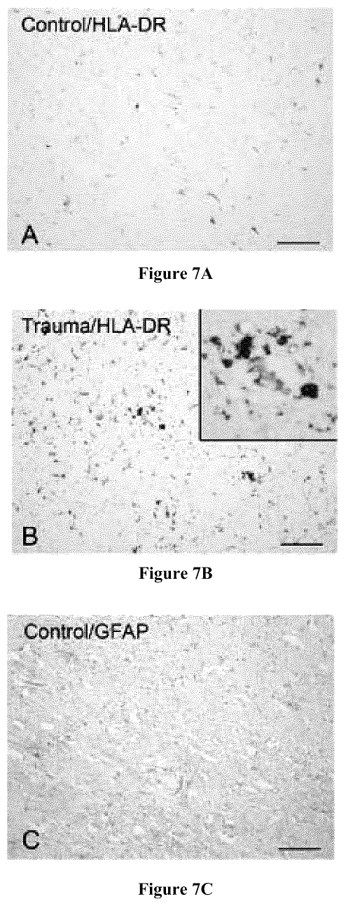

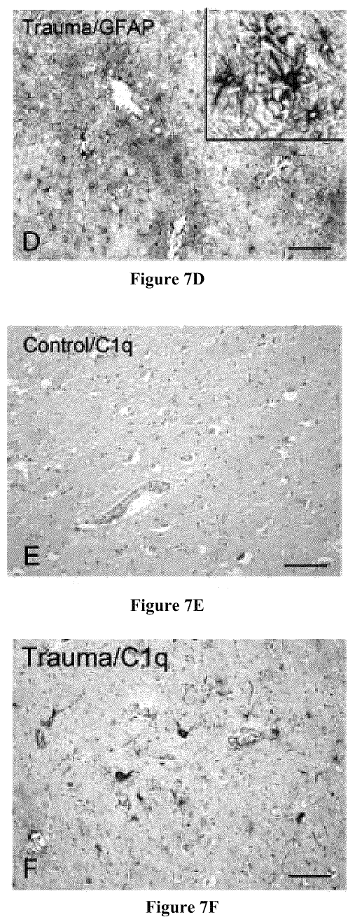

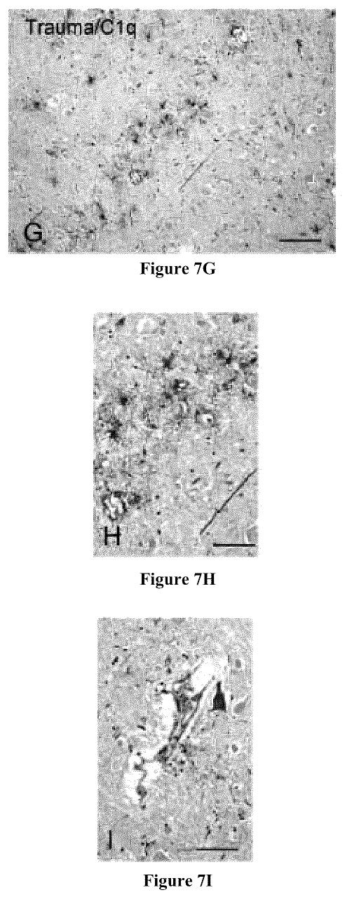

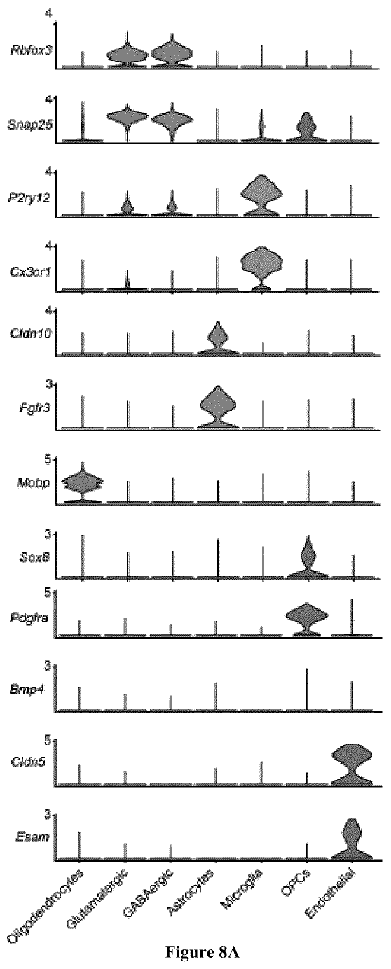

An anti-C1s antibody can comprise a light chain variable region comprising an amino acid sequence that is 85%, 86%, 87%, 88%, 89%, 90%, 91%, 92%, 93%, 94%, 95%, 96%, 97%, 98%, 99%, or 100% identical to the amino acid sequence set forth in SEQ ID NO:84 and depicted in FIG. 7 (VK variant 2).

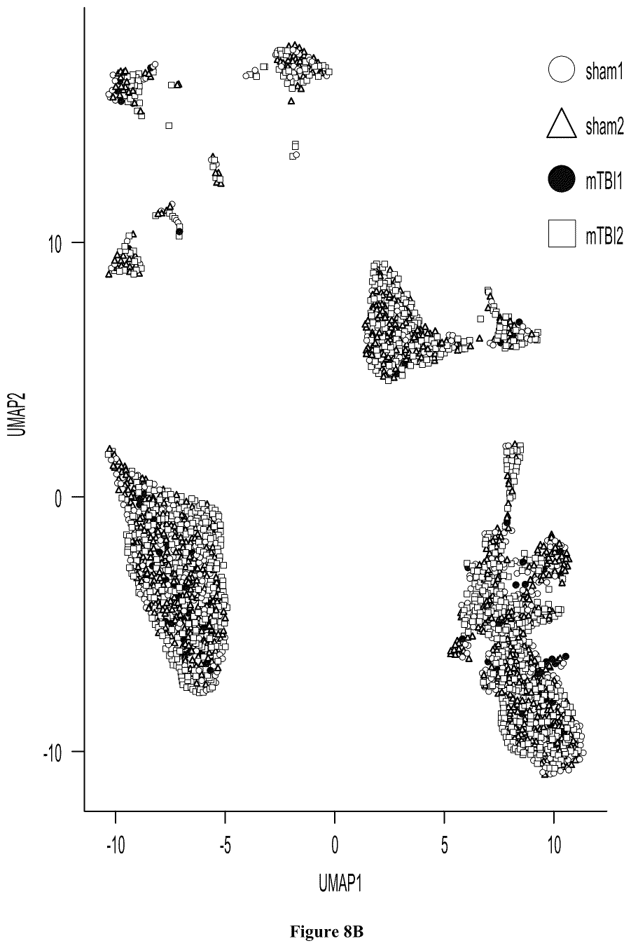

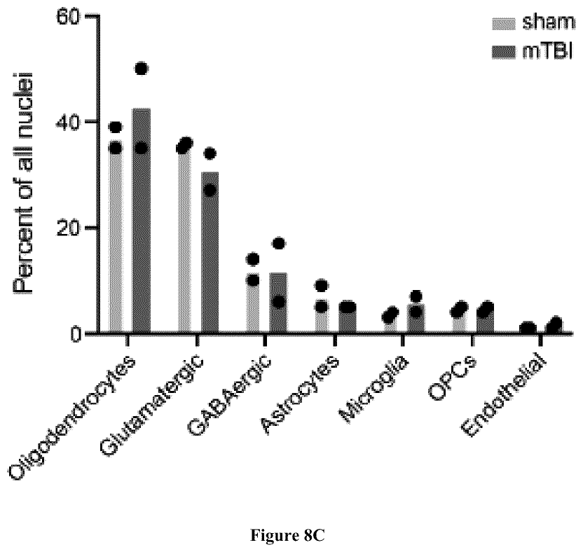

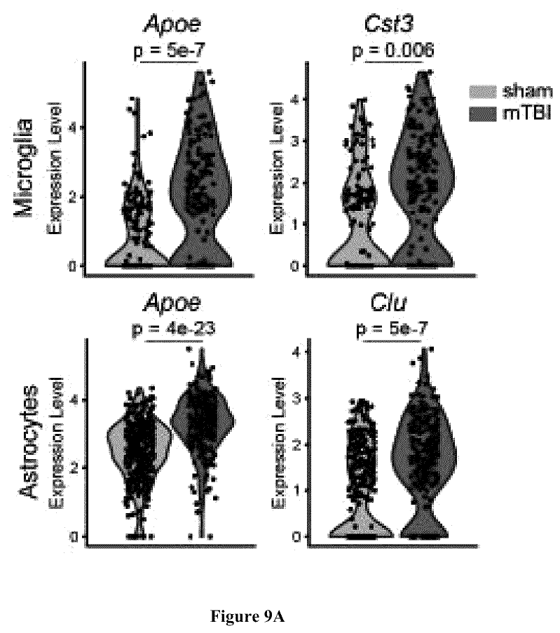

An anti-C1s antibody can comprise a light chain variable region comprising an amino acid sequence that is 85%, 86%, 87%, 88%, 89%, 90%, 91%, 92%, 93%, 94%, 95%, 96%, 97%, 98%, 99%, or 100% identical to the amino acid sequence set forth in SEQ ID NO:85 and depicted in FIG. 8 (VK variant 3).

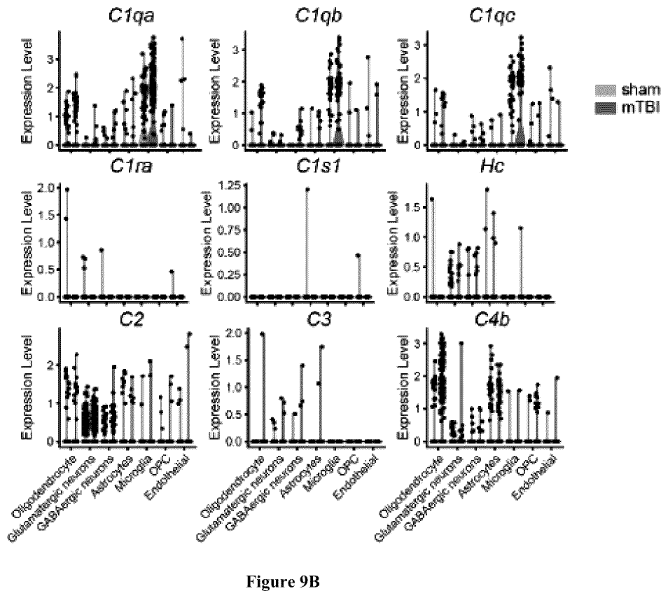

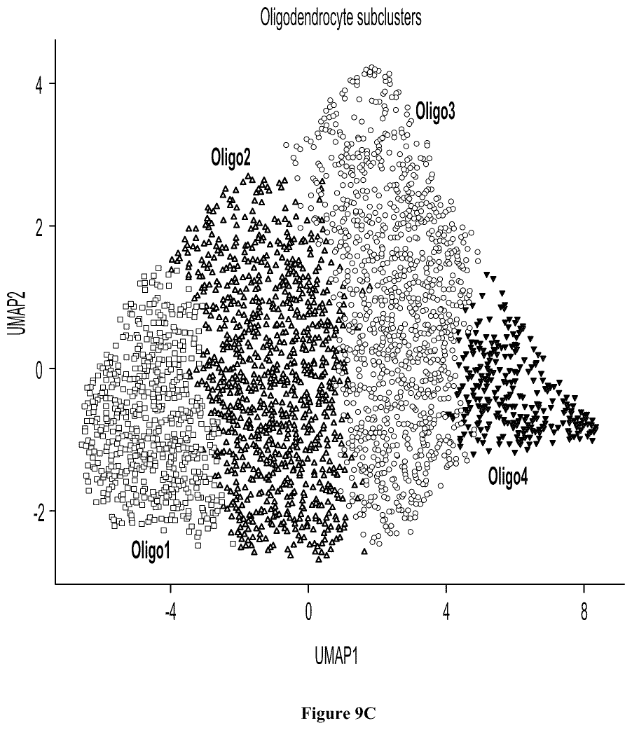

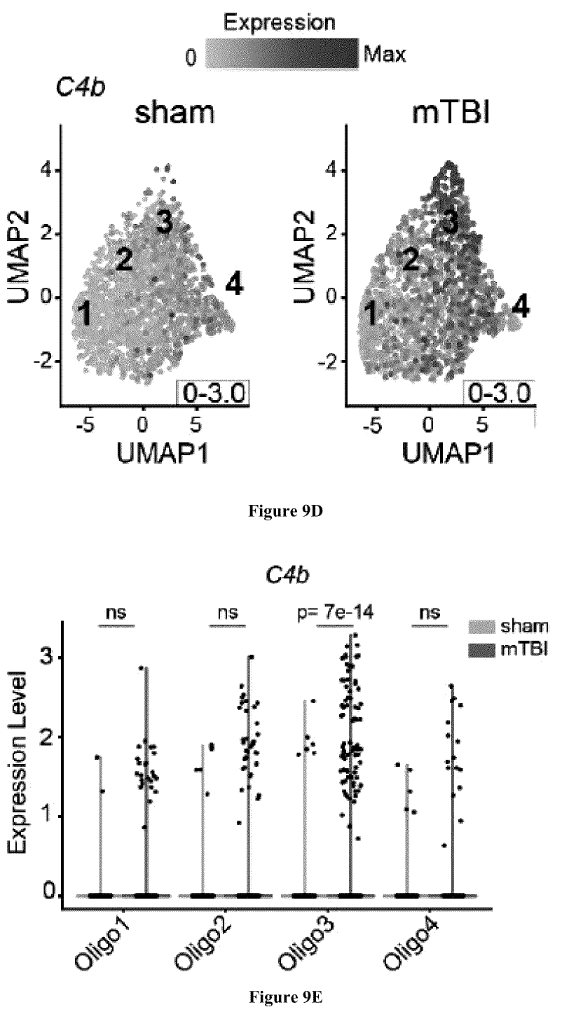

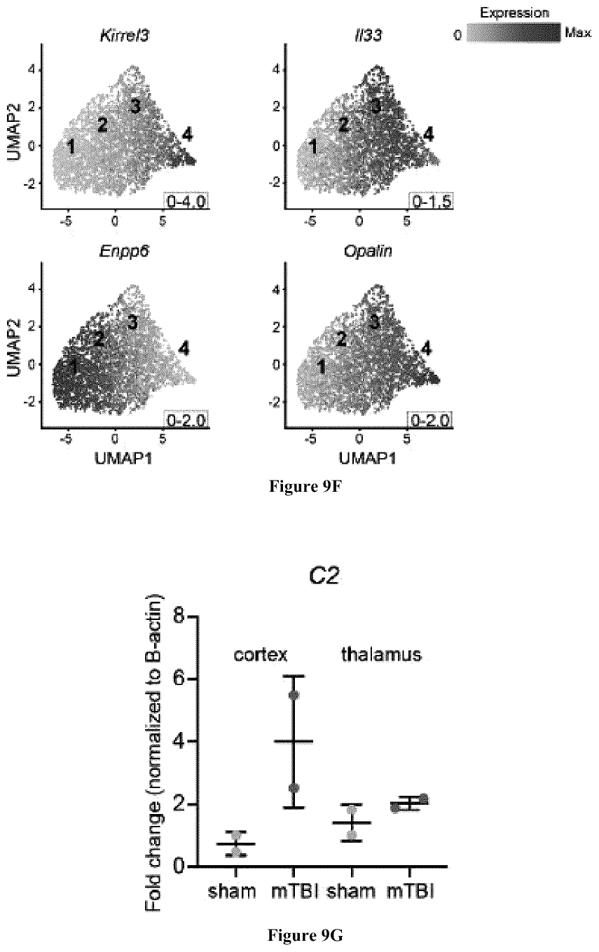

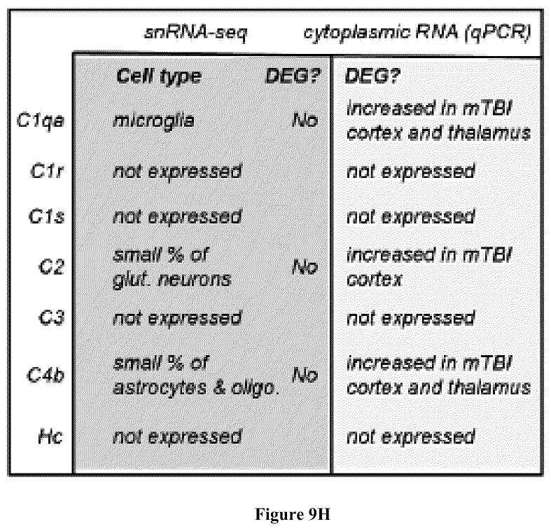

An anti-C1s antibody can comprise a heavy chain variable region comprising 1, 2, 3, 4, 5, 6, 7, 8, 9, 10, 11, or 12 of the framework (FR) amino acid substitutions, relative to the IPN003 parental antibody FR amino acid sequences, depicted in Table 3 (FIG. 9).

### Inhibition of Complement

A number of molecules are known that inhibit the activity of complement. In addition to known compounds, suitable inhibitors can be screened by methods described herein. As described above, normal cells can produce proteins that block complement activity, e.g., CD59, C1 inhibitor, etc. In some embodiments of the disclosure, complement is inhibited by upregulating expression of genes encoding such polypeptides.

Modifications of molecules that block complement activation are also known in the art. For example, such molecules include, without limitation, modified complement receptors, such as soluble CR1. The mature protein of the most common allotype of CR1 contains 1998 amino acid residues: an extracellular domain of 1930 residues, a transmembrane region of 25 residues, and a cytoplasmic domain of 43 residues. The entire extracellular domain is composed of 30 repeating units referred to as short consensus repeats (SCRs) or complement control protein repeats (CCPRs), each consisting of 60 to 70 amino acid residues. Recent data indicate that C1q binds specifically to human CR1. Thus, CR1 recognizes all three complement opsonins, namely C3b, C4b, and C1q. A soluble version of recombinant human CR1 (sCR1) lacking the transmembrane and cytoplasmic domains has been produced and shown to retain all the known functions of the native CR1. The cardioprotective role of sCR1 in animal models of ischemia/reperfusion injury has been confirmed. Several types of human C1q receptors (C1qR) have been described. These include the ubiquitously distributed 60- to 67-kDa receptor, referred to as cC1qR because it binds the collagen-like domain of C1q. This C1qR variant was shown to be calreticulin; a 126-kDa receptor that modulates monocyte phagocytosis. gC1qR is not a membrane-bound molecule, but rather a secreted soluble protein with affinity for the globular regions of C1q, and may act as a fluid-phase regulator of complement activation.

Decay accelerating factor (DAF) (CD55) is composed of four SCRs plus a serine/threonine-enriched domain that is capable of extensive O-linked glycosylation. DAF is attached to cell membranes by a glycosyl phosphatidyl inositol (GPI) anchor and, through its ability to bind C4b and C3b, it acts by dissociating the C3 and C5 convertases. Soluble versions of DAF (sDAF) have been shown to inhibit complement activation.

C1 inhibitor, a member of the “serpin” family of serine protease inhibitors, is a heavily glycosylated plasma protein that prevents fluid-phase C1 activation. C1 inhibitor regulates the classical pathway of complement activation by blocking the active site of C1r and C1s and dissociating them from C1q.

Peptide inhibitors of complement activation include C5a and other inhibitory molecules include Fucan.

### Nucleic Acids, Vectors and Host Cells

Antibodies suitable for use in the methods of the present disclosure may be produced using recombinant methods and compositions, e.g., as described in U.S. Pat. No. 4,816,567. In some embodiments, isolated nucleic acids having a nucleotide sequence encoding any of the antibodies of the present disclosure are provided. Such nucleic acids may encode an amino acid sequence containing the V_{L}/C_{L} and/or an amino acid sequence containing the V_{H}/C_{H}1 of the anti-C1q, anti-C1r or anti-C1s antibody. In some embodiments, one or more vectors (e.g., expression vectors) containing such nucleic acids are provided. A host cell containing such nucleic acid may also be provided. The host cell may contain (e.g., has been transduced with): (1) a vector containing a nucleic acid that encodes an amino acid sequence containing the V_{L}/C_{L} of the antibody and an amino acid sequence containing the V_{H}/C_{H}1 of the antibody, or (2) a first vector containing a nucleic acid that encodes an amino acid sequence containing the VL/CL of the antibody and a second vector containing a nucleic acid that encodes an amino acid sequence containing the VH/CH1 of the antibody. In some embodiments, the host cell is eukaryotic, e.g., a Chinese Hamster Ovary (CHO) cell or lymphoid cell (e.g., Y0, NS0, Sp20 cell). In some embodiments, the host cell is a bacterium such as E. coli.

Methods of making an anti-C1q, anti-C1r or anti-C1s antibody are disclosed herein. The method includes culturing a host cell of the present disclosure containing a nucleic acid encoding the anti-C1q, anti-C1r or anti-C1s antibody, under conditions suitable for expression of the antibody. In some embodiments, the antibody is subsequently recovered from the host cell (or host cell culture medium).

For recombinant production of a humanized anti-C1q, anti-C1r or anti-C1s antibody of the present disclosure, a nucleic acid encoding the antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColEl, pCR1, RP4, phage DNAs, and shuttle vectors such as pSA3 and pAT28. These and many other cloning vectors are available from commercial vendors such as BioRad, Stratagene, and Invitrogen.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-C1q, anti-C1r or anti-C1s antibody of the present disclosure.

Suitable host cells for cloning or expression of antibody-encoding vectors include prokaryotic or eukaryotic cells. For example, an anti-C1q, anti-C1r or anti-C1s antibody of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, N J, 2003), pp. 245-254, describing expression of antibody fragments in E. coli.). In other embodiments, the antibody of the present disclosure may be produced in eukaryotic cells, e.g., a Chinese Hamster Ovary (CHO) cell or lymphoid cell (e.g., Y0, NS0, Sp20 cell) (e.g., U.S. patent application Ser. No. 14/269,950, U.S. Pat. No. 8,981,071, Eur J Biochem. 1991 Jan. 1; 195(1):235-42). After expression, the antibody may be isolated from the bacterial cell paste in a soluble fraction and can be further purified.

### Conditions of Interest

Epilepsy is a group of neurological disorders in which nerve cell activity in the brain becomes disrupted, causing seizures or periods of unusual behavior, sensations and sometimes loss of consciousness. Epileptic seizures are episodes that can vary from brief and nearly undetectable to long periods of vigorous shaking. In epilepsy, seizures tend to recur, and have no immediate underlying cause. The cause of most cases of epilepsy is unknown, although some people develop epilepsy as the result of brain injury (e.g., TBI), stroke, brain tumor, and substance use disorders. Genetic mutations are linked to a small proportion of the disease. Epileptic seizures are the result of excessive and abnormal cortical nerve cell activity in the brain. The diagnosis typically involves ruling out other conditions that might cause similar symptoms such as fainting. Additionally, making the diagnosis involves determining if any other cause of seizures is present such as alcohol withdrawal or electrolyte problems. This may be done by imaging the brain and performing blood tests. Epilepsy can often be confirmed with an electroencephalogram (EEG) but a normal test does not rule out the condition. Other brain imaging technique may also detect a form of epilepsy, such as functional magnetic resonance imaging (fMRI), magnetic resonance spectroscopy (MRS), positron emission tomography (PET), and single-photon emission computed tomography (SPECT).

Epilepsy may occur as a result of a number of other conditions including tumors, strokes, head trauma, previous infections of the central nervous system, genetic abnormalities, and as a result of brain damage around the time of birth. There are several types of epilepsy, each with different causes, symptoms, and treatments. The two broad types of epilepsy are idiopathic (genetic causes), and symptomatic or cryptogenic (presumed symptomatic, cause unknown). In idiopathic generalized epilepsy, there is often, but not always, a family history of epilepsy. Idiopathic generalized epilepsy tends to appear during childhood or adolescence, although it may not be diagnosed until adulthood. In this type of epilepsy, no nervous system (brain or spinal cord) abnormalities, other than the seizures, can be identified on either an EEG or imaging studies (MRI). The brain is structurally normal on a brain magnetic resonance imaging (MRI) scan, although special studies may show a scar or subtle change in the brain that may have been present since birth. People with idiopathic generalized epilepsy have normal intelligence and the results of the neurological exam and MRI are usually normal. The results of the electroencephalogram (EEG—a test which measures electrical impulses in the brain) may show epileptic discharges affecting a single area or multiple areas in the brain (so called generalized discharges). The types of seizures affecting patients with idiopathic generalized epilepsy may include: Myoclonic seizures (sudden and very short duration jerking of the extremities), Absence seizures (staring spells), and/or Generalized tonic-clonic seizures (grand mal seizures). Idiopathic generalized epilepsy is usually treated with medications. Some people outgrow this condition and stop having seizures, as is the case with childhood absence epilepsy and a large number of patients with juvenile myoclonic epilepsy.

Idiopathic partial epilepsy begins in childhood (between ages 5 and 8) and may be part of a family history. Also known as benign focal epilepsy of childhood (BFEC), this is considered one of the mildest types of epilepsy. It is almost always outgrown by puberty and is never diagnosed in adults. Seizures tend to occur during sleep and are most often simple partial motor seizures that involve the face and secondarily generalized (grand mal) seizures. This type of epilepsy is usually diagnosed with an EEG.

Symptomatic generalized epilepsy is caused by widespread brain damage. Injury during birth is the most common cause of symptomatic generalized epilepsy. In addition to seizures, these patients often have other neurological problems, such as mental retardation or cerebral palsy. Specific, inherited brain diseases, such as adrenoleukodystrophy (ADL) or brain infections (such as meningitis and encephalitis) can also cause symptomatic generalized epilepsy. When the cause of symptomatic general epilepsy cannot be identified, the disorder may be referred to as cryptogenic epilepsy. These epilepsies include different subtypes—the most commonly known type is the Lennox-Gastaut syndrome. Multiple types of seizures (generalized tonic-clonic, tonic, myoclonic, tonic, atonic, and absence seizures) are common in these patients and can be difficult to control.

Symptomatic partial (or focal) epilepsy is the most common type of epilepsy that begins in adulthood, but it does occur frequently in children. This type of epilepsy is caused by a localized abnormality of the brain, which can result from traumatic brain injury, strokes, tumors, trauma, congenital (present at birth) brain abnormality, scarring or “sclerosis” of brain tissue, cysts, or infections. Sometimes these brain abnormalities can be seen on MRI scans, but often they cannot be identified, despite repeated attempts, because they are microscopic.

One example of symptomatic partial (or focal) epilepsy is temporal lobe epilepsy (TLE), which is a group of disorders that predominately involves dysregulation of amygdalo-hippocampal function caused by neuronal hyper-excitability. Medial TLE (MTLE) in particular, is perhaps the best-characterized electroclinical syndrome of all the epilepsies and is the most frequent form of focal epilepsy in adults. At least 70% of patients presenting with MTLE are resistant to currently available medication. The inherent potential for the temporal lobe to be predisposed to focal seizures is based on the unique anatomic-functional networks that involve the amygdalo-hippocampal complex and entorhinal cortex. Most patients with refractory TLE display severe unilateral hippocampal atrophy, so-called hippocampal sclerosis (HS), histopathologically characterized by segmental neuronal cell loss in the CA1 and CA4 subfields, astrogliosis, granule cell dispersion and axonal reorganization. Although in most cases the etiology of TLE is unknown (idiopathic), the disorder is frequently associated with an initial precipitating injury including febrile seizures, trauma, stroke, brain infections or status epilepticus (SE). There is general agreement that such injuries can cause pathological changes in the brain that trigger the process of epileptogenesis and, after a latent period of months to years, lead to epilepsy. Beyond seizures, drug-resistant TLE is characterized by cognitive decline, especially involving memory functions, and by psychiatric co-morbidities. Behavioral deficits in TLE have a great impact on the burden of the disease, and often contribute much more than seizures per se to negatively impact on the patient's quality of life.

### Pharmaceutical Compositions and Administration

A complement inhibitor (e.g. an antibody) of the present disclosure may be administered in the form of pharmaceutical compositions.

Therapeutic formulations of an inhibitor (e.g., an antibody) of the disclosure may be prepared for storage by mixing the inhibitor having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, acetate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, proline and/or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Lipofections or liposomes may also be used to deliver an antibody or antibody fragment into a cell, wherein the epitope or smallest fragment which specifically binds to the binding domain of the target protein is preferred.

The inhibitor may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for administration may be sterile. This is readily accomplished by filtration through sterile filtration membranes.

Sustained-release preparations may be prepared. Suitable examples of sustained-release preparations include semipermeable matrices of solid hydrophobic polymers containing the inhibitor, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The antibodies and compositions of the present disclosure are typically administered by various routes, including, but not limited to, topical, parenteral, subcutaneous, intraperitoneal, intrapulmonary, intranasal, and intralesional administration. Parenteral routes of administration include intramuscular, intravenous, intra-arterial, intraperitoneal, intravitreal, intrathecal, or subcutaneous administration.

Pharmaceutical compositions may also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation may include other carriers, adjuvants, or non-toxic, nontherapeutic, non-immunogenic stabilizers, excipients and the like. The compositions may also include additional substances to approximate physiological conditions, such as pH adjusting and buffering agents, toxicity adjusting agents, wetting agents and detergents.

The composition may also include any of a variety of stabilizing agents, such as an antioxidant for example. When the pharmaceutical composition includes a polypeptide, the polypeptide may be complexed with various well-known compounds that enhance the in vivo stability of the polypeptide, or otherwise enhance its pharmacological properties (e.g., increase the half-life of the polypeptide, reduce its toxicity, enhance other pharmacokinetic and/or pharmacodynamic characteristics, or enhance solubility or uptake). Examples of such modifications or complexing agents include sulfate, gluconate, citrate and phosphate. The polypeptides of a composition may also be complexed with molecules that enhance their in vivo attributes. Such molecules include, for example, carbohydrates, polyamines, amino acids, other peptides, ions (e.g., sodium, potassium, calcium, magnesium, manganese), and lipids. Further guidance regarding formulations that are suitable for various types of administration may be found in Remington's Pharmaceutical Sciences, Mace Publishing Company, Philadelphia, Pa., 17th ed. (1985). For a brief review of methods for drug delivery, see, Langer, Science 249:1527-1533 (1990).

Toxicity and therapeutic efficacy of the active ingredient may be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it may be expressed as the ratio LD50/ED50. Compounds that exhibit large therapeutic indices are preferred.

The data obtained from cell culture and/or animal studies may be used in formulating a range of dosages for humans. The dosage of the active ingredient typically lines within a range of circulating concentrations that include the ED50 with low toxicity. The dosage may vary within this range depending upon the dosage form employed and the route of administration utilized.

The pharmaceutical compositions described herein may be administered in a variety of different ways. Examples include administering a composition containing a pharmaceutically acceptable carrier via oral, intranasal, rectal, intravitreal, topical, intraperitoneal, intravenous, intramuscular, subcutaneous, subdermal, transdermal, intrathecal, and intracranial methods.

For oral administration, the active ingredient may be administered in solid dosage forms, such as capsules, tablets, and powders, or in liquid dosage forms, such as elixirs, syrups, and suspensions. The active component(s) may be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents may be used to make compressed tablets. Both tablets and capsules may be manufactured as sustained release products to provide for continuous release of medication over a period of hours. Compressed tablets may be sugar coated or film coated to mask any unpleasant taste and protect the tablet from the atmosphere, or enteric-coated for selective disintegration in the gastrointestinal tract. Liquid dosage forms for oral administration may contain coloring and flavoring to increase patient acceptance.

Formulations suitable for parenteral administration include aqueous and non-aqueous, isotonic sterile injection solutions, which may contain antioxidants, buffers, bacteriostats, and solutes that render the formulation isotonic with the blood of the intended recipient, and aqueous and non-aqueous sterile suspensions that may include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for parenteral use are usually sterile. To the extent that a given compound must be synthesized prior to use, the resulting product is typically substantially free of any potentially toxic agents, particularly any endotoxins, which may be present during the synthesis or purification process. Compositions for parental administration are also typically substantially isotonic and made under GMP conditions.

The compositions of the disclosure may be administered using any medically appropriate procedure, e.g., intravascular (intravenous, intraarterial, intracapillary) administration, injection into the cerebrospinal fluid, intravitreal, topical, intracavity or direct injection in the brain. Intrathecal administration may be carried out through the use of an Ommaya reservoir, in accordance with known techniques. (F. Balis et al., Am J. Pediatr. Hematol. Oncol. 11, 74, 76 (1989).

Where the therapeutic agents are locally administered in the brain, one method for administration of the therapeutic compositions of the disclosure is by deposition into or near the site by any suitable technique, such as by direct injection (aided by stereotaxic positioning of an injection syringe, if necessary) or by placing the tip of an Ommaya reservoir into a cavity, or cyst, for administration. Alternatively, a convection-enhanced delivery catheter may be implanted directly into the site, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic composition to the brain and/or tumor mass. Such devices are described in U.S. Pat. No. 5,720,720, incorporated fully herein by reference.

The effective amount of a therapeutic composition given to a particular patient may depend on a variety of factors, several of which may be different from patient to patient. A competent clinician will be able to determine an effective amount of a therapeutic agent to administer to a patient. Dosage of the agent will depend on the treatment, route of administration, the nature of the therapeutics, sensitivity of the patient to the therapeutics, etc. Utilizing LD50 animal data, and other information, a clinician may determine the maximum safe dose for an individual, depending on the route of administration. Utilizing ordinary skill, the competent clinician will be able to optimize the dosage of a particular therapeutic composition in the course of routine clinical trials. The compositions may be administered to the subject in a series of more than one administration. For therapeutic compositions, regular periodic administration will sometimes be required, or may be desirable. Therapeutic regimens will vary with the agent; for example, some agents may be taken for extended periods of time on a daily or semi-daily basis, while more selective agents may be administered for more defined time courses, e.g., one, two three or more days, one or more weeks, one or more months, etc., taken daily, semi-daily, semi-weekly, weekly, etc.

Formulations may be optimized for retention and stabilization in the brain. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc., in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e., having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers may be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Methods of Treatment

The methods of the invention provide for modulating the immune response to epilepsy through administering agents that are inhibitors of complement. Epilepsy may be induced by traumatic brain injury (TBI), hypoxic brain injury, brain infection, stroke, or genetic syndrome. In preferred embodiments, the epilepsy is TBI-induced epilepsy. Without being bound by theory, immature astrocytes induce expression of C1q proteins in neurons during development. In patients with TLE, there is evidence of microglial activation within the hippocampus, suggesting an activated immune response. Inflammatory mediators such as complement factor are normally expressed at very low levels in healthy brain tissue but can be rapidly induced by a variety of insults to the brain such as infection, ischaemia, injury and seizure. Activation of C1q, C1r, and C1s contributes to the inflammatory response, which leads to synaptic loss, along with the generation and recurrence of seizures and seizure-related neuronal damage. Dysregulated persistent inflammation, blood-brain barrier damage, and uncontrolled seizures trigger the progression of TLE. During the developmental process of TLE, overexpression of C1q, C1r, and C1s can be coupled with a signal for complement activation, e.g., β-amyloid, APP, cytokines such as IFNγ, TNFα, and the like, also resulting in inflammation.

By administering agents that inhibit complement activation, synapses can be maintained that would otherwise be lost. Such agents include C1q, C1r, and C1s inhibitors, agents that upregulate expression of native complement inhibitors, agents that down-regulate C1q, C1r, or C1s synthesis in neurons, agents that block complement activation, agents that block the signal for complement activation, and the like.

By administering agents that inhibit complement activation, production of antigen-specific antibodies that mediate epilepsy, such as an idiopathic generalized epilepsy, idiopathic partial epilepsy, symptomatic generalized epilepsy or symptomatic partial epilepsy, can be reduced. Such agents include an anti-C1q, anti-C1r, or anti-C1s antibody inhibitor. Other agents may include inhibitors that upregulate expression of native complement, or agents that down-regulate C1q, C1r or C1s synthesis in cells, agents that block complement activation, agents that block the signal for complement activation, and the like.

The methods promote improved maintenance of neuronal function in conditions associated with synapse loss. The maintenance of neural connections provides for functional improvement in neurodegenerative disease relative to untreated patients. The prevention of synapse loss may comprise at least a measurable improvement relative to a control lacking such treatment over the period of 1, 2, 3, 4, 5, 6 days or at least one week, for example at least a 10% improvement in the number of synapses, at least a 20% improvement, at least a 50% improvement, or more.

Preferably, the agents of the present invention are administered at a dosage that decreases synapse loss while minimizing any side-effects. It is contemplated that compositions will be obtained and used under the guidance of a physician for in vivo use. The dosage of the therapeutic formulation will vary widely, depending upon the nature of the disease, the frequency of administration, the manner of administration, the clearance of the agent from the host, and the like.

The effective amount of a therapeutic composition to be given to a particular patient will depend on a variety of factors, several of which will be different from patient to patient. Utilizing ordinary skill, the competent clinician will be able to tailor the dosage of a particular therapeutic or imaging composition in the course of routine clinical trials.

Therapeutic agents, e.g., inhibitors of complement, activators of gene expression, etc. can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

The methods neutralize complement biological activity. The affected complement biological activity could be (1) C1q binding to an autoantibody, (2) C1q binding to C1r, (3) C1q binding to C1s, (4) C1q binding to IgM, (5) C1q binding to IgG, (6) C1q binding to phosphatidylserine, (7) C1q binding to pentraxin-3, (8) C1q binding to C-reactive protein (CRP), (9) C1q binding to globular C1q receptor (gC1qR), (10) C1q binding to complement receptor 1 (CR1), (11) C1q binding to beta-amyloid, (12) C1q binding to calreticulin, (13) C1q binding to apoptotic cells, or (14) C1q binding to B cells. The affected complement biological activity could further be (1) activation of the classical complement activation pathway, (2) activation of antibody and complement dependent cytotoxicity, (3) CH50 hemolysis, (4) synapse loss, (5) B-cell antibody production, (6) dendritic cell maturation, (7) T-cell proliferation, (8) cytokine production (9) microglia activation, (10) Arthus reaction, (11) phagocytosis of synapses or nerve endings, or (12) activation of complement receptor 3 (CR3/C3) expressing cells.

It is contemplated that compositions may be obtained and used under the guidance of a physician for in vivo use. The dosage of the therapeutic formulation may vary widely, depending upon the nature of the disease, the frequency of administration, the manner of administration, the clearance of the agent from the host, and the like.

The effective amount of a therapeutic composition given to a particular patient may depend on a variety of factors, several of which may be different from patient to patient. Utilizing ordinary skill, the competent clinician will be able to tailor the dosage of a particular therapeutic or imaging composition in the course of routine clinical trials.

Therapeutic agents, e.g., inhibitors of complement, activators of gene expression, etc. can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intratracheal, etc., administration. The active agent may be systemic after administration or may be localized by the use of regional administration, intramural administration, or use of an implant that acts to retain the active dose at the site of implantation.

### Compound Screening

In one aspect of the invention, candidate agents to be used as inhibitors are screened for the ability to modulate synapse loss. Such compound screening may be performed using an in vitro model, a genetically altered cell or animal, or purified protein. A wide variety of assays may be used for this purpose. In one embodiment, compounds that are predicted to be antagonists or agonists of complement, including specific complement proteins, e.g., C1q, and complement activating signals, e.g., β-amyloid, APP, etc. are tested in an in vitro culture system, as described below.

For example, candidate agents may be identified by known pharmacology, by structure analysis, by rational drug design using computer based modeling, by binding assays, and the like. Various in vitro models may be used to determine whether a compound binds to, or otherwise affects complement activity. Such candidate compounds are used to contact neurons in an environment permissive for synapse loss. Such compounds may be further tested in an in vivo model for an effect on synapse loss.

Screening may also be performed for molecules produced by astrocytes, e.g. immature astrocytes, which induce C1q expression in neurons. In such assays, co-cultures of neurons and astrocytes are assessed for the production or expression of molecules that induce C1q expression. For example, blocking antibodies may be added to the culture to determine the effect on induction of C1q expression in neurons.

Synapse loss is quantitated by administering the candidate agent to neurons in culture, and determining the presence of synapses in the absence or presence of the agent. In one embodiment of the invention, the neurons are a primary culture, e.g., of RGCs. Purified populations of RGCs are obtained by conventional methods, such as sequential immunopanning. The cells are cultured in suitable medium, which will usually comprise appropriate growth factors, e.g., CNTF; BDNF; etc. The neural cells, e.g., RCGs, are cultured for a period of time sufficient allow robust process outgrowth and then cultured with a candidate agent for a period of about 1 day to 1 week. In many embodiments, the neurons are cultured on a live astrocyte cell feeder in order to induce signaling for synapse loss. Methods of culturing astrocyte feeder layers are known in the art. For example, cortical glia can be plated in a medium that does not allow neurons to survive, with removal of non-adherent cells.

For synapse quantification, cultures are fixed, blocked and washed, then stained with antibodies specific synaptic proteins, e.g., synaptotagmin, etc. and visualized with an appropriate reagent, as known in the art. Analysis of the staining may be performed microscopically. In one embodiment, digital images of the fluorescence emission are with a camera and image capture software, adjusted to remove unused portions of the pixel value range and the used pixel values adjusted to utilize the entire pixel value range. Corresponding channel images may be merged to create a color (RGB) image containing the two single-channel images as individual color channels. Co-localized puncta can be identified using a rolling ball background subtraction algorithm to remove low-frequency background from each image channel. Number, mean area, mean minimum and maximum pixel intensities, and mean pixel intensities for all synaptotagmin, PSD-95, and colocalized puncta in the image are recorded and saved to disk for analysis.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

Compounds that are initially identified by any screening methods can be further tested to validate the apparent activity. The basic format of such methods involves administering a lead compound identified during an initial screen to an animal that serves as a model for humans and then determining the effects on synapse loss. The animal models utilized in validation studies generally are mammals. Specific examples of suitable animals include, but are not limited to, primates, mice, and rats.

### Combination Treatments

The complement inhibitors of the present disclosure may be used, without limitation, conjointly with any additional treatment for epilepsy, such as idiopathic generalized epilepsy, idiopathic partial epilepsy, symptomatic generalized epilepsy or symptomatic partial epilepsy.

In some embodiments, an antibody, antibody fragment and/or antibody derivative disclosed herein is administered in combination with a second inhibiting anti-complement factor antibody, such as an anti-C1q or anti-C1r antibody, or anti-C1s antibody. In some embodiments, an antibody is administered with a second and a third inhibiting anti-complement factor antibody, such as an anti-C1s antibody, an anti-C1q antibody, and/or an anti-C1r antibody.

In some embodiments, the inhibitors of this disclosure are administered in combination with an inhibitor of antibody-dependent cellular cytotoxicity (ADCC). ADCC inhibitors may include, without limitation, soluble NK cell inhibitory receptors such as the killer cell Ig-like receptors (KIRs), which recognize HLA-A, HLA-B, or HLA-C and C-type lectin CD94/NKG2A heterodimers, which recognize HLA-E (see, e.g., López-Botet M., T. Bellón, M. Llano, F. Navarro, P. Garcia & M. de Miguel. (2000), Paired inhibitory and triggering NK cell receptors for HLA class I molecules. Hum. Immunol. 61: 7-17; Lanier L. L. (1998) Follow the leader: NK cell receptors for classical and nonclassical MHC class I. Cell 92: 705-707.), and cadmium (see, e.g., Immunopharmacology 1990; Volume 20, Pages 73-8).

In some embodiments, the antibodies, antibody fragments and/or antibody derivatives of this disclosure are administered in combination with an inhibitor of the alternative pathway of complement activation. Such inhibitors may include, without limitation, factor B blocking antibodies, factor D blocking antibodies, soluble, membrane-bound, tagged or fusion-protein forms of CD59, DAF, CR1, CR2, Crry or Comstatin-like peptides that block the cleavage of C3, non-peptide C3aR antagonists such as SB 290157, Cobra venom factor or non-specific complement inhibitors such as nafamostat mesilate (FUTHAN; FUT-175), aprotinin, K-76 monocarboxylic acid (MX-1) and heparin (see, e.g., T. E. Mollnes & M. Kirschfink, Molecular Immunology 43 (2006) 107-121).

In some embodiments, the antibodies, antibody fragments and/or antibody derivatives of this disclosure are administered in combination with an inhibitor of the interaction between the autoantibody and its autoantigen. Such inhibitors may include purified soluble forms of the autoantigen, or antigen mimetics such as peptide or RNA-derived mimotopes, including mimotopes of the AQP4 antigen. Alternatively, such inhibitors may include blocking agents that recognize the autoantigen and prevent binding of the autoantibody without triggering the classical complement pathway. Such blocking agents may include, e.g., autoantigen-binding RNA aptamers or antibodies lacking functional C1q binding sites in their Fc domains (e.g., Fab fragments or antibody otherwise engineered not to bind C1q).

### Kits

The invention also provides kits containing antibodies, antibody fragments, and/or antibody derivatives of this disclosure. Kits of the invention include one or more containers comprising a purified anti-C1s, anti-C1q, or anti-C1r antibody of the present disclosure and instructions for use in accordance with methods known in the art. Generally, these instructions comprise a description of administration of the inhibitor to treat or diagnose epilepsy, such as an idiopathic generalized epilepsy, idiopathic partial epilepsy, symptomatic generalized epilepsy or symptomatic partial epilepsy. The kit may further comprise a description of selecting an individual suitable for treatment based on identifying whether that individual has the disease and the stage of the disease.

The instructions generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert may indicate that the composition is used for treating TBI-induced epilepsy. Instructions for TBI-induced epilepsy may be provided for practicing any of the methods described herein.

The label or package insert may indicate that the composition is used for treating TLE. TLE instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is an inhibitor of classical complement pathway. The container may further comprise a second pharmaceutically active agent.

Kits may optionally provide additional components such as buffers and interpretive information. Normally, the kit comprises a container and a label or package insert(s) on or associated with the container.

### Diagnostic Uses

While some people with epileptic seizures have abnormal EEGs, many do not. There are a number of additional tests that help identify the type of seizure and its effects. These include complete neurological consultation for epilepsy and related conditions, neurophysiological tests, including routine EEGs and outpatient and inpatient video-EEG monitoring, long-term inpatient video-EEG monitoring with scalp or intracranial electrodes, neuroimaging (e.g., MRI, MRS, PET, fMRI), neuropsychology, and speech and auditory processing evaluations.

For example, temporal lobe epilepsy is the most common form of partial or localization-related epilepsy. In general terms, there are two types of temporal lobe epilepsy; one involves the medial or internal structures of the temporal lobe, while the second, called neocortical temporal lobe epilepsy, involves the outer portion of the temporal lobe. It is important to understand several features useful for determining a subject's risk of developing temporal lobe epilepsy (TLE). One feature of TLE is simple focal seizures without loss of awareness (with or without aura) or focal dyscognitive seizures (with loss of awareness). Loss of awareness occurs during a focal dyscognitive seizure when the seizure spreads to involve both temporal lobes. In epidemiology terms, focal epilepsy is often of temporal lobe origin but the true prevalence of TLE is not known. With respect to presentation, aura occurs in the majority of temporal lobe seizures. The majority of auras and automatisms last a very short period—seconds or 1 to 2 minutes. Auras may cause sensory, autonomic or psychic symptoms. Somatosensory and special sensory phenomena include olfactory, auditory and gustatory illusions, along with hallucinations. Patients may report distortions of shape, size and distance of objects. Such visual illusions differ from the visual hallucinations associated with occipital lobe seizure in that there is no formed visual image. For example, objects may appear smaller or larger than usual. In addition, vertigo may occur with seizures in the posterior superior temporal gyrus. Psychic phenomena includes the feeling of déjà vu (familiarity) or jamais vu (unfamiliarity), depersonalisation (e.g., feeling of detachment from oneself) or derealisation (surroundings appear unreal), fear or anxiety, and patients may describe seeing their own body from outside. Autonomic phenomena include changes in heart rate and sweating. Patients may experience an epigastric fullness sensation or nausea.

Following the aura, a temporal lobe focal dyscognitive seizure begins with a wide-eyed, motionless stare, dilated pupils and behavioral arrest. Lip-smacking, chewing and swallowing may be noted. Manual automatisms or unilateral dystonic posturing of a limb may also occur. A focal dyscognitive seizure may evolve to a generalized tonic-clonic (GTC) seizure. Patients usually experience a postictal period of confusion. The postictal phase may last for several minutes. Amnesia occurs during a focal dyscognitive seizure because of bilateral hemispheric involvement.

The possible underlying causes of TLE include past infections (e.g., herpes encephalitis or bacterial meningitis), traumatic brain injury, head injury producing contusion or haemorrhage that results in encephalomalacia or cortical scarring, hypoxic brain injury, brain infection, stroke hamartomas, gliomas, genetic syndrome, vascular malformations (e.g., arteriovenous malformation, cavernous angioma), cryptogenic (a cause is presumed but has not been identified), or idiopathic. Other underlying causes of TLE include Hippocampal sclerosis produced from mesial temporal lobe epilepsy, which begins in late childhood, then remits but reappears in adolescence or early adulthood in a refractory form. In addition, febrile seizures may lead to TLE, as some children with complex febrile convulsions appear to be at risk of developing TLE in later life.

Differential diagnosis is sometimes used in diagnosing and assessing persons at risk of TLE. Some features of differential diagnosis used in TLE diagnosis include excessive daytime somnolence (e.g., due to sleep apnea or narcolepsy), periodic limb movement disorder, tardive dyskinesia and occipital lobe epilepsy, which may spread to the temporal lobe and be clinically indistinguishable from a temporal lobe seizure. Psychogenic seizures, whereby patients with psychogenic seizures may also have epileptic seizures are also used in a differential diagnosis. Absence seizures, dyscognitive seizures, and frontal lobe focal dyscognitive seizures are also used in differential diagnosis of TLE. Absence seizures are characterized by an abrupt onset with no aura, usually last for less than 30 seconds, have no postictal confusion and are not associated with complex automatisms. Focal dyscognitive seizures are usually preceded by a distinct aura, last longer than a minute, and have a period of postictal confusion. Frontal lobe focal dyscognitive seizures appear in clusters of brief seizures with abrupt onset and ending. There is minimal postictal state and they may cause behavioral changes with vocalizations and complex motor and sexual automatisms. Differentiating from TLE may require electroencephalograph (EEG) localization.

Furthermore, with respect to diagnosis and traditional methods for determining a subject's risk of developing temporal lobe epilepsy, MRI is generally the neuroimaging diagnostic of choice. Routine MRI of the brain using certain labels will detect lesions (for example small tumors, vascular malformations and cortical dysplasia) that are not detected by computed tomography (CT). For example, one detectable label is interictal [¹⁸F]fluorodeoxyglucose-positron emission tomography (¹⁸FDG-PET), which has a sensitivity of 60-90%. Another detectable label is arterial spin labeling (ASL), which is capable of quantifying local cerebral blood flow by measuring the inflow of magnetically labeled arterial blood into the target region.

MRI carried out for the assessment of drug-resistant epilepsy requires specialized protocols. For example, hippocampal sclerosis is characterized by neuronal loss and gliosis. HS is the most common pathologic substrate of surgically treated epilepsy in adults and is seen in 67% of patients. In patients with newly diagnosed epilepsy, it has been reported in 1.5-3% of adults. When evaluating the medial temporal structures (hippocampus, amygdala, entorhinal cortex, and parahippocampal gyrus), MRI is used evaluate the size, signal, shape, and dual pathology (SSSD). The typical MRI findings of HS include atrophy of the hippocampus on T1-weighted SPGR (typically seen in 90-95% of cases). The atrophy is most prominent in the hippocampal body.

Using Fluid-attenuated inversion recovery (FLAIR) imaging, increased signal is observed in the hippocampus. FLAIR is ideally suited to detect signal changes in the hippocampus, since gliotic changes have increased water content appearing as increased signal on T2-weighted MRI. The FLAIR sequence nulls the increased signal intensity of the cerebrospinal fluid (CSF) in the temporal horn of the lateral ventricle and the choroidal fissure that can dwarf the increased signal in the hippocampus on a conventional thin-slice T2-weighted spin echo image. The baseline signal of the hippocampus on FLAIR MRI is greater than that of the cortex. In children, HS is observed in 21% of patients with newly diagnosed TLE and in up to 57% of patients with intractable TLE. More common findings in children with intractable TLE include MCDs and developmental tumors.

CT scanning has a role in the urgent assessment of seizures, or when MRI is contraindicated (for example when patients have pacemakers or metallic implants). A non-contrast CT scan will fail to identify some vascular lesions and tumors. CT has only a limited role in the assessment of intractable epilepsy. Electrocardiography (ECG) may also be carried out in the assessment of all patients with altered consciousness, particularly those in older age groups, when disorders of cardiac rhythm may simulate epilepsy. Twenty-four hour ambulatory ECG and other cardiovascular tests (including implantable loop devices) may also be helpful. Positron emission tomography (PET) using radioisotope fluorodeoxyglucose (18^{F}) (FDG-PET) as a detectable label is useful when the MRI result is normal. Interictal EEG is also used to obtain recording from scalp electrodes, as one third of patients with TLE have bilateral, independent, temporal interictal epileptiform abnormalities. In addition, single-photon emission computed tomography (SPECT) is useful for candidates for surgical intervention, while video-EEG telemetry is used as part of the pre-surgical evaluation. It is also used if the diagnosis of TLE is still uncertain.

The present invention provides, in part, methods of determining a subject's risk of developing epilepsy, such as an idiopathic generalized epilepsy, idiopathic partial epilepsy, symptomatic generalized epilepsy or symptomatic partial epilepsy, comprising: administering an anti-C1q, anti-C1r, or anti-C1s antibody to the subject, wherein the anti-C1q, anti-C1r, or anti-C1s is coupled to a detectable label; detecting the detectable label to measure the amount or location of C1q, C1r, or C1s in the subject; and comparing the amount or location of one or more of C1q, C1r, or C1s to a reference, wherein the risk of developing epilepsy, such as an idiopathic generalized epilepsy, idiopathic partial epilepsy, symptomatic generalized epilepsy or symptomatic partial epilepsy is characterized based on the comparison of the amount or location of one or more of C1q, C1r, or C1s to the reference.

An exemplary method for detecting the level of C1q, C1r, or C1s, and thus useful for classifying whether a sample is associated with epilepsy, such as an idiopathic generalized epilepsy, idiopathic partial epilepsy, symptomatic generalized epilepsy or symptomatic partial epilepsy or a clinical subtype thereof involves obtaining a biological sample from a test subject and contacting the biological sample with an antibody capable of detecting C1q, C1r, or C1s such that the level of C1q, C1r, or C1s is detected in the biological sample. In certain instances, the statistical algorithm is a single learning statistical classifier system. For example, a single learning statistical classifier system can be used to classify a sample as a C1q, C1r, or C1s sample based upon a prediction or probability value and the presence or level of C1q, C1r, or C1s. The use of a single learning statistical classifier system typically classifies the sample as a C1q, C1r, or C1s sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

In one embodiment, the methods further involve obtaining a control biological sample (e.g., biological sample from a subject who does not have a condition or disorder mediated by C1q, C1r, or C1s), a biological sample from the subject during remission or before developing a condition or disorder mediated by C1q, C1r, C1s, or a biological sample from the subject during treatment for developing a condition or disorder mediated by C1q, C1r, or C1s.

An exemplary method for detecting the presence or absence of C1q, C1r, or C1s is anti-C1q, anti-C1r, or anti-C1s antibody to the subject, wherein the anti-C1q, anti-C1r, or anti-C1s antibody is coupled to a detectable label. In some embodiments, the detectable label comprises a nucleic acid, oligonucleotide, enzyme, radioactive isotope, biotin or a fluorescent label. In some embodiments, the detectable label is detected using an imaging agent for x-ray, CT, MRI, ultrasound, PET and SPECT. In some embodiments, the fluorescent label is selected from fluorescein, rhodamine, cyanine dyes or BODIPY.

It is to be understood that one, some, or all of the properties of the various embodiments described herein may be combined to form other embodiments of the compositions and methods provided herein. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein. These and other aspects of the compositions and methods provided herein will become apparent to one of skill in the art.

### EXAMPLES

### Example 1: Materials and Methods

### Animals

Adult (P30-P180) male CD1 mice were used for most experiments. Adult male Thy1-GCaMP6f mice (Tg(Thy1-GCaMP6f)GP5.17Dkim ISMR JAX: 025393; C57BL/6 congenic), wildtype C57BL/6 mice (ISMR JAX: 000664), and C1q null mice (C1qatm1Mjw, ISMR_APB: 1494; C57BL/6 congenic) were used for specific experiments.

### Controlled Cortical Impact (CCI)

We anesthetized mice with 2-5% isoflurane and placed them in a stereotaxic frame. We performed a 3 mm craniotomy over the right somatosensory cortex (S1) centered at −1 mm posterior from Bregma, +3 mm lateral from the midline. TBI was performed with a CCI device (Impact One Stereotaxic Impactor for CCI, Leica Microsystems) equipped with a metal piston using the following parameters: 3 mm tip diameter, 150 angle, depth 0.8 mm from the dura, velocity 3 m/s, and dwell time 100 ms. Sham animals received identical anesthesia and craniotomy, but the injury was not delivered.

### Immunostaining and Microscopy

We anesthetized mice with a lethal dose of Fatal-Plus (see the World Wide Web at drugs.com/vet/fatal-plus-solution.html) and perfused with 4% paraformaldehyde in 1×PBS. Serial coronal sections (30 μm thick) were cut on a Leica SM2000R sliding microtome. Sections were incubated with antibodies directed against C1q (1:700, rabbit, Abcam, ab182451, 640 AB_2732849), GFAP (1:1000, chicken, Abcam, ab4674, AB_304558), GFP (1:500, chicken, Aves Labs, AB_10000240), Ibal (1:500, rabbit, Wako, 019-19741, AB_839504), and NeuN (1:500, mouse, Millipore, MAB377, AB_2298772) overnight at 4° C. After wash, we incubated sections with Alexa Fluor-conjugated secondary antibodies (1:300, Thermo Fisher Scientific, A-11029) for two hours at room temperature. We mounted sections in an antifade medium (Vectashield) and imaged using a Biorevo BZ-9000 Keyence microscope at 10-20×. Confocal imaging was performed using a confocal laser scanning microscope (LSM880, Zeiss) equipped with a Plan Apochromat 10×/0.45 NA air or 63×/1.4 NA oil immersion objective lens. A multi-line Argon laser was used for 488 nm excitation of AlexaFluor488 and a HeNe laser was used for 561 nm excitation of AlexaFluor594.

### Immunostaining of Human Tissue

Formalin-fixed, paraffin-embedded tissue was sectioned at 6 μm and mounted on organosilane-coated slides (SIGMA, St. Louis, MO). Representative sections of specimens were processed for hematoxylin/eosin, as well as for immunocytochemistry. Immunocytochemistry for C1q (1:200, rabbit polyclonal; DAKO, Denmark), was carried out on a paraffin-embedded tissue as previously described. Sections were incubated for one hour at room temperature followed by incubation at 4° C. overnight with primary antibodies. Single-labeled immunocytochemistry was performed using Powervision method and 3,3-diaminobenzidine as chromogen. Sections were counterstained with hematoxylin. An extensive neuropathological protocol was used (based upon the recommendations of the Brain-Net Europe consortium; Acta Neuropathologica 115(5):497-507-2008), including markers such as pTau (AT8), β-amyloid, pTDP-43 and alpha-synuclein.

### Slice Preparation for Electrophysiology

We euthanized mice with 4% isoflurane, perfused with ice-cold sucrose cutting solution containing 234 mM sucrose, 11 mM glucose, 10 mM MgSO4, 2.5 mM KCl, 1.25 mM NaH2PO4, 0.5 mM CaCl2), and 26 mM NaHCO₃, equilibrated with 95% O2 and 5% CO2, pH 7.4, and decapitated. We prepared 250-μm thick horizontal slices for thalamic recordings, and coronal slices for neocortical recordings with a Leica VT1200 microtome (Leica Microsystems). Slices were incubated at 32° C. for one hour and then at 24-26° C. in artificial cerebrospinal fluid (ACSF) containing 126 mM NaCl, 10 mM glucose, 2.5 mM KCl, 2 mM CaCl2), 1.25 mM NaH2PO4, 1 mM MgSO4, and 26 mM NaHCO3, and equilibrated with 95% O2 and 5% CO2, pH 7.4. Thalamic slice preparations were performed as described.

### Patch-Clamp Electrophysiology

Recordings were performed as previously described. We visually identified S1, nRT, and VB neurons by differential contrast optics with an Olympus microscope and an infrared video camera. Recording electrodes made of borosilicate glass had a resistance of 2.5-4 MΩ when filled with intracellular solution. Access resistance was monitored in all the recordings, and cells were included for analysis only if the access resistance was <25 MΩ. Intrinsic and bursting properties and spontaneous excitatory postsynaptic currents (EPSCs) were recorded in the presence of picrotoxin (50 μM, Sigma) and the internal solution contained 120 mM potassium gluconate, 11 mM EGTA, 11 mM KCl, 10 mM HEPES, 1 mM CaCl2), and 1 mM MgCl2, pH adjusted to 7.4 with KOH (290 mOsm). We corrected the potentials for −15 mV liquid junction potential.

Spontaneous inhibitory postsynaptic currents (IPSCs) were recorded in the presence of kynurenic acid (2 mM, Sigma), and the internal solution contained 135 mM CsCl, 10 mM EGTA, 10 mM 685 HEPES, 5 mM Qx-314 (lidocaine N-ethyl bromide), and 2 mM MgCl2, pH adjusted to 7.3 with CsOH (290 mOsm).

### Single-Nucleus RNA-Seq

Tissue Dissection

We euthanized mice with 4% isoflurane, perfused with ice-cold 1×PBS, and decapitated. We prepared 300-μm thick coronal slices with a Leica VT1200 microtome (Leica Microsystems) and placed under a Zeiss SteREO Discovery.V8 stereoscopic microscope (Zeiss) for visually-guided micro-dissections of the nRT and the adjacent relay thalamic nuclei (as shown in FIG. 3A).

Two replicate sham and mTBI groups were collected for this study. Replicate 1 contained tissue from n=five sham mice and n=six mTBI mice. Replicate 2 contained tissue from n=four sham mice and n=four mTBI mice.

Single Nuclei Isolation

Nuclei were isolated from the nRT/thalamus and cortex as previously described (62 and dx.doi.org/10.17504/protocols.io.6t8herw). Briefly, the tissue was placed into a pre-chilled Dounce tissue grinder with 1 mL of homogenization buffer with 200 units of RNasin Plus Ribonuclease Inhibitor (Promega). Tissue samples were homogenized with 10 strokes of the loose “A” pestle and 15 strokes of the tight “B” sized pestle. The lysate was passed through a 40 μm FlowMi strainer and nuclei were pelted at 500 RCF at 4° C. A fraction of the resulting supernatant containing the cytoplasmic RNA was frozen for downstream analysis. Pelleted nuclei were resuspended in the homogenization buffer, purified using a iodixanol gradient, and immediately used for snRNA-seq. Excess nuclei were cryopreserved in BamBanker (Wako Chemicals).

Single Nucleus RNA Library Construction and Sequencing

SnRNA-seq libraries were processed using the Chromium Next GEM Single Cell 3′v3 library kit with Dual Indexes (10× Genomics) according to the manufacturer's specifications. For every sample, nuclei were diluted to 1,000 nuclei/μl in Nuclei Dilution Buffer, and 9,900 nuclei were loaded onto the Chromium, with a targeted recovery of 6,000 nuclei. Replicate 1 and 2 nuclei were processed on different Chromium runs. Libraries were pooled based on their molar concentrations and sequenced on an Illumina NovaSeq 6000 system using an S1 flow cell and a v1 300-cycle Reagent Kit with 28 cycles for read 1, 90 cycles for read 2, 10 cycles for index i7 and 10 cycles for index i5. Cell Ranger (4.0.0) (10× Genomics) was used to perform sample de-multiplexing, barcode processing and single-cell gene-UMI counting. Reads were mapped to mm10 (GENCODE vM23/Ensembl 98, from 10×). From replicate 1, we recovered 2,337 nuclei from sham mice with a mean reads per cell of 92,533 and 650 nuclei from mTBI mice with a mean read per nuclei of 162,363; from Replicate 2, we recovered 3,891 nuclei from sham mice with a mean reads per cell of 47,592 and 4,575 nuclei from TBI mice with a mean reads per cell of 41,658 median of 2,200 genes per cell. Raw data are deposited on GEO under accession number.

Analysis of Nuclei Clusters

After processing with CellRanger, data matrices were analyzed using Seurat. Prior to analysis, ambient RNA was removed using SoupX using the default parameters. Potential doublets were removed from each GEM reaction using DoubletFinder. In replicate 1, 90 doublets were removed from sham, 147 from TBI. In replicate 2, 28 doublets were removed from sham and 181 from TBI. Nuclei singlets by DoubletFinder were utilized for downstream analysis. In addition to removing doublets, we removed nuclei with more than 1% expression of mitochondrial RNAs. Expression was log scale normalized and the top 2000 features were used for PCA and downstream clustering and UMAP. Harmony was used to combine the separate replicates. After Harmony correction, we observed no differences between replicates. Clusters were called using FindClusters and were annotated manually using key lineage markers. GABAergic nuclei were subclustered again after selection for Slc17a7/Slc17a6 negative neurons. Differentially expressed genes (DEGs) were analyzed, both between clusters and between sham and mTBI using the FinderMarkers function. P-values were calculated using the Wilcoxon Rank Sum test. For visualization of expression on UMAP projects, RNA expression values were imputed using Markov Affinity-based Graph Imputation of cells (MAGIC).

Quantitative Real-Time PCR for Cytoplasmic RNA

Bulk cytoplasmic RNA was extracted from each replicate sample as previously described (62). Briefly, 150 μL of homogenate was mixed with 1.5 mL of Trizma (Zymo). The aqueous layer was retained, mixed with ethanol and loaded into a Zymo GC column (Zymo Quick RNA mini kit), following the manufacturer's specifications for Zymo. RNA concentration was quantified using a spectrophotometer. 200 ng of each sample were used as input for cDNA synthesis using the SuperScript III First-Strand Synthesis kit (Invitrogen) according to manufacturer's specification with random hex primers. Specific target cDNA were quantified using the SSO Advanced Universal Sybr Green Supermix (BioRad) according to manufacturer's specifications. Relative expression was calculated using the delta-delta CT method using Actin as an internal reference. Samples processed with no reverse transcriptase were used to determine background. C1qa-F 5′-ATGGAGACCTCTCAGGGATG-3′ (SEQ ID NO: 69), C1qa-R 5′-ATACCAGTCCGGATGCCAGC-3′ (SEQ ID NO: 70), Actin-F: 5′-ATACCAGTCCGGATGCCAGC-3′ (SEQ ID NO: 71), Actin-R: 5′-TCACCCACACTGTGCCCATCTACGA-3′ (SEQ ID NO: 72), C4b-F: 5′-GACAAGGCACCTTCAGAACC-3′ (SEQ ID NO: 73), C4b-R: 5′-CAGCAGCTTAGTCAGGGTTACA-3′ (SEQ ID NO: 74), Clra-F:5′-GCCATGCCCAGGTGCAAGATCAA-3′ (SEQ ID NO: 75), Clra-R: 5′-TGGCTGGCTGCCCTCTGATG-3′ (SEQ ID NO: 76), C1s1-F:5′-TGGACAGTGGAGCAACTCCGGT-3′ (SEQ ID NO: 77), C1s-R: 5′-GGTGGGTACTCCACAGGCTGGAA-3′ (SEQ ID NO: 78), C2-F:5′-CTCATCCGCGTTTACTCCAT-3′ (SEQ ID NO: 79), C2-R: 5′-TGTTCTGTTCGATGCTCAGG-3′ (SEQ ID NO: 80), C3-F: 5′-AGCAGGTCATCAAGTCAGGC-3′ (SEQ ID NO: 81), C3-R: 5′-GATGTAGCTGGTGTTGGGCT,-3′ (SEQ ID NO: 82) C4-F: 5′-ACCCCCTAAATAACCTGG-3′ (SEQ ID NO: 83), C4-R: 5′-CCTCATGTATCCTTTTTGGA-3′ (SEQ ID NO: 84), Hc-F: 5′-AGGGTACTTTGCCTGCTGAA-3 (SEQ ID NO: 85); Hc-R: 5′-TGTGAAGGTGCTCTTGGATG-3′ (SEQ ID NO: 86).

### Surgical Implantation of Devices for Simultaneous Recording of ECoG (Electrocorticography) and MUA (Multi-Unit Activity)

The devices for simultaneous ECoG, MUA recordings, and optical manipulations in freely behaving mice were all custom made in the Paz lab as described in. In general, recordings were optimized for assessment of somatosensory subnetworks (primary somatosensory cortex (S1), somatosensory ventrobasal thalamus (VB), and somatosensory reticular thalamic nucleus (nRT). We implanted cortical screws bilaterally over S1 (contralateral to injury: −0.5 mm posterior from Bregma, −3.25 mm lateral; ipsilateral: +1.0-1.4 mm anterior from Bregma, +2.5-3.0 mm lateral), centrally over PFC (+0.5 mm anterior from Bregma, 0 mm lateral), and in the right hemisphere over VI (−2.9 mm posterior from Bregma, +2.7 mm lateral). For MUA recordings in VB, we implanted electrodes at −1.65 mm posterior from Bregma, +1.75 mm lateral, with the tips of the optical fiber at 3.0 mm and two electrodes at 3.25 mm and 3.5 mm ventral to the cortical surface. For MUA recordings in nRT, we implanted electrodes at −1.4 mm posterior from Bregma, +2.1 mm lateral, with the tips of the optical fiber at 2.7 mm and two electrodes at 2.9 mm, and 3.0 mm ventral to the cortical surface, respectively.

### In Vivo Electrophysiology and Behavior

Non-chronic MUA electrophysiological recordings in freely behaving mice were performed as described using custom-made optrode devices. ECoG and thalamic LFP/MU (local field potentials/multiunit) signals were recorded using RZ5 (TDT) and sampled at 1221 Hz, with thalamic MUA signals sampled at 24 kHz. A video camera that was synchronized to the signal acquisition was used to continuously monitor the animals. We briefly anesthetized animals with 2% isoflurane at the start of each recording to connect for recording. Each recording trial lasted 15-60 min. To control for circadian rhythms, we housed our animals using a regular light/dark cycle and performed recordings between roughly 9:00 am and 6:00 pm. All the recordings were performed during wakefulness. We validated the location of the optrodes by histology after euthanasia in mice that did not experience sudden death and whose brains, we were able to recover and process.

### Surgical Implantation of Devices for Chronic ECoG Recordings

The wireless telemetry devices we used for chronic ECoG recordings were purchased from Data Sciences International (DSI). After performing controlled cortical impact surgery, we implanted cortical screws bilaterally over S1 as described above. The battery/transmitter device was placed under the skin over the right shoulder. We began recording mice as soon as they recovered from the surgery. Mice were singly housed in their home cages, which were placed over receivers that sent signals to an acquisition computer. ECoG signals were continuously recorded from up to eight mice simultaneously using Ponemah software (DSI) and sampled at 500 Hz.

### Statistical Analyses

All numerical values are given as means and error bars are standard error of the mean (SEM) unless stated otherwise. Parametric and non-parametric tests were chosen as appropriate and were reported in figure legends. Data analysis was performed with MATLAB (SCR_001622), GraphPad Prism 7/8 (SCR_002798), ImageJ (SCR_003070), Ponemah/NeuroScore (SCR_017107), pClamp (SCR_011323), and Spike2 (SCR 000903).

### Image Analysis and Cell Quantification

We selected regions of interest (ROIs) for S1, nRT, and VB from 10× Keyence microscope images opened in ImageJ (SCR 003070). To ensure that each ROI covered the same area on the ipsilateral and contralateral sides of the injury site, the first ROIs were duplicated and repositioned over the opposite hemisphere. The image was then converted to 8-bit. The upper threshold was adjusted to the maximum value of 255, and the lower threshold was increased from 0 until the pixel appearance most closely matched the fluorescence staining from the original image. A particle analysis was run on the ROIs using the same threshold boundaries for all sections with the same stain. An integrated density ratio was calculated for each brain region by dividing the ipsilateral integrated pixel density by the contralateral integrated pixel density. The integrated density ratios from three sections per animal were averaged to get a single average ratio per brain area for each animal.

nRT cell counts were performed on sections stained with NeuN. The nRT was outlined in ImageJ (SCR 003070) and we performed a manual cell count of neuronal cell bodies using the manual counter plugin.

### Analysis of Electrophysiological Properties

The input resistance (Rin) and membrane time constant (τm) were measured from the membrane hyperpolarizations in response to low intensity current steps (−20 to −60 pA). The reported rheobase averages and SEMs were calculated based on the current which first caused at least one action potential during the stimulus per recording. All data were analyzed using a Mann-Whitney test with α=0.05 (*p<0.05, **p<0.01, ***p<0.001, ****p<0.0001), using GraphPad 755 Prism 7 (SCR 002798).

Cumulative probability distributions were generated in MATLAB (SCR_001622) from 11 sham nRT neurons and 9 TBI neurons, using 200 randomly selected events from each cell.

### Spindle and Epileptic Spike Event Detection in ECoG

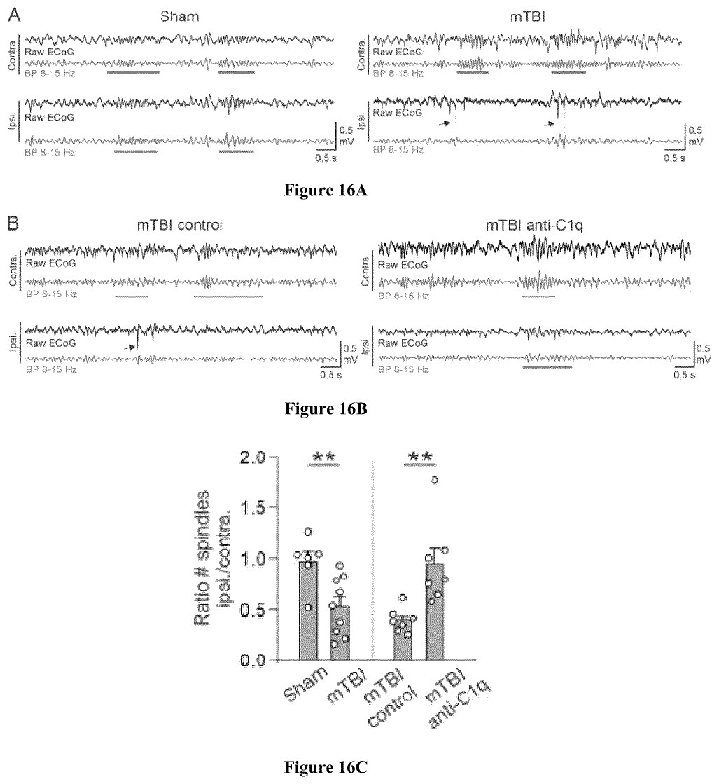

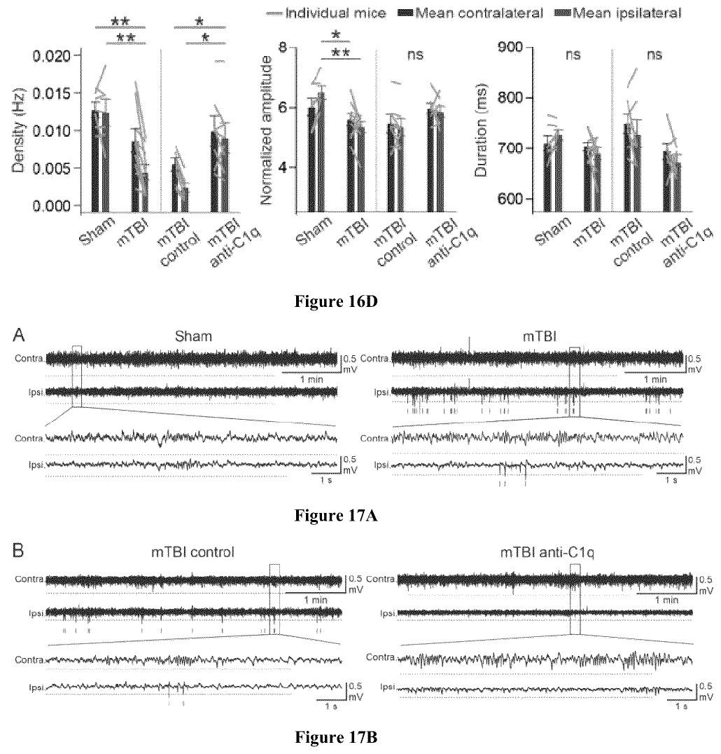

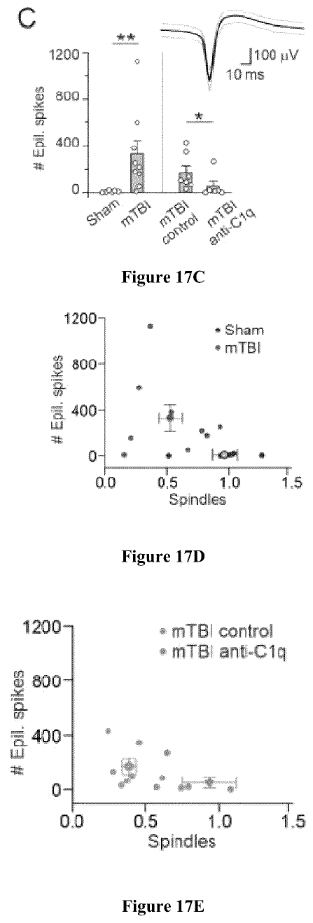

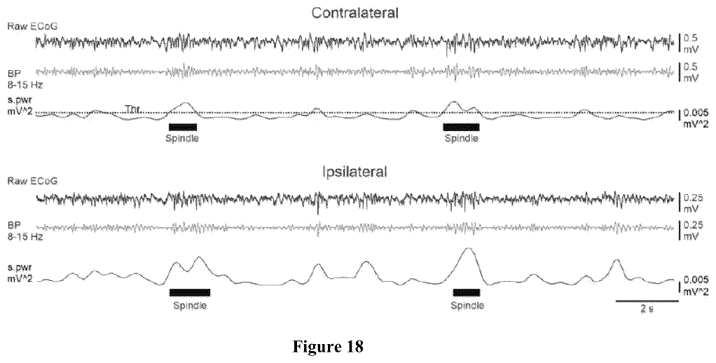

We used the Morlet Wavelet function to detect spindles in the 8-15 Hz frequency range. We applied a threshold of [1.5×the S.D.+1×the mean] of the ECoG power, and detected all events above this threshold that lasted at least 0.5 seconds (FIG. 16, FIG. 18). All detected events were visually validated by a scientist blinded to the groups. The onset and offset times of a spindle event were extended to the closest cycle at 0 crossing before and after the threshold. Amplitudes of spindles were computed from the average amplitude of the spindle (8-15 Hz) power between onset and offset time, divided by RMS of the ECoG signal, and averaged per mouse. Frequency of spindles were determined by extracting the peak frequency from the magnitude of the FFT on each spindle event, followed by calculating the mean intra-frequency per mouse. False positive events that contained epileptic spikes (defined as events that exceeded the threshold of 7×the SD+1×the mean of the baseline, FIGS. 16 and 17) were rejected after visual inspection of a scientist blinded to the groups.

Sleep scoring and data analysis were performed using Spike2 (version 7.20, Cambridge Electronic Design, Cambridge, UK) and Python 3.7 (Python Software Foundation). Epochs of 5 seconds were automatedly scored (Spike 2) and assigned as wakefulness, REM sleep (REM), and NREM/slow wave sleep. Automated scoring was further visually inspected by experienced scientists blinded to the treatment groups. Epochs were assigned as NREM sleep if the ratio of delta (δ, 1.5-4 Hz) to total power (1.5-80 Hz) for ECoG was higher than the threshold value with no locomotor activity. Sleep spindle analysis was performed during NREM sleep for a period of 12 hours (7 am-7 pm) at day 20 or 21 post-mTBI/sham surgery. Epileptic spikes were analyzed during the same time frame. Recordings were not analyzed during locomotion as it was challenging to reliably distinguish movement artifacts from epileptic spikes (FIG. 17).

### Anti-C1q Antibody

The anti-C1q antibody, M1, described herein shows robust binding to mouse C1q and can inhibit functional complement activity in serum from a variety of animal species (See Lansita et al 2017). Previous studies have reported no toxicity in rodents and monkeys (See Lansita et al 2017) and demonstrated in vivo inhibition in several mouse models (See McGonigal et al 2013, Vukojicic et al 2019). Mice were administered i.p. injections of the anti-C1q antibody M1 or a mouse IgG1 isotype control antibody 24 hours after TBI or sham surgery, and continued receiving treatment every three days (four days post-TBI, seven days post-TBI, etc.) for three weeks.

### Treatment Paradigm and Tissue Lysis

For the PK study, mice underwent sham or TBI surgery on Day 0, and were treated intraperitoneally with 100 mg/kg of anti-C1q antibody (M1) or isotype control on Day 1 and 4. Mice were perfused with PBS on Day 5. Plasma and brains (ipsi- and contra-lateral sides) were collected and flash frozen. Brains (without olfactory bulb and cerebellum) were lysed in 1:10 w/v BupH™ Tris Buffered Saline (Thermo Scientific 28379)+protease inhibitor cocktail (Thermo Scientific A32963) by homogenizing with 7 mm steel bead in Qiagen TissueLyser for two minutes at 30 Hz. Lysates were then spun at 17,000×g for 20 minutes. Supernatants were used for ELISA assays.

### Pharmacokinetic (PK) and Pharmacodynamic (PD) ELISA Assays

The levels of free anti-C1q drug M1 (PK), free C1q, total C1q, C1s and albumin were measured using sandwich ELISAs. Black 96 well plates (Nunc 437111) were coated with 75 μL of respective capture protein/antibody: human C1q protein for PK (complement Tech), mouse monoclonal anti-C1q (Abcam, ab71940) for C1q-free, rabbit polyclonal anti-C1q (Dako, A0136) and rabbit polyclonal anti mouse C1s (LSBio, C483829) for C1s, in bicarbonate buffer (pH 9.4) overnight at 4° C. Next day, the plates were washed with dPBS pH 7.4 (Dulbecco's phosphate-buffered saline) and blocked with dPBS containing 3% bovine serum albumin (BSA). Standard curves were prepared with purified proteins in assay buffer (dPBS containing 0.3% BSA and 0.1% Tween20). Samples were prepared in the assay buffer at appropriate dilutions. The blocking buffer was removed from the plate by tapping. Standards and samples were added at 75 μL per well in duplicates and incubated with shaking at 300 rpm at room temperature for one hour for PK measurements. For complement assays, samples were incubated overnight at 4° C. followed by 37° C. for 30 minutes and then room temperature for one hour. Plates were then washed three times with dPBS containing 0.05% Tween20 and 75 μL of alkaline-phosphatase conjugated secondary antibodies (goat anti-mouse IgG for PK, M1 for C1q free, rabbit polyclonal anti-C1q for C1q total, rabbit polyclonal anti-C1s for C1s) were added to all wells. Plates were incubated at room temperature with shaking for one hour, washed three times with dPBS containing 0.05% Tween20 and developed using 75 μL of alkaline phosphatase substrate (Life Technologies, T2214). After 20 minutes at room temperature, plates were read using a luminometer. Albumin assay was done using a matched antibody pair from Abcam (ab210890), followed by Avidin-AP secondary antibody for detection. Standards were fit using a 4PL logistic fit and concentration of unknowns determined. Analyte levels were corrected for dilution factors.

### Example 2: Secondary C1q Expression Coincides with Chronic Inflammation, Neurodegeneration, and Synaptic Dysfunction in the Thalamus

To determine the secondary, long-term effects of mTBI, we induced a mild cortical impact injury to the right primary somatosensory cortex (S1) of adult mice (FIG. 1A), and assessed its impact on the brain three weeks later. This period corresponds to the latent phase in humans, when the brain is undergoing adaptive and maladaptive changes after injury. We determined neuron count and gliotic inflammation in the corticothalamic circuit by immunofluorescent staining of coronal brain sections with markers of neurons (NeuN) and of glial inflammation (C1q, classical complement pathway; GFAP, astrocytes; IBA1, microglia/macrophages) (FIGS. 1C-1E). Three weeks after surgery, mTBI mice had significantly higher GFAP, C1q, and IBA1 expression in the peri-TBI S1 cortex and the functionally connected ventrobasal thalamus (VB) and reticular thalamic nucleus (nRT) than sham mice did (FIGS. 1B-1E). Inflammation of the cortex occurred within 24 hours after injury, while the functionally connected nRT and VB only displayed glial changes around five days later, suggesting that thalamic inflammation is a secondary consequence of cortical injury. We also saw increased expression of similar inflammatory markers in thalamic tissue from human TBI patients, confirming that thalamic inflammation is a consequence of TBI in humans too (FIG. 7).

Glial inflammation was associated with significant neuronal loss in the thalamic region, particularly in the nRT (FIGS. 1D-1E, FIG. 2A), which receives the majority of its glutamatergic inputs from the cortex. The nRT of mTBI mice had significantly fewer neurons than the nRT of sham mice, particularly in the “body” region which receives most of its excitatory inputs from the injured somatosensory cortex (FIGS. 2B-2C). This result suggests that the inflammation follows the long-range, corticothalamic circuit, from the injured cortex to the connected thalamus.

To test whether C1q might mark functional damage in this circuit, we performed whole-cell patch-clamp recordings in the cortex and thalamus of brain slices obtained three to six weeks after injury. We recorded layer 5 pyramidal neurons and fast-spiking GABAergic interneurons in the peri-TBI S1 cortex, glutamatergic neurons in the VB, and GABAergic neurons in the nRT. The neurons' intrinsic membrane electrical properties and the spontaneous excitatory and inhibitory postsynaptic current (sEPSC and sIPSC) properties were similar between sham and mTBI mice in both the peri-TBI cortex and the VB thalamus (see Table 4 for details). However, in the nRT, mTBI led to a reduction in the frequency of sIPSCs (FIGS. 2D-2E). Furthermore, nRT sEPSCs were smaller in amplitude, and trended toward a lower frequency (FIGS. 2F-2G). Immunofluorescence staining for GFP in mice expressing Thy1-GCaMP6f, a marker of neuronal calcium levels in corticothalamic neurons, revealed reduced fluorescence in the thalamus after mTBI (FIGS. 2H-2I), suggesting that the corticothalamic circuit is indeed impaired.

We conclude that the major long-term effect of mTBI on corticothalamic circuits involves disruption of synaptic transmission in the nRT, which coincides with increased C1q expression, reduced cortical inputs, and local neuronal loss. In contrast, neurons in the peri-TBI cortex and the VB appear normal at chronic stages post-mTBI (Table 4), suggesting that inflammation in particular, increased C1q expression in these regions is not associated with long-term dysfunction in neuronal excitability or synaptic function.

TABLE 4
Summary of intrinsic properties, EPSC, and IPSC data recorded from S1 cortex, VB, and nRT. Mice were recorded
between three and six weeks post-TBI, and recording conditions are described in the patch-clamp electrophysiology
section of the methods. A Mann-Whitney test was performed for statistical analysis.

| | | | | | | AP | AP | AP | | | |
|---|---|---|---|---|---|---|---|---|---|---|---|
| Intrinsic | Cm | Vm | Rin | Tau | Rheobase | Thr. | Dur. | Amp. | | | |
| features | (pF) | (mV) | (MOhm) | (ms) | (pA) | (mV) | (ms) | (mV) | cells | slices | mice |
| L5 pyr. | | | | | | | | | | | |
| sham | 94 ± 9.5 | −79 ± 2.2 | 388 ± 22 | 33 ± 2.0 | 55 ± 7.1 | −55 ± 0.7 | 4.2 ± 0.2 | 71 ± 1.2 | 21 | 9 | 6 |
| TBI | 91 ± 9.7 | −71 ± 2.4 | 421 ± 43 | 36 ± 4.8 | 54 ± 9.2 | −51 ± 1.1 | 4.1 ± 0.3 | 60 ± 2.4 | 21 | 10 | 6 |
| MW | ns | 0.03 | ns | ns | ns | 0.01 | ns | 0.0001 | | | |
| p-value | | | | | | | | | | | |
| L5 FS | | | | | | | | | | | |
| sham | 58 ± 6.0 | −70 ± 2.5 | 473 ± 71 | 33 ± 5.2 | 40 ± 8.8 | −54 ± 0.4 | 1.7 ± 0.2 | 58 ± 2.4 | 9 | 6 | 6 |
| TBI | 54 ± 7.3 | −72 ± 2.0 | 573 ± 91 | 27 ± 3.1 | 34 ± 5.7 | −57 ± 1.2 | 1.7 ± 0.2 | 57 ± 2.2 | 14 | 9 | 6 |
| MW | ns | ns | ns | ns | ns | 0.03 | ns | ns | | | |
| p-value | | | | | | | | | | | |
| VB | | | | | | | | | | | |
| sham | 166 ± 18 | −62 ± 1.2 | 198 ± 32 | 28 ± 3.2 | 158 ± 17 | −50 ± 0.9 | 2.7 ± 0.2 | 51 ± 2.9 | 21-22 | 7 | 6 |
| TBI | 169 ± 12 | −65 ± 1.2 | 200 ± 19 | 27 ± 2.6 | 170 ± 13 | −51 ± 0.9 | 2.3 ± 0.1 | 49 ± 2.6 | 28-33 | 8 | 8 |
| MW | ns | ns | ns | ns | ns | ns | ns | ns | | | |
| p-value | | | | | | | | | | | |
| nRT | | | | | | | | | | | |
| sham | 91 ± 13 | −74 ± 3.1 | 402 ± 58 | 27 ± 2.8 | 42 ± 5.5 | −53 ± 1.3 | 1.3 ± 0.1 | 55 ± 2.2 | 10 | 7 | 5 |
| TBI | 82 ± 10 | −60 ± 3.5 | 523 ± 114 | 44 ± 11 | 69 ± 26 | −50 ± 1.5 | 1.4 ± 0.1 | 45 ± 3.2 | 9 | 6 | 5 |
| MW | ns | 0.009 | ns | ns | ns | ns | ns | 0.03 | | | |
| p-value | | | | | | | | | | | |

| | | | | Half- | Rise | Decay | | | | |
|---|---|---|---|---|---|---|---|---|---|---|
| | Frequency | Charge | Amplitude | width | time | time | Tau | | | |
| EPSCs | (Hz) | (pA × ms) | (pA) | (ms) | (ms) | (ms) | (ms) | cells | slices | mice |
| L5 pyr. | | | | | | | | | | |
| sham | 0.2 ± 0.1 | 117 ± 7.5 | 25 ± 1.3 | 2.2 ± 0.2 | 1.1 ± 0.1 | 5.2 ± 0.4 | 4.0 ± 0.2 | 21 | 9 | 6 |
| TBI | 0.6 ± 0.2 | 107 ± 9.1 | 27 ± 2.4 | 2.1 ± 0.2 | 0.9 ± 0.1 | 5.5 ± 0.4 | 3.0 ± 0.2 | 16 | 10 | 6 |
| MW | ns | ns | ns | ns | ns | ns | 0.01 | | | |
| p-value | | | | | | | | | | |
| L5 FS | | | | | | | | | | |
| sham | 1.4 ± 0.5 | 56 ± 2.4 | 26 ± 0.8 | 1.0 ± 0.1 | 0.4 ± 0.02 | 3.0 ± 0.4 | 1.6 ± 0.1 | 8 | 6 | 6 |
| TBI | 1.2 ± 0.4 | 55 ± 6.3 | 29 ± 3.6 | 1.1 ± 0.2 | 0.4 ± 0.04 | 3.0 ± 0.6 | 1.5 ± 0.2 | 11 | 10 | 6 |
| MW | ns | ns | ns | ns | ns | ns | ns | | | |
| p-value | | | | | | | | | | |
| VB | | | | | | | | | | |
| sham | 0.9 ± 0.2 | 54 ± 4.4 | 22 ± 1.6 | 1.4 ± 0.2 | 0.4 ± 0.03 | 4.2 ± 0.7 | 1.9 ± 0.2 | 15 | 7 | 7 |
| TBI | 1.0 ± 0.4 | 50 ± 4.6 | 21 ± 1.6 | 1.2 ± 0.1 | 0.5 ± 0.1 | 3.6 ± 0.5 | 1.8 ± 0.2 | 13 | 8 | 7 |
| MW | ns | ns | ns | ns | ns | ns | ns | | | |
| p-value | | | | | | | | | | |
| nRT | | | | | | | | | | |
| sham | 2.9 ± 0.6 | 32 ± 2.3 | 28 ± 1.8 | 0.7 ± 0.1 | 0.3 ± 0.03 | 1.7 ± 0.2 | 0.7 ± 0.1 | 11 | 6 | 6 |
| TBI | 1.9 ± 0.4 | 30 ± 2.0 | 22 ± 1.8 | 0.9 ± 0.1 | 0.3 ± 0.1 | 2.3 ± 0.3 | 1.0 ± 0.2 | 9 | 7 | 7 |
| MW | ns | ns | 0.04 | ns | ns | ns | ns | | | |
| p-value | | | | | | | | | | |

| | | | | Half− | Rise | Decay | | | | |
|---|---|---|---|---|---|---|---|---|---|---|
| | Frequency | Charge | Amplitude | width | time | time | Tau | | | |
| IPSCs | (Hz) | (pA × ms) | (pA) | (ms) | (ms) | (ms) | (ms) | cells | slices | mice |
| L5 pyr. | | | | | | | | | | |
| sham | 1.2 ± 0.2 | 471 ± 49 | 39 ± 2.7 | 6.1 ± 0.3 | 1.1 ± 0.1 | 21.1 ± 1.0 | 10.0 ± 0.4 | 19 | 8 | 6 |
| TBI | 1.3 ± 0.3 | 397 ± 36 | 36 ± 2.7 | 5.6 ± 0.3 | 1.2 ± 0.1 | 18.0 ± 1.2 | 8.4 ± 0.4 | 16 | 6 | 6 |
| MW | ns | ns | ns | ns | ns | 0.04 | 0.02 | | | |
| p-value | | | | | | | | | | |
| VB | | | | | | | | | | |
| sham | 2.4 ± 0.8 | 884 ± 360 | 47 ± 5.6 | 5.4 ± 0.4 | 1.2 ± 0.1 | 20 ± 3.8 | 12 ± 4.7 | 10 | 6 | 5 |
| TBI | 1.9 ± 0.4 | 566 ± 127 | 42 ± 5.1 | 6.6 ± 0.8 | 1.1 ± 0.1 | 24 ± 6.6 | 10 ± 2.9 | 11 | 5 | 4 |
| MW | ns | ns | ns | ns | ns | ns | ns | | | |
| p-value | | | | | | | | | | |
| nRT | | | | | | | | | | |
| sham | 0.9 ± 0.2 | 836 ± 103 | 21 ± 2.7 | 16 ± 2.1 | 1.3 ± 0.1 | 69 ± 9.0 | 55 ± 6.0 | 13 | 5 | 4 |
| TBI | 0.6 ± 0.2 | 1144 ± 141 | 18 ± 1.6 | 30 ± 2.5 | 2.7 ± 0.4 | 79 ± 7.9 | 73 ± 3.5 | 22 | 9 | 6 |
| MW | 0.02 | ns | ns | 0.0003 | 0.0002 | ns | 0.04 | | | |
| p-value | | | | | | | | | | |

### Example 3: Chronic Increase in C1q is Mediated by Microglia in the Thalamus

To determine the cellular origin of C1q in the thalamus, we microdissected nRT and VB tissue three weeks post injury (FIG. 3A), and performed single-nucleus RNA sequencing (snRNA-seq) on 6,228 nuclei from sham mice and 5,220 nuclei from mTBI mice, allowing us to robustly capture neuronal and glial populations from the same preparation without isolation artifacts. After correcting for ambient RNA and removing potential doublets, clustering analysis identified the expected cell types, including microglia (Cx3cr1, P2ry12), astrocytes (CIdn10, Fgfr3), oligodendrocytes (Mobp, Olig1), oligodendrocyte progenitors (Sox8, Pdgfra), GABAergic neurons (Gad1, Gad2), and glutamatergic neurons (Slc17a6, Slc17a7), which originated from adjacent thalamocortical relay nuclei (FIG. 3B, FIG. 8A). The cellular composition was similar between sham and mTBI samples (FIGS. 8B-8C).

Microglia expressed high levels of C1qa, C1qb, and C1qc, the three genes that together encode the 18 subunits of C1q (FIG. 3C, FIG. 9B). However, their expression within the nuclear RNA was not significantly different between mTBI and sham samples (FIG. 3D, Figure S3B), consistent with previous reports on the encoding of microglia activation in cytoplasmic RNA. Mature oligodendrocytes and astrocytes in both sham and TBI mice expressed C4b, which acts downstream of C1q in the classical complement pathway (FIG. 3E). C4b expression in nuclear RNA increased 5.2-fold in one subcluster of oligodendrocytes after mTBI (FIG. 3F, FIGS. 9C-9F), but did not significantly increase in astrocytes. Transcripts for other components of the complement pathway, such as C2 and Hc, were not detected (FIGS. 9B, 9H).

These observations made microglia the likely source of C1q protein, but did not explain the surge of C1q in the thalamus after mTBI. To address this discrepancy, we examined C1qa mRNA in the bulk cytoplasmic fractions of our nuclei preparations using qRT-PCR. This analysis showed a significant increase in C1qa mRNA expression after mTBI, in both the thalamus and the cortex (FIG. 3G). Similarly, C4b expression was upregulated in mTBI mice in these two regions, but expression of other complement molecules such as C3 or He (C5) was not (FIG. 3H, FIG. 9H).

Altogether, our results suggest that microglia are responsible for the increased levels of chronic C1q in mTBI mice, and that C1q likely activates C4b-expressing oligodendrocytes and astrocytes. Consistent with these observations, we only detected a small number of markers of microglial (Apoe, Cst3) and astrocytic (Apoe, C1u) activation after TBI (FIG. 9A).

### Example 4: mTBI Leads to Selective Changes in Mitochondrial Gene Expression in the nRT

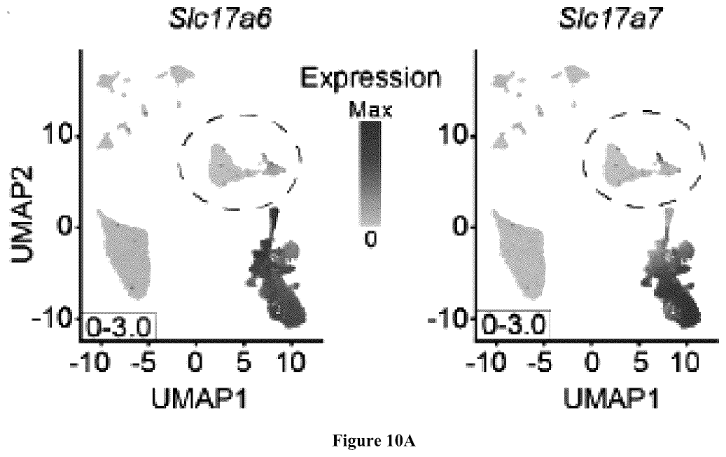

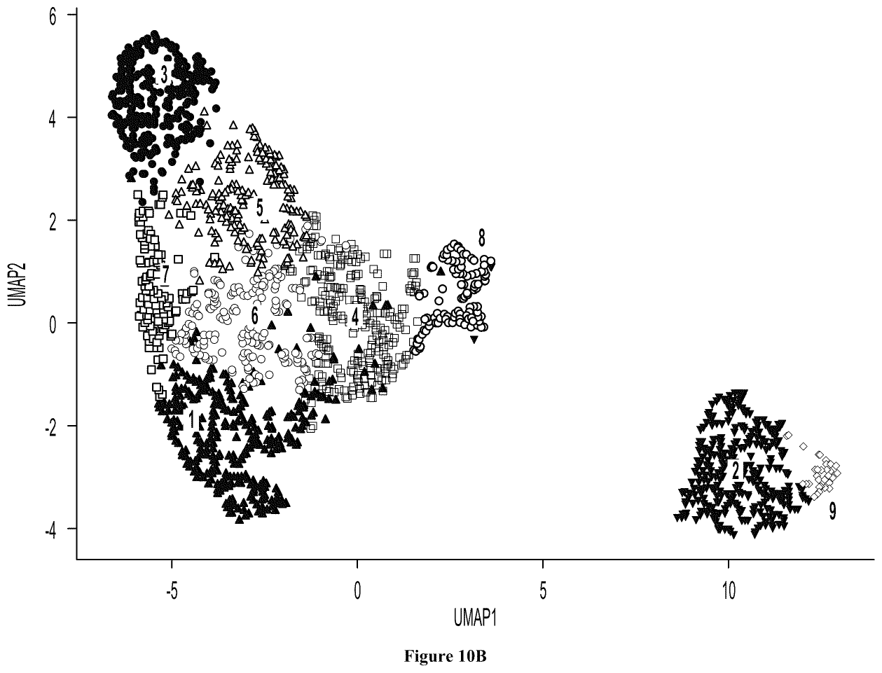

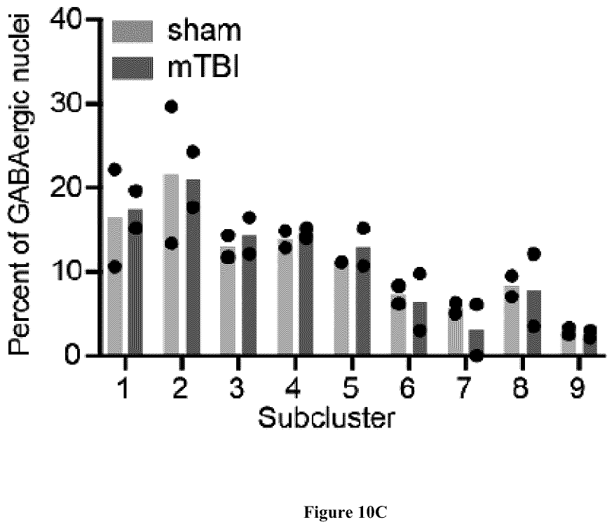

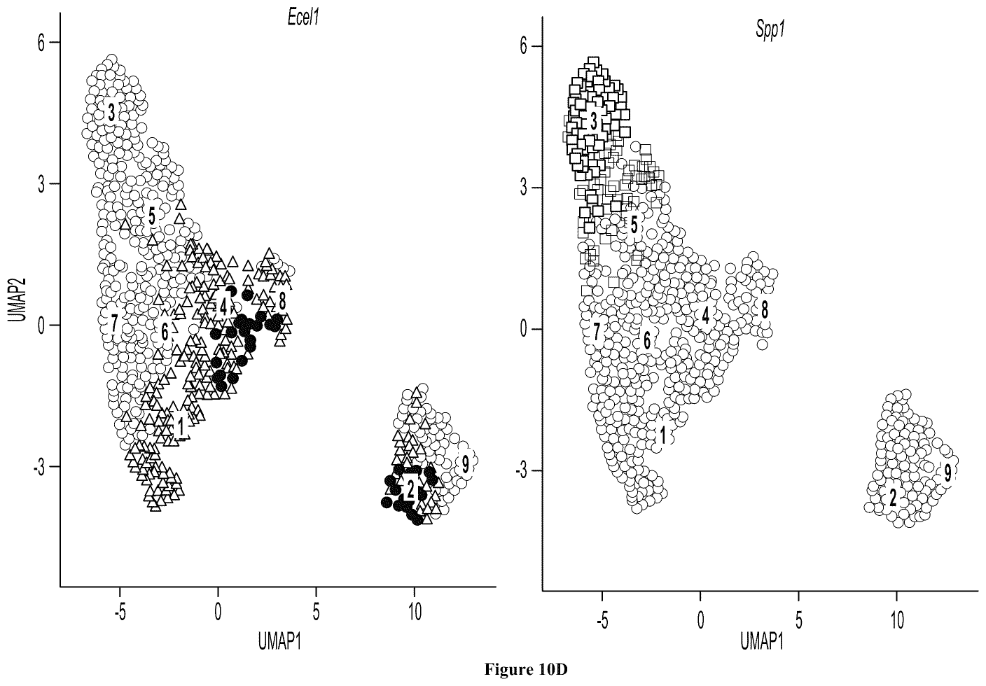

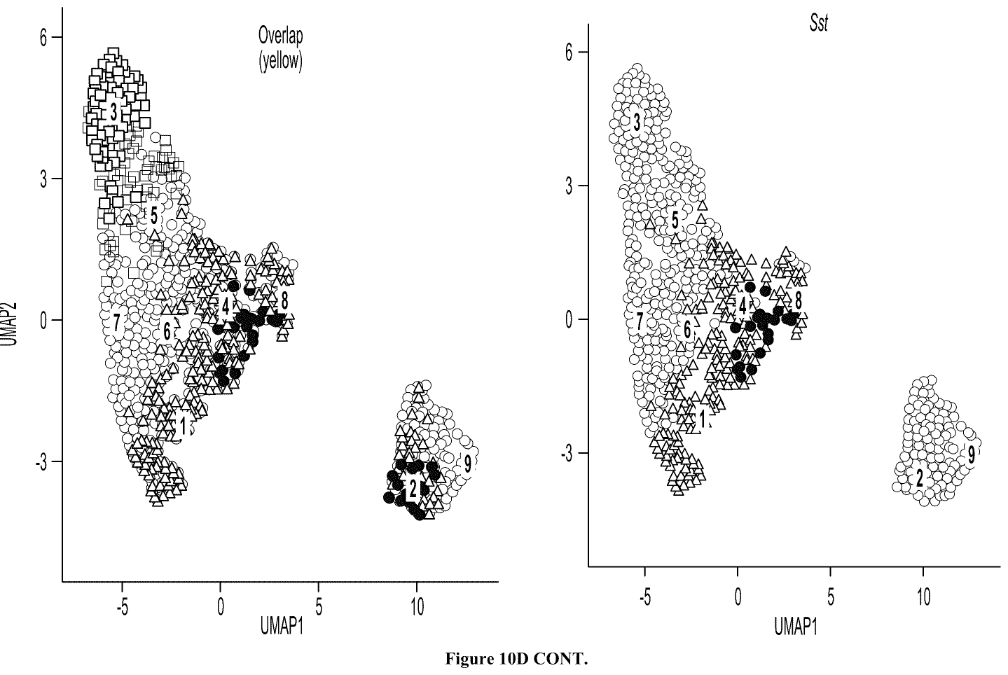

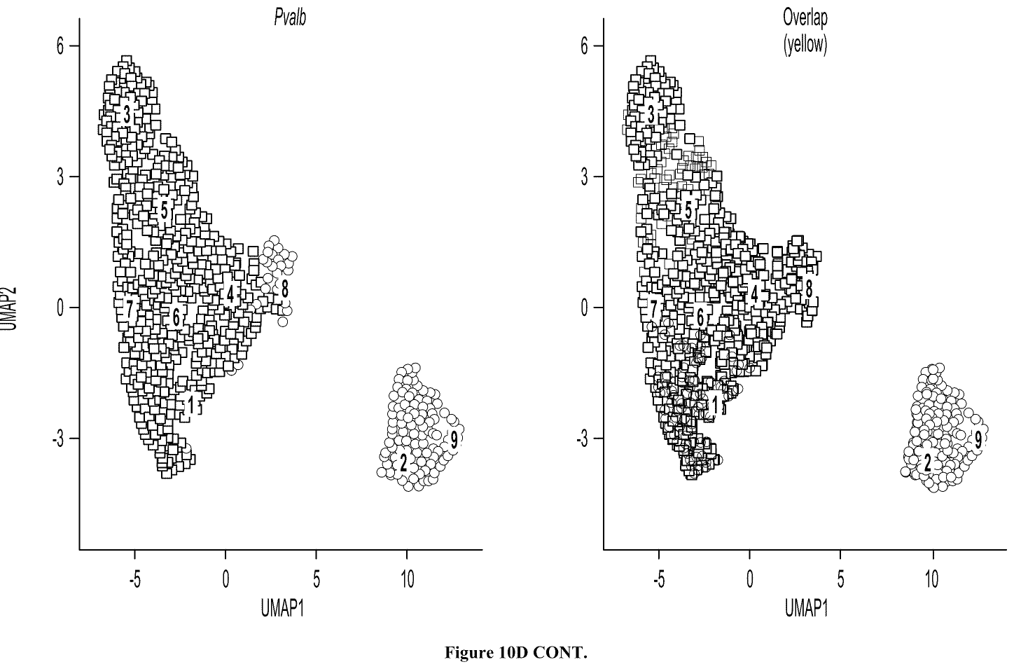

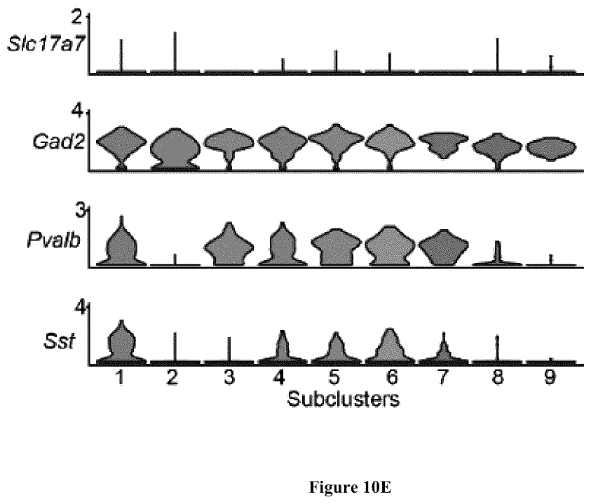

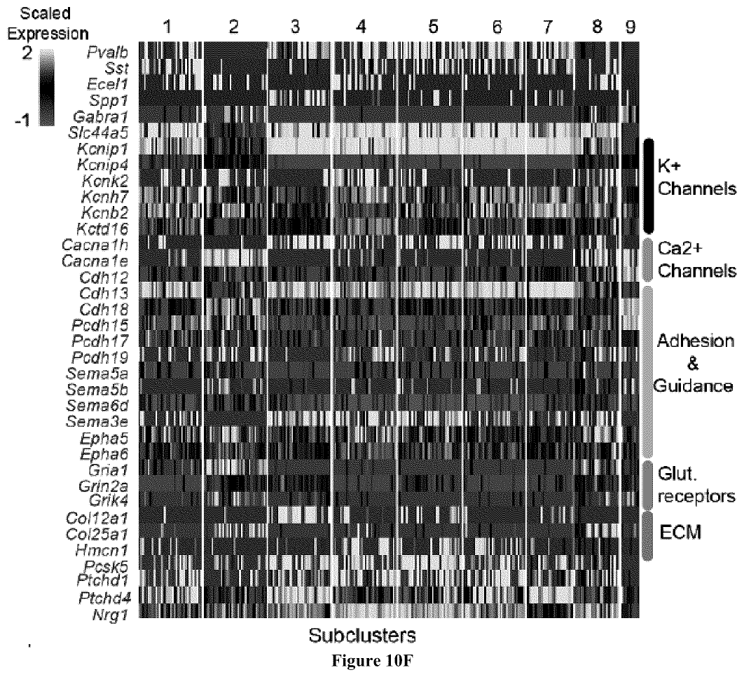

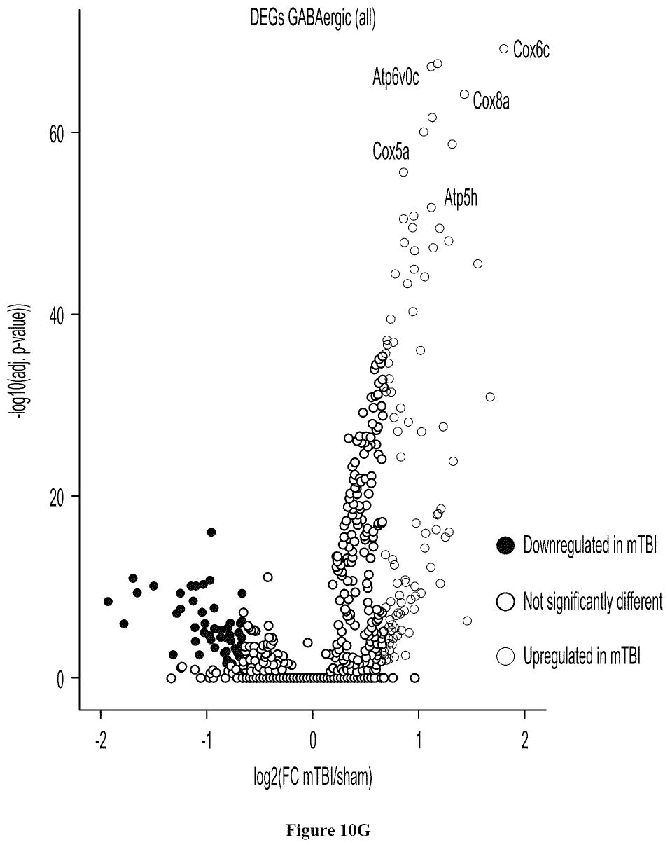

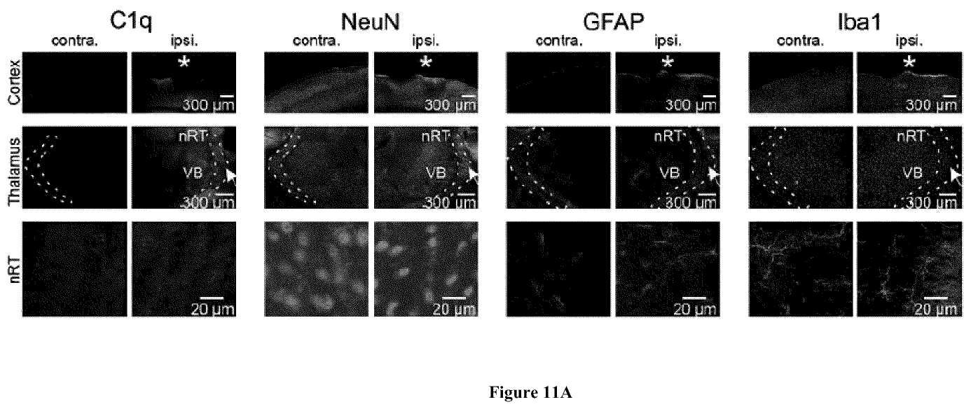

Since we had detected synaptic anomalies and neuronal loss in the nRT after mTBI, we also investigated potential changes in gene expression in nRT GABAergic neurons. Clustering of the GABAergic neurons (Slc17a7 and Slc17a6 negative, Gad2 positive, FIG. 10A) revealed nine subclusters (FIG. 10B) that were characterized by expression of genes previously reported in the nRT (Pvalb, Spp1 and Ecel1, Cacna1h and Cacna1e). We also observed three clusters that were Pvalb negative (subclusters 2, 8 and 9, FIGS. 10D-10F), which would have been missed in previous studies using a Pvalb reporter mouse for cell selection. Notably, the relative size of the subclusters did not change between mTBI and sham mice (FIG. 10C), suggesting a lack of selective vulnerability within the nRT.

Components of the complement pathway were not differentially expressed between mTBI and sham in any of the GABAergic subclusters. In contrast, several genes related to mitochondrial function and oxidative phosphorylation, including Cox6c and Cox5a, were upregulated in all GABAergic neurons after mTBI (FIG. 10G).

Overall, these data support the existence of multiple subclusters of nRT GABAergic neurons that differ in their expression of key marker genes such as Pvalb, Spp1, and Ecel1. These observations confirm that the source of thalamic C1q after mTBI is not neurons. They also suggest mitochondria as potential mediators of neuronal loss or synaptic dysfunction in GABAergic nRT neurons after mTBI.

### Example 5: Blocking C1q Function Reduces Chronic Glial Inflammation and Neuron Loss

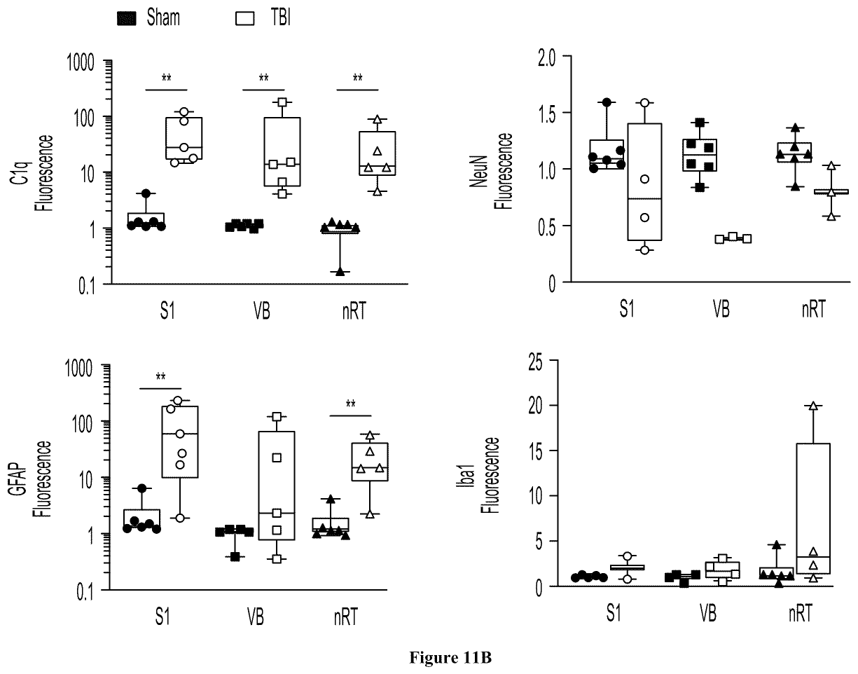

Increased C1q expression was chronic (FIGS. 11A-11B) and might therefore explain the long-term effects of mTBI. To test this hypothesis, we used an antibody that specifically binds to C1q and blocks its downstream activity. Mice were given i.p. injections of the C1q antibody or a mouse IgG1 isotype control 24 hours after mTBI or sham surgery, followed by twice-weekly treatments for three weeks.

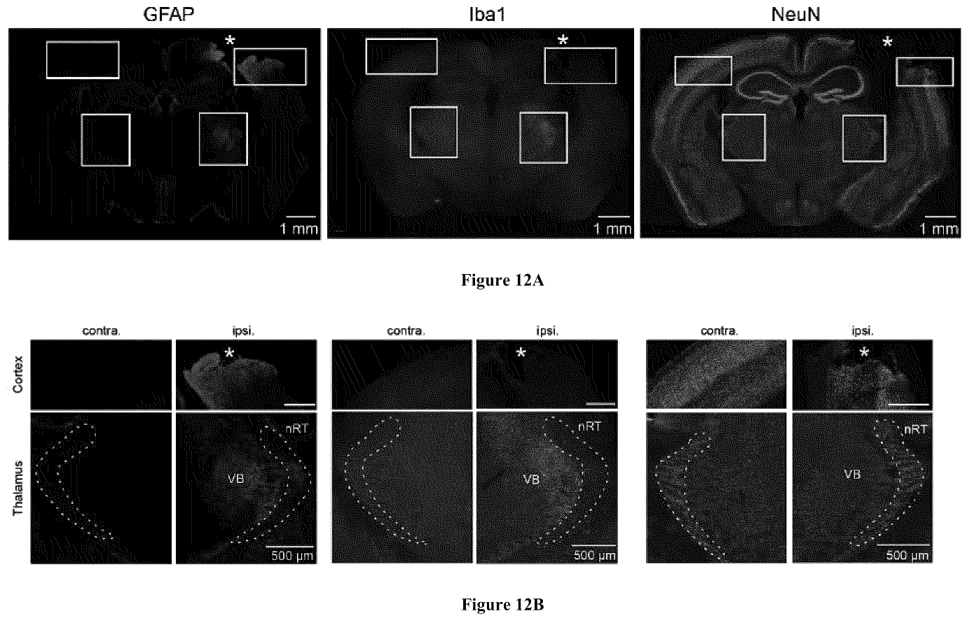

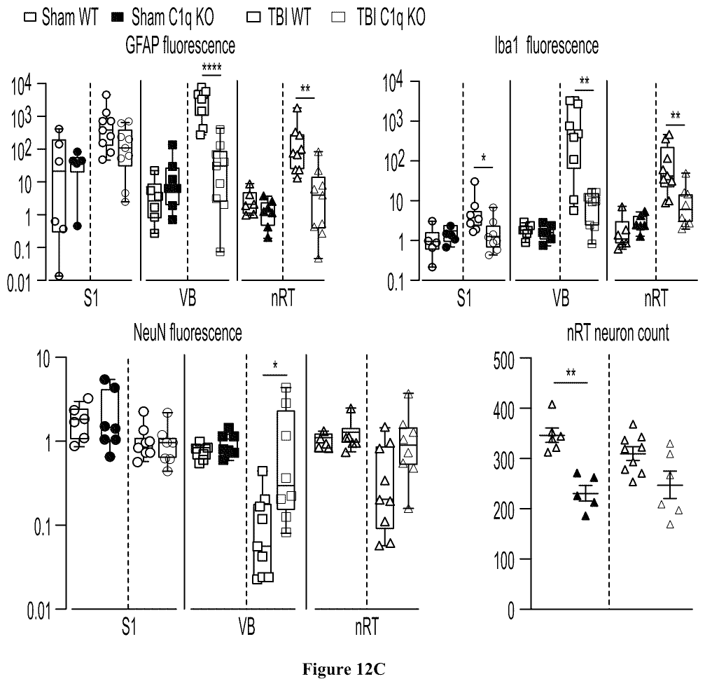

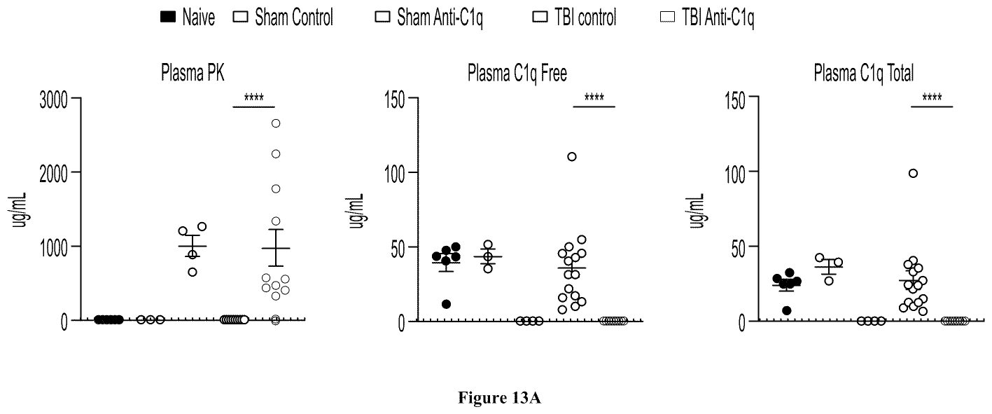

mTBI mice treated with the anti-C1q antibody showed a strong reduction in inflammation and reduced neuronal loss (FIG. 4A-C) relative to control-treated mTBI mice, as monitored by immunofluorescent staining, and on average had the same number of nRT neurons as antibody-treated sham mice (FIG. 4C). mTBI mice treated with the control IgG still showed inflammation and neuron loss three weeks after mTBI (FIG. 4). As an alternative approach to the antibody treatment, we repeated the study using C1q−/− mice and found that they too exhibited reduced chronic inflammation and reduced neuron loss in the nRT after TBI (FIG. 12).

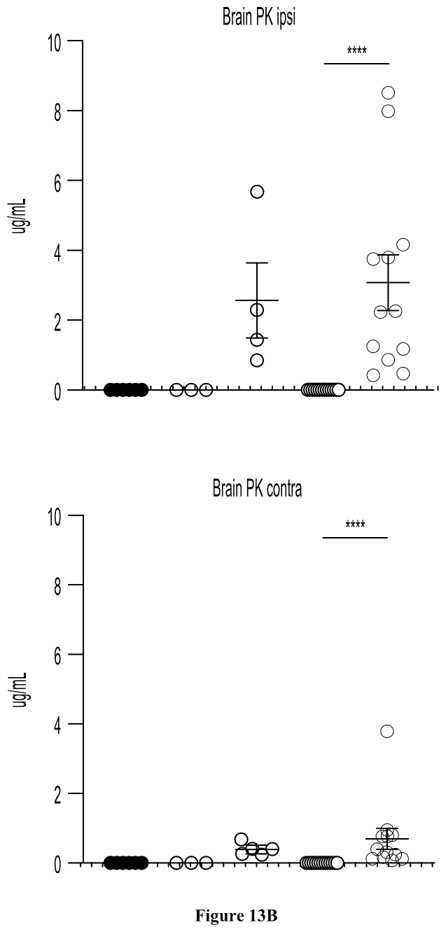

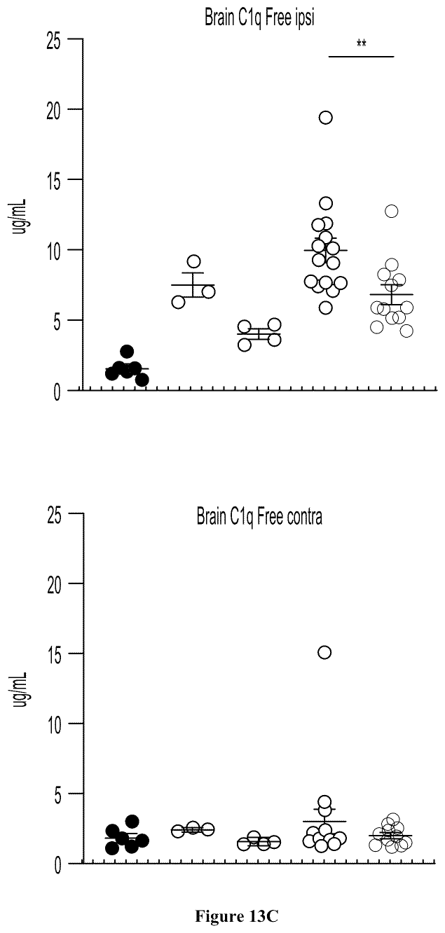

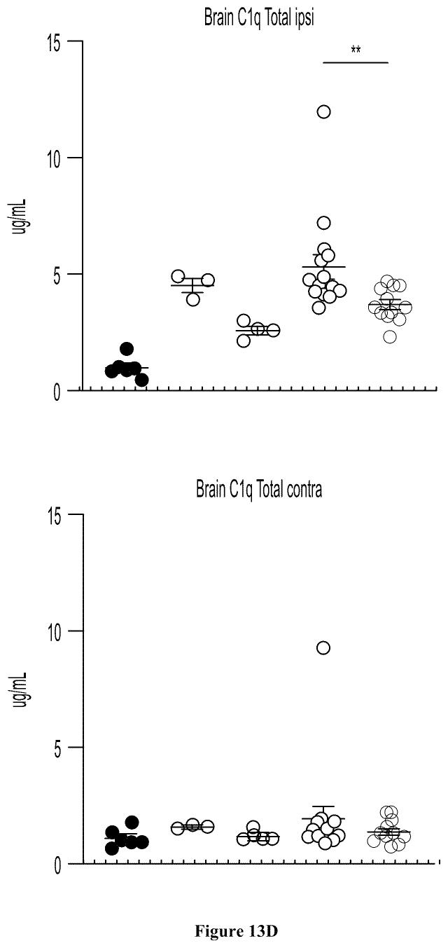

To confirm that the anti-C1q antibody exerted its effect in the brain rather than peripherally, we measured its concentration and that of C1q in the brain and in the plasma (FIG. 13). Free anti-C1q antibody was detected in the brain of antibody-treated sham and mTBI mice, at a slightly greater concentration in the ipsilateral side (0.4-8.6 ug/ml) than the contralateral side (0.09-3.8 ug/m). This was accompanied by a lower concentration of C1q than in sham or mTBI mice that had not received the anti-C1q antibody, most significantly on the ipsilateral side (FIGS. 13C-13D). These observations strongly suggest that the anti-C1q antibody prevents C1q from accumulating after mTBI. The plasma of sham and mTBI mice that had received the antibody had no detectable amount of C1q protein, either free or antibody-bound, indicating that free C1q is fully cleared from the circulation.

These outcomes indicate that C1q may lead to inflammation and neuron loss in mTBI, and that blocking C1q accumulation in the brain reduces these deleterious effects.

### Example 6: TBI Leads to Long-Term Changes in Cortical States and Excitability in Freely Behaving Mice

We next investigated the longitudinal impact of mTBI, using brain rhythms as a readout of corticothalamic circuit function in vivo. To this end, we implanted chronic wireless electrocorticographic (ECoG) devices into sham and mTBI mice during the craniotomy/mTBI induction surgery, returned mice to their home cages for chronic recording, and analyzed changes in ECoG power at 1, 3 and 11 weeks post mTBI (FIG. 5). We observed a chronic increase in broadband power in mTBI during both light epochs (FIGS. 5C-5H) and dark epochs.

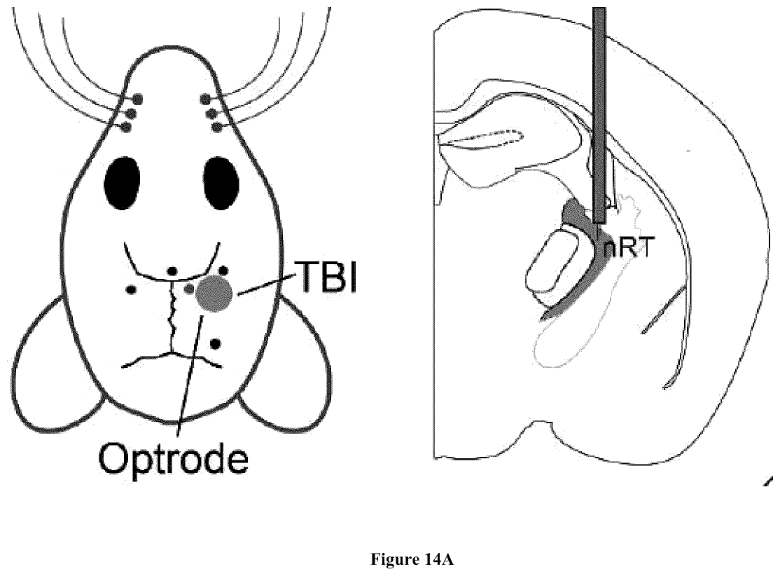

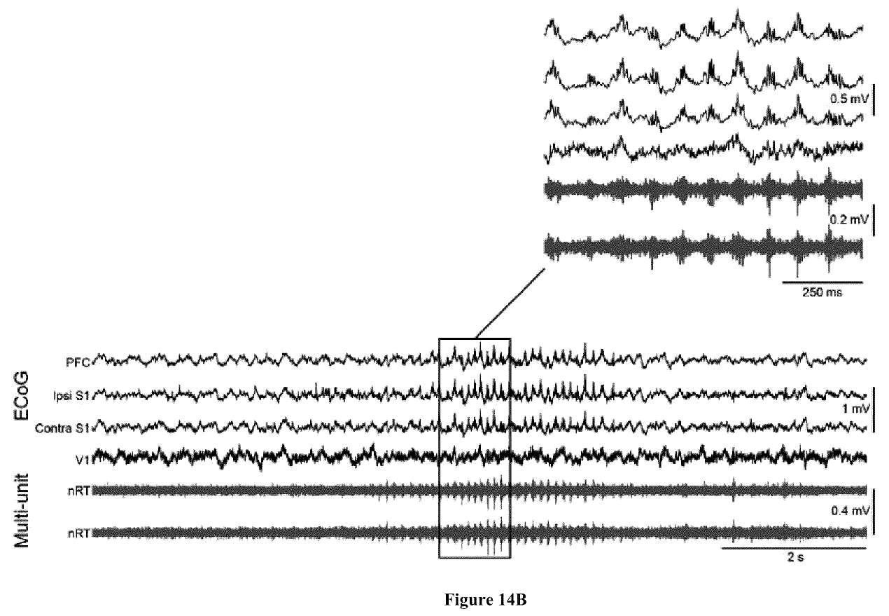

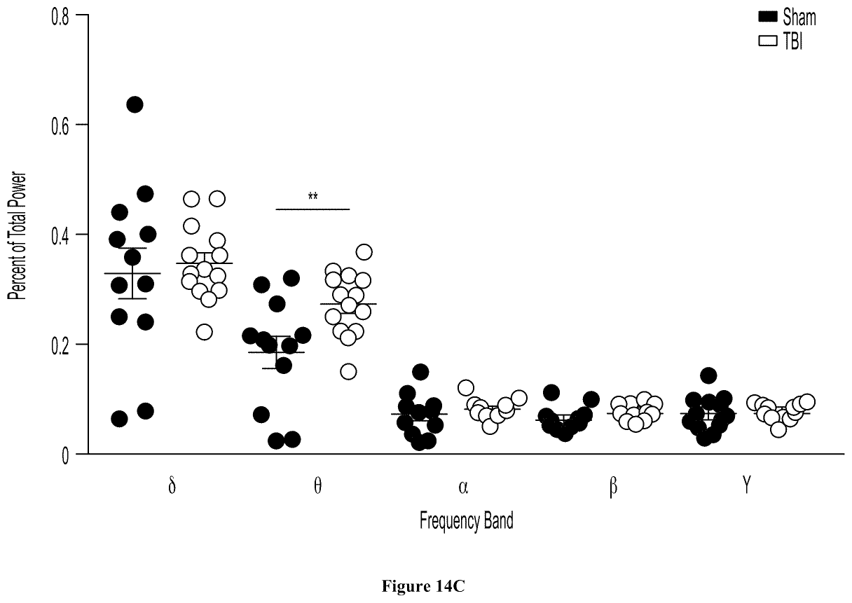

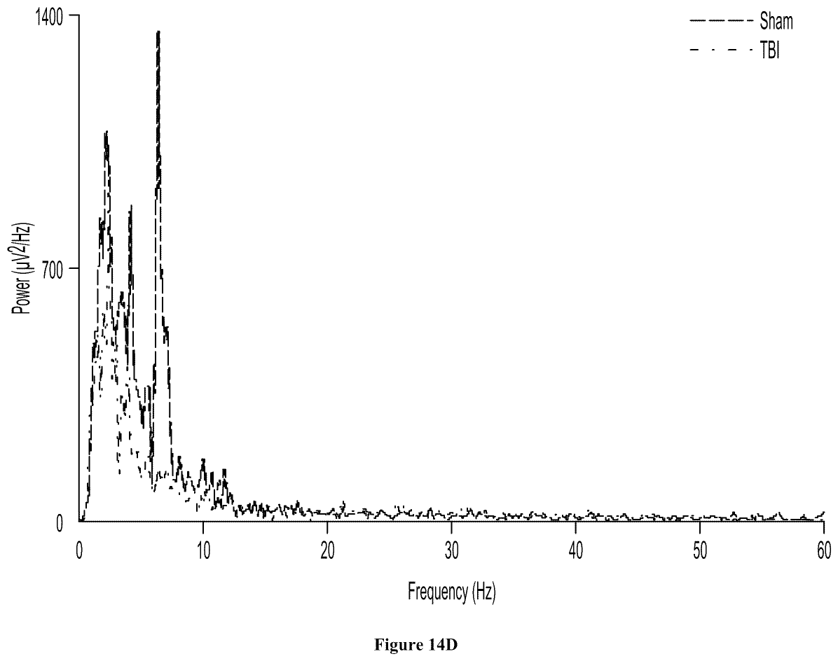

Severe TBI has been shown to lead to epileptogenesis over time, and we investigated whether it might be true of mTBI too. We quantified different types of epileptic activities including epileptiform spikes, epileptic discharges, spike-and-wave discharges, and spontaneous focal or generalized seizures at 24 hours and three weeks after mTBI using previously reported classification. In the first 24 hours, 3 out of 16 mTBI mice, but none of the 8 sham mice, showed generalized tonic-clonic seizures (GTCSs, Table 5). None of the mice showed GTCSs at later time points (up to three weeks) (Table 5). However, at three weeks post-mTBI, we saw more epileptiform spikes in mTBI mice (n=9) than in sham mice (n=5), suggesting an increase in excitability (Table 5). Similarly, in another recording setup using simultaneous ECoG and multi-unit thalamic recordings, mTBI mice had spontaneous epileptiform events that included synchronized thalamic bursting and increased normalized theta power, as early as one week and up to three weeks post-mTBI (FIG. 14).

We conclude that mTBI does alter cortical ECoG activity, by increasing the likelihood of early seizures and the broadband ECoG power at chronic time points.

TABLE 5
Summary of epileptiform activity analysis in sham, TBI, control-treated TBI, and antibody-
treated TBI mice. Mice were recorded continuously starting the day of the TBI up until several
weeks post-TBI. Surgical and recording conditions are described in the methods section
titled “Surgical implantation of devices for chronic ECoG recordings”. Analysis was
performed on the first 24 hours post-TBI, and across a 48 hour window at three weeks post-
TBI. A repeated measures mixed-effects ANOVA was performed for statistical analysis.

| | | | | | Spike-and- | | Generalized | | Acute | |
|---|---|---|---|---|---|---|---|---|---|---|
| First 24 | Epileptiform | | Epileptiform | | wave | | tonic-clonic | | post-injury | |
| hours | spikes | | discharges | | discharges | | seizures | | mortality | |
| Sham | 8/8 | (100%) | 6/8 | (75%) | 0/8 | (0%) | 0/8 | (0%) | 0/8 | (0%) |
| TBI | 16/16 | (100%) | 13/16 | (81%) | 4/16 | (25%) | 3/16 | (19%) | 0/16 | (0%) |
| Drug study | | | | | | | | | | |
| TBI Vehicle | 7/7 | (100%) | 5/7 | (71%) | 0/7 | (0%) | 2/7 | (28%) | 0/7 | (0%) |
| TBI anti-C1q | 7/7 | (100%) | 5/7 | (71%) | 0/7 | (0%) | 1/7 | (14%) | 0/7 | (0%) |

| | | | | | Spike-and- | | Generalized | |
|---|---|---|---|---|---|---|---|---|
| | Epileptiform | | Epileptiform | | wave | | tonic-clonic | |
| 3 weeks | spikes | | discharges | | discharges | | seizures | |
| Sham | 7/7 | (100%) | 1/7 | (14%) | 0/7 | (0%) | 0/7 | (0%) |
| TB | 11/11 | (100%) | 3/11 | (27%) | 1/11 | (9%) | 0/11 | (0%) |
| Drug study | | | | | | | | |
| TBI Vehicle | 7/7 | (100%) | 2/7 | (28%) | 0/7 | (0%) | 0/7 | (0%) |
| TBI anti-C1q | 7/7 | (100%) | 3/7 | (43%) | 0/7 | (0%) | 0/7 | (0%) |

| | | | Spike-and- | Generalized | |
|---|---|---|---|---|---|
| | Epileptiform | Epileptiform | wave | tonic-clonic | |
| | spikes | discharges | discharges | seizures | mice |
| Sham - 24 h | 234 ± 62 | 4 ± 2 | 0 | 0 | 8 |
| TBI - 24 h | 452 ± 178 | 8 ± 6 | 1 ± 0.6 | 0.4 ± 0.2 | 16 |
| Sham - 3 wk | 66 ± 38 | 0.7 ± 0.7 | 0 | 0 | 7 |
| TBI - 3 wk | 292 ± 114 | 2 ± 1 | 0.09 ± 0.09 | 0 | 11 |
| Mixed-effects | ns | ns | ns | ns | |
| analysis | | | | | |
| Drug study | | | | | |
| TBI Vehicle - | 278 ± 79 | 4 ± 1 | 0 | 0.6 ± 0.4 | 7 |
| 24 h | | | | | |
| TBI anti-C1q - | 137 ± 55 | 1 ± 0.5 | 0 | 0.3 ± 0.3 | 7 |
| 24 h | | | | | |
| TBI Vehicle - | 300 ± 92 | 0.3 ± 0.2 | 0 | 0 | 7 |
| 3 wk | | | | | |
| TBI anti-C1q - | 274 ± 50 | 1 ± 0.8 | 0 | 0 | 7 |
| 3 wk | | | | | |
| Mixed-effects | ns | ns | ns | ns | |
| analysis | | | | | |

### Example 7: Anti-C1q Antibody Prevents Changes in Chronic Cortical States in Mice with TBI

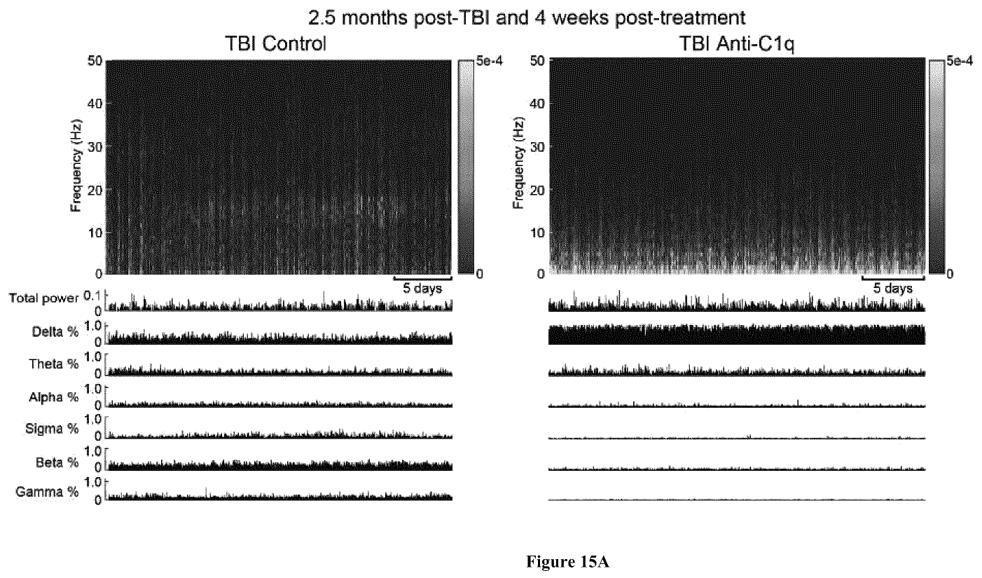

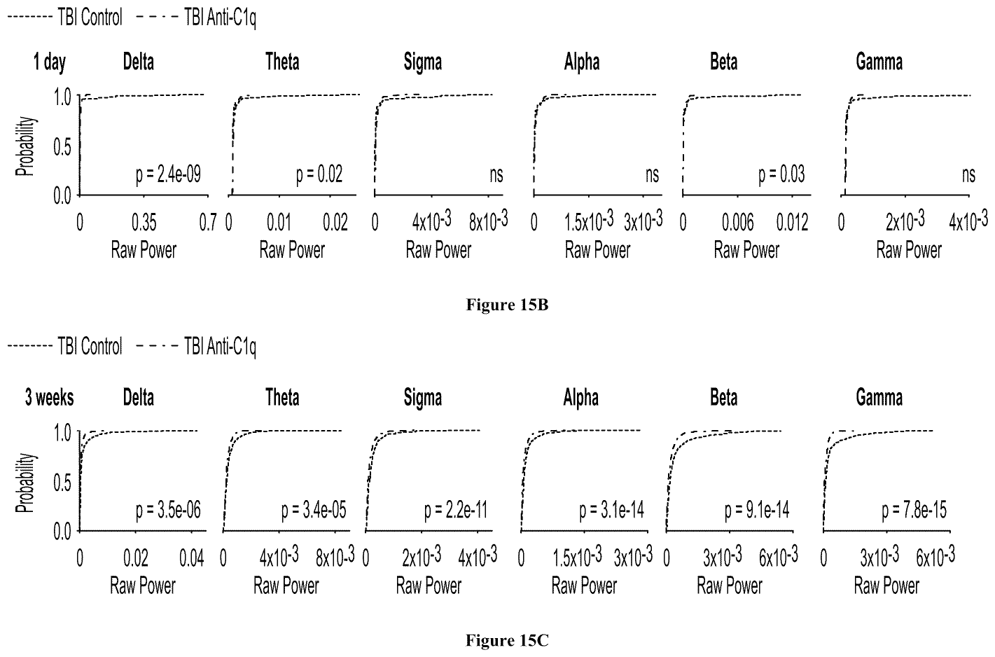

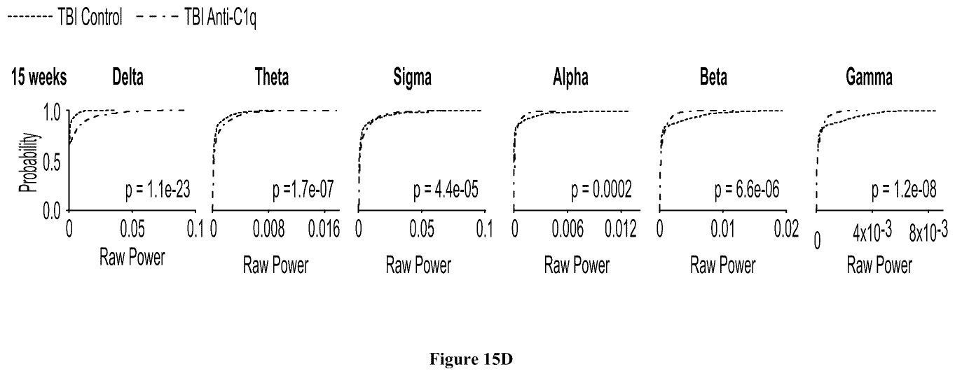

To determine whether blocking C1q could rescue changes in cortical states, we treated mice with the anti-C1q antibody or isotype control for five weeks, starting 24 hours post mTBI, while maintaining ECoG recordings for up to 9-15 weeks post-mTBI (FIG. 6A, FIG. 15). The ECoG spectral features were similar within the first week of anti-C1q antibody or control treatment (FIGS. 6B-6C, FIG. 15B). At three weeks, the anti-C1q group trended toward reduced power across most frequency bands (FIGS. 6D-6E, FIG. 15C), but the reduction was not statistically significant.

Notably, epileptiform activities were not affected by the anti-C1q antibody (Table 5). Three weeks post-mTBI, we saw no GTCSs and no differences in the frequency of epileptic events between control-treated and antibody-treated mTBI mice (Table 5).

These results taken together suggest that blocking C1q after TBI insult protects against secondary changes in cortical states in mice.

### Example 8: mTBI Leads to Loss of Sleep Spindles and Increased Epileptic Spikes which are Prevented by Anti-C1q Treatment

We next investigated the impact of mTBI in vivo using brain rhythms as a readout of corticothalamic circuit function. To this end, we implanted chronic wireless electrocorticographic (ECoG) devices into sham and mTBI mice during the craniotomy/mTBI induction surgery, returned mice to their home cages for chronic recording, and analyzed changes in ECoG rhythms within a 12 hour window three weeks post-surgery (FIG. 16). Given that the nRT is a source for sensory cortex-specific sleep spindles during non-rapid-eye-movement sleep (NREMS) in mice, we focused our analysis on sleep spindles. Three weeks post-surgery, sham mice had similar numbers of sleep spindles in the left and right sensory cortices, but in mTBI mice the cortex ipsilateral to injury showed fewer sleep spindles than the contralateral cortex (FIGS. 16A-16D). mTBI mice also had focal epileptic spikes ipsilateral to the injury (FIGS. 16A-16D). Next, to determine whether blocking C1q could prevent these changes, we treated mice with the anti-C1q antibody or isotype control starting 24 hours post mTBI and analyzed the ECoG three weeks post-mTBI. Mice treated with the anti-C1q antibody showed normal numbers of sleep spindles (FIGS. 16B, 16D, 16E), and less epileptic spikes than the mice treated with the isotype control (FIGS. 17B-17F). These results show that mTBI leads to loss of sleep spindles in the peri-mTBI cortex and causes epileptic spikes, and that blocking the C1q-mediated pathway after mTBI prevents both of these outcomes.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

1. A method of preventing, reducing risk of developing, or treating epilepsy, comprising administering to a subject an inhibitor of the classical complement pathway.

2. The method of claim 1, wherein the epilepsy is an idiopathic generalized epilepsy, idiopathic partial epilepsy, symptomatic generalized epilepsy or symptomatic partial epilepsy.

3. The method claim 1, wherein the symptomatic partial epilepsy is temporal lobe epilepsy.

4-8. (canceled)

9. The method of claim 1, wherein the inhibitor is administered to a patient suffering from a traumatic brain injury, hypoxic brain injury, brain infection, stroke, or genetic syndrome.

10-17. (canceled)

18. The method of claim 1, wherein the inhibitor of the classical complement pathway is a C1q inhibitor.

19. The method of claim 18, wherein the C1q inhibitor is an antibody, an aptamer, an antisense nucleic acid or a gene editing agent.

20. The method of claim 19, wherein the antibody is an anti-C1q antibody.

21-32. (canceled)

33. The method of claim 20, wherein the antibody is a monoclonal antibody, a polyclonal antibody, a recombinant antibody, a humanized antibody, a chimeric antibody, a multispecific antibody, antibody fragments, or an antibody derivative thereof.

34. The method of claim 33, wherein the antibody fragment is a Fab fragment, a Fab′ fragment, a F(ab′)2 fragment, a Fv fragment, a diabody, or a single chain antibody molecule.

35-36. (canceled)

37. The method of claim 20, wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7.

38. The method of claim 20, wherein the antibody comprises a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11.

39. The method of claim 20, wherein the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7.

40. The method of claim 39, wherein the light chain variable domain comprising an amino acid sequence selected from SEQ ID NO: 4 and 35-38.

41. The method of claim 20, wherein the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11.

42. The method of claim 41, wherein the heavy chain variable domain comprising an amino acid sequence selected from SEQ ID NO: 8 and 31-34.

43. The method of claim 33, wherein the antibody fragment comprises heavy chain Fab fragment of SEQ ID NO: 39 and light chain Fab fragment of SEQ ID NO: 40.

44. The method of claim 1, wherein the inhibitor of the classical complement pathway is a C1r inhibitor.

45-52. (canceled)

53. The method of claim 1, wherein the inhibitor of the classical complement pathway is a C1s inhibitor.

54-61. (canceled)

62. The method of claim 1, wherein the inhibitor of the classical complement pathway is an anti-C1 complex antibody, optionally wherein the anti-C1 complex antibody inhibits C1r or C1s activation or prevents their ability to act on C2 or C4.

63-85. (canceled)

86. A method of determining a subject's risk of developing epilepsy due to a traumatic brain injury, hypoxic brain injury, brain infection, stroke, or genetic syndrome, comprising:
(a) administering an anti-C1q, anti-C1r, or anti-C1s antibody to the subject, wherein the anti-C1q, anti-C1r, or anti-C1s antibody is coupled to a detectable label;
(b) detecting the detectable label to measure the amount or location of C1q, C1r, or C1s in the subject; and
(c) comparing the amount or location of one or more of C1q, C1r, or C1s to a reference, wherein the risk of developing epilepsy is characterized based on the comparison of the amount or location of one or more of C1q, C1r, or C1s to the reference.

87-98. (canceled)


SEQUENCE LISTING (86 sequences)

SEQ ID NO: 1 - protein, 245 aa, Homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 253 aa, Homo sapiens
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 3 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 4 - protein, 107 aa, Artificial Sequence
  Description of Artificial Sequence: Synthetic polypeptide
DVQITQSPSYLAASPGETITINCRASKSINKYLAWYQEKPGKTNKLLIYSGSTLQSGIPSRFSGSGSGTDFTLTISSLEPEDFAMYYCQQHNEYPLTFGAGTKLELK

SEQ ID NO: 5 - protein, 11 aa, Artificial Sequence
  Description of Artificial Sequence: Synthetic peptide
RASKSINKYLA

SEQ ID NO: 6 - protein, 7 aa, Artificial Sequence
  Description of Artificial Sequence: Synthetic peptide
SGSTLQS

SEQ ID NO: 7 - protein, 9 aa, Artificial Sequence
  Description of Artificial Sequence: Synthetic peptide
QQHNEYPLT

SEQ ID NO: 8 - protein, 121 aa, Artificial Sequence
  Description of Artificial Sequence: Synthetic polypeptide
QVQLQQPGAELVKPGASVKLSCKSSGYHFTSYWMHWVKQRPGQGLEWIGVIHPNSGSINYNEKFESKATLTVDKSSSTAYMQLSSLTSEDSAVYYCAGERDSTEVLPMDYWGQGTSVTVSS

SEQ ID NO: 9 - protein, 10 aa, Artificial Sequence
  Description of Artificial Sequence: Synthetic peptide
GYHFTSYWMH

SEQ ID NO: 10 - protein, 17 aa, Artificial Sequence
  Description of Artificial Sequence: Synthetic peptide
VIHPNSGSINYNEKFES

SEQ ID NO: 11 - protein, 12 aa, Artificial Sequence
  Description of Artificial Sequence: Synthetic peptide
ERDSTEVLPMDY

SEQ ID NO: 12 - DNA, 321 bp, Artificial Sequence
  Description of Artificial Sequence: Synthetic polynucleotide
gatgtccagataacccagtctccatcttatcttgctgcatctcctggagaaaccattactattaattgcagggcaagtaagagcattaacaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacaacataatgaatacccgctcacgttcggtgctgggaccaagctggagctgaaa

SEQ ID NO: 13 - DNA, 363 bp, Artificial Sequence
  Description of Artificial Sequence: Synthetic polynucleotide
caggtccaactgcagcagcctggggctgagctggtaaagcctggggcttcagtgaagttgtcctgcaagtcttctggctaccatttcaccagctactggatgcactgggtgaagcagaggcctggacaaggccttgagtggattggagtgattcatcctaatagtggtagtattaactacaatgagaagttcgagagcaaggccacactgactgtagacaaatcctccagcacagcctacatgcaactcagcagcctgacatctgaggactcggcggtctattattgtgcaggagagagagattctacggaggttctccctatggactactggggtcaaggaacctcagtcaccgtctcctca

SEQ ID NO: 14-15 - skipped (2 entries)

SEQ ID NO: 16 - protein, 31 aa, Homo sapiens
GLFQVVSGGMVLQLQQGDQVWVEKDPKKGHI

SEQ ID NO: 17 - protein, 26 aa, Homo sapiens
GLFQVVSGGMVLQLQQGDQVWVEKDP

SEQ ID NO: 18 - protein, 20 aa, Homo sapiens
SGGMVLQLQQGDQVWVEKDP

SEQ ID NO: 19 - protein, 18 aa, Homo sapiens
SGGMVLQLQQGDQVWVEK

SEQ ID NO: 20 - protein, 23 aa, Homo sapiens
WLAVNDYYDMVGIQGSDSVFSGF

SEQ ID NO: 21 - protein, 16 aa, Homo sapiens
YDMVGIQGSDSVFSGF

SEQ ID NO: 22 - protein, 8 aa, Homo sapiens
YDMVGIQG

SEQ ID NO: 23 - protein, 23 aa, Homo sapiens
HTANLCVLLYRSGVKVVTFCGHT

SEQ ID NO: 24 - protein, 9 aa, Homo sapiens
RSGVKVVTF

SEQ ID NO: 25-30 - skipped (6 entries)

SEQ ID NO: 31 - protein, 121 aa, Artificial Sequence [sequence omitted]

SEQ ID NO: 32 - protein, 121 aa, Artificial Sequence [sequence omitted]

SEQ ID NO: 33 - protein, 121 aa, Artificial Sequence
  Description of Artificial Sequence: Synthetic polypeptide
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSS

SEQ ID NO: 34 - protein, 121 aa, Artificial Sequence [sequence omitted]

SEQ ID NO: 35 - protein, 107 aa, Artificial Sequence [sequence omitted]

SEQ ID NO: 36 - protein, 107 aa, Artificial Sequence [sequence omitted]

SEQ ID NO: 37 - protein, 107 aa, Artificial Sequence
  Description of Artificial Sequence: Synthetic polypeptide
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIK

SEQ ID NO: 38 - protein, 107 aa, Artificial Sequence [sequence omitted]

SEQ ID NO: 39 - protein, 229 aa, Artificial Sequence
  Description of Artificial Sequence: Synthetic polypeptide
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 40 - protein, 214 aa, Artificial Sequence
  Description of Artificial Sequence: Synthetic polypeptide
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41-46 - skipped (6 entries)

SEQ ID NO: 47 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48-50 - skipped (3 entries)

SEQ ID NO: 51 - protein, 12 aa, Artificial Sequence
  Description of Artificial Sequence: Synthetic peptide
SSVSSSYLHWYQ

SEQ ID NO: 52 - protein, 10 aa, Artificial Sequence
  Description of Artificial Sequence: Synthetic peptide
STSNLASGVP

SEQ ID NO: 53 - protein, 10 aa, Artificial Sequence
  Description of Artificial Sequence: Synthetic peptide
HQYYRLPPIT

SEQ ID NO: 54 - protein, 12 aa, Artificial Sequence
  Description of Artificial Sequence: Synthetic peptide
GFTFSNYAMSWV

SEQ ID NO: 55 - protein, 10 aa, Artificial Sequence
  Description of Artificial Sequence: Synthetic peptide
ISSGGSHTYY

SEQ ID NO: 56 - protein, 11 aa, Artificial Sequence
  Description of Artificial Sequence: Synthetic peptide
ARLFTGYAMDY

SEQ ID NO: 57 - protein, 109 aa, Artificial Sequence
  Description of Artificial Sequence: Synthetic polypeptide
DIVMTQTTAIMSASLGERVTMTCTASSSVSSSYLHWYQQKPGSSPKLWIYSTSNLASGVPARFSGSGSGTFYSLTISSMEAEDDATYYCHQYYRLPPITFGAGTKLELK

SEQ ID NO: 58 - protein, 115 aa, Artificial Sequence
  Description of Artificial Sequence: Synthetic polypeptide
QVKLEESGGALVKPGGSLKLSCAASGFTFSNYAMSWVRQIPEKRLEWVATISSGGSHTYYLDSVKGRFTISRDNARDTLYLQMSSLRSEDTALYYCARLFTGYAMDYWGQGTSVT

SEQ ID NO: 59-61 - skipped (3 entries)

SEQ ID NO: 62 - protein, 12 aa, Artificial Sequence
  Description of Artificial Sequence: Synthetic peptide
TASSSVSSSYLH

SEQ ID NO: 63 - protein, 7 aa, Artificial Sequence
  Description of Artificial Sequence: Synthetic peptide
STSNLAS

SEQ ID NO: 64 - protein, 5 aa, Artificial Sequence
  Description of Artificial Sequence: Synthetic peptide
NYAMS

SEQ ID NO: 65 - protein, 17 aa, Artificial Sequence
  Description of Artificial Sequence: Synthetic peptide
TISSGGSHTYYLDSVKG

SEQ ID NO: 66 - protein, 9 aa, Artificial Sequence
  Description of Artificial Sequence: Synthetic peptide
LFTGYAMDY

SEQ ID NO: 67 - protein, 109 aa, Artificial Sequence
  Description of Artificial Sequence: Synthetic polypeptide
QIVLTQSPAIMSASLGERVTMTCTASSSVSSSYLHWYQQKPGSSPKLWIYSTSNLASGVPARFSGSGSGTFYSLTISSMEAEDDATYYCHQYYRLPPITFGAGTKLELK

SEQ ID NO: 68 - protein, 118 aa, Artificial Sequence
  Description of Artificial Sequence: Synthetic polypeptide
EVMLVESGGALVKPGGSLKLSCAASGFTFSNYAMSWVRQIPEKRLEWVATISSGGSHTYYLDSVKGRFTISRDNARDTLYLQMSSLRSEDTALYYCARLFTGYAMDYWGQGTSVTVSS

SEQ ID NO: 69 - DNA, 20 bp, Artificial Sequence
  Description of Artificial Sequence: Synthetic primer
atggagacctctcagggatg

SEQ ID NO: 70 - DNA, 20 bp, Artificial Sequence
  Description of Artificial Sequence: Synthetic primer
ataccagtccggatgccagc

SEQ ID NO: 71 - DNA, 20 bp, Artificial Sequence
  Description of Artificial Sequence: Synthetic primer
ataccagtccggatgccagc

SEQ ID NO: 72 - DNA, 25 bp, Artificial Sequence
  Description of Artificial Sequence: Synthetic primer
tcacccacactgtgcccatctacga

SEQ ID NO: 73 - DNA, 20 bp, Artificial Sequence
  Description of Artificial Sequence: Synthetic primer
gacaaggcaccttcagaacc

SEQ ID NO: 74 - DNA, 22 bp, Artificial Sequence
  Description of Artificial Sequence: Synthetic primer
cagcagcttagtcagggttaca

SEQ ID NO: 75 - DNA, 23 bp, Artificial Sequence
  Description of Artificial Sequence: Synthetic primer
gccatgcccaggtgcaagatcaa

SEQ ID NO: 76 - DNA, 20 bp, Artificial Sequence
  Description of Artificial Sequence: Synthetic primer
tggctggctgccctctgatg

SEQ ID NO: 77 - DNA, 22 bp, Artificial Sequence
  Description of Artificial Sequence: Synthetic primer
tggacagtggagcaactccggt

SEQ ID NO: 78 - DNA, 23 bp, Artificial Sequence
  Description of Artificial Sequence: Synthetic primer
ggtgggtactccacaggctggaa

SEQ ID NO: 79 - DNA, 20 bp, Artificial Sequence
  Description of Artificial Sequence: Synthetic primer
ctcatccgcgtttactccat

SEQ ID NO: 80 - DNA, 20 bp, Artificial Sequence
  Description of Artificial Sequence: Synthetic primer
tgttctgttcgatgctcagg

SEQ ID NO: 81 - DNA, 20 bp, Artificial Sequence
  Description of Artificial Sequence: Synthetic primer
agcaggtcatcaagtcaggc

SEQ ID NO: 82 - DNA, 20 bp, Artificial Sequence
  Description of Artificial Sequence: Synthetic primer
gatgtagctggtgttgggct

SEQ ID NO: 83 - DNA, 18 bp, Artificial Sequence
  Description of Artificial Sequence: Synthetic primer
accccctaaataacctgg

SEQ ID NO: 84 - DNA, 20 bp, Artificial Sequence
  Description of Artificial Sequence: Synthetic primer
cctcatgtatcctttttgga

SEQ ID NO: 85 - DNA, 20 bp, Artificial Sequence
  Description of Artificial Sequence: Synthetic primer
agggtactttgcctgctgaa

SEQ ID NO: 86 - DNA, 20 bp, Artificial Sequence
  Description of Artificial Sequence: Synthetic primer
tgtgaaggtgctcttggatg